(19) United States
(12) Patent Application Publication
(10) Pub. No.: US 2010/0025661 A1
(43) Pub. Date: Feb. 4, 2010

(54) Luminescent material and organic electroluminescent device using the same

(76) Inventors: Guofang Wang, Chiba (JP); Manabu Uchida, Chiba (JP); Youhei Ono, Chiba (JP)

Correspondence Address: WENDEROTH, LIND & PONACK, L.L.P., 1030 15th Street, N.W.,, Suite 400 East, Washington, DC, 20005-1503, US

(21) Appl. No.: 11/631,421

(22) PCT Filed: Jun. 24, 2005
(86) PCT No.: PCT/JP05/11599

§ 371 Date: Jan. 3, 2007

(30) Foreign Application Priority Data
Jul. 2, 2004 (JP) 2004-196986

(51) Int. Cl. H01L 51/54; C07D 333/50; C07C 15/30; C07C 15/28; C07D 209/86; C07D 333/10; C07D 401/04; C07D 215/04; C07D 211/80

(52) U.S. Cl. 257/40; 549/49; 585/26; 548/446; 549/80; 546/257; 546/173; 546/285; 257/E51.026; 257/E51.022

(57) ABSTRACT

The subject of the present invention is to provide an emission material which contributes to high emission efficiency, low drive voltage, excellent heat resistance and long life in an organic electroluminescent device, particularly an emission material which is excellent in emission of blue color. Further, the subject is to provide an organic electroluminescent device using the above emission material. The above subjects can be achieved by an emission material represented by Formula (1) and an organic electroluminescent device comprising the same.

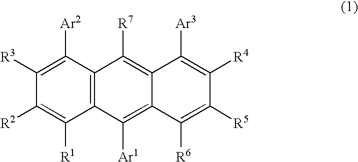

wherein R¹ to R⁷ are independently hydrogen, alkyl or cycloalkyl; Ar¹ is one selected from the group consisting of non-condensed aryl having 6 to 50 carbon atoms, 2-naphthyl, 9-phenanthryl, 6-chrysenyl, 2-triphenylenyl, 2-fluorenyl, 9-carbazolyl, 2-thienyl and 2-benzothienyl; and Ar² and Ar³ are independently non-condensed aryl having 6 to 50 carbon atoms, condensed aryl having 10 to 50 carbon atoms or heteroaryl having 2 to 50 carbon atoms.

### BACKGROUND OF THE INVENTION

The present invention relates to a novel emission material having an anthracene skeleton and an organic electroluminescent device (hereinafter abbreviated as an organic EL device) using the above emission material.

### RELATED ART

In recent years, attentions are paid to an organic EL device as a full color fiat panel in the subsequent generation, and emission materials of blue, green and red colors are actively researched and developed. Among the emission materials, particularly a blue color emission material is requested to be improved. Blue color emission materials which have so far been reported are distyrylarylene derivatives (refer to, for example, a patent document 1), zinc metal complexes (refer to, for example, a patent document 2), aluminum complexes (refer to, for example, a patent document 3), aromatic amine derivatives (refer to, for example, a patent document 4) and anthracene derivatives (refer to, for example, a patent document 5). Examples in which the anthracene derivatives are used for emission materials are disclosed in a non-patent document 1, a patent document 6, a patent document 7 and a patent document 8 in addition to the patent document 5. In the non-patent document 1, a 9,10-diphenylanthracene compound is used, but there used to be the problems that the crystallinity is high and that the ability to form a thin film is inferior. Organic EL devices using derivatives having an anthracene structure substituted with phenyls in 9 and 10 positions are disclosed as emission materials in the patent document 6, the patent document 7 and the patent document 8. Organic EL devices using anthracene derivatives substituted with naphthalenes in 9 and 10 positions are disclosed as emission materials in the patent document 5. However, any of the above compounds has symmetric molecular structure, and possibility of having high crystallinity is concerned. Organic EL devices using compounds having two or more anthracene rings as emission materials in order to reduce crystallinity to form a film having good amorphous state are proposed in a patent document 9, a patent document 10, a patent document 11 and a patent document 12. It is reported that emission of bluish green color is achieved by the above materials.

Patent document 1: JP H2-247278 A/1990
Patent document 2: JP H6-336586 A/1994
Patent document 3: JP H5-198378 A/1993
Patent document 4: JP H6-240248 A/1994
Patent document 5: JP H11-3782 A/1999
Patent document 6: JP H11-312588 A/1999
Patent document 7: JP H11-323323 A/1999
Patent document 8: JP H11-329732 A/1999
Patent document 9: JP H8-12600 A/1996
Patent document 10: JP H11-111458 A/1999
Patent document 11: JP H12-344691 A/2000
Patent document 12: JP H14-154993 A/2002
Non-patent document 1: Applied Physics Letters, 56 (9), 799 (1990)

### DISCLOSURE OF THE INVENTION

### Problems to be Solved by the Invention

The present invention has been made in light of the problems involved in such conventional techniques as described above, and an object of the present invention is to provide an emission material contributing to high emission efficiency, low drive voltage, excellent heat resistance and long life in an organic EL device, particularly an emission material which is excellent in emission of blue color. Further, an object of the present invention is to provide an organic EL device using the above emission material.

### Means for Solving the Problems

Intensive investigations repeated by the present inventors have resulted in finding that an organic EL device which has high emission efficiency, high luminance and long life and which can be driven at low voltage can be obtained by using alone for an emission layer of the organic EL device, a novel emission material having specific structure in which anthracene is fundamental structure and in which 1-position, 8-position and 10-position are independently replaced by aryl or heteroaryl or using it in combination with other emission materials, and they have completed the present invention based on the above knowledge.

Terms used in the present invention are defined as follows. Alkyl may be a linear group or a branched group. This applies to a case where optional —CH₂— in this group is replaced by —O— or arylene. The term “optional” used in the present invention shows that the position and the number are optional, and it means “at least one selected without distinguishing”. When plural groups or atoms are replaced by other groups, they each may be replaced by different groups. For example, a case where optional —CH₂— in alkyl may be replaced by —O— or phenylene shows that it may be any of alkoxyphenyl, alkoxyphenylalkyl, alkoxyalkylphenylalkyl, phenoxy, phenylalkoxy, phenylalkoxyalkyl, alkylphenoxy, alkylphenylalkoxy and alkylphenylalkoxyalkyl. The groups of alkoxy and alkoxyalkyl in the above groups may be linear groups or branched groups. Provided that when it is described in the present invention that optional —CH₂— may be replaced by —O—, a case where continuous plural —CH₂— are replaced by —O— is not included. Further, “an emission material represented by Formula (1)” is shown by “an emission material (1)” in the present specification.

The problems described above are solved by the respective items shown below.

[1] An emission material represented by the following Formula (1):

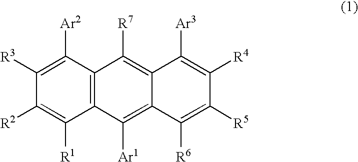

wherein R¹ to R⁷ are independently hydrogen, alkyl having 1 to 24 carbon atoms or cycloalkyl having 3 to 24 carbon atoms; optional —CH₂— in the above alkyl having 1 to 24 carbon atoms may be replaced by —O—, and optional —CH₂— other than —CH₂— directly bonded to the anthracene ring may be replaced by arylene having 6 to 24 carbon atoms; optional hydrogens in the above cycloalkyl having 3 to 24 carbon atoms may be replaced by alkyl having 1 to 24 carbon atoms or aryl having 6 to 50 carbon atoms;
Ar¹ is one selected from the group consisting of non-condensed aryl having 6 to 50 carbon atoms, 2-naphthyl, 9-phenanthryl, 6-chrysenyl, 2-triphenylenyl, 2-fluorenyl, 9-carbazolyl, 2-thienyl and 2-benzothienyl;
optional hydrogens in the above groups may be replaced by alkyl having 1 to 24 carbon atoms, cycloalkyl having 3 to 12 carbon atoms, aryl having 6 to 24 carbon atoms or heteroaryl; optional —CH₂— in the above alkyl having 1 to 24 carbon atoms may be replaced by —O—, and optional —CH₂— other than —CH₂— directly bonded to the above groups may be replaced by arylene having 6 to 24 carbon atoms; optional hydrogens in the above cycloalkyl having 3 to 24 carbon atoms may be replaced by alkyl having 1 to 24 carbon atoms or aryl having 6 to 24 carbon atoms; optional hydrogens in the above aryl having 6 to 24 carbon atoms may be replaced by alkyl having 1 to 12 carbon atoms, cycloalkyl having 3 to 12 carbon atoms or aryl having 6 to 24 carbon atoms, and optional hydrogens in the above heteroaryl may be replaced by alkyl having 1 to 12 carbon atoms, cycloalkyl having 3 to 12 carbon atoms or aryl having 6 to 24 carbon atoms; and
Ar² and Ar³ are independently non-condensed aryl having 6 to 50 carbon atoms, condensed aryl having 10 to 50 carbon atoms or heteroaryl.
[2] The emission material as described in the above item 1, wherein R¹ to R⁷ are independently hydrogen, methyl or tert-butyl, and Ar¹ is non-condensed aryl having 6 to 50 carbon atoms.
[3] The emission material as described in the above item 1, wherein R¹ to R⁷ are independently hydrogen, methyl or tert-butyl, and Ar¹ is phenyl, biphenylyl, terphenylyl or quaterphenylyl.
[4] The emission material as described in the above item 1, wherein R¹ to R⁷ are independently hydrogen, methyl or tert-butyl, and Ar¹ is 2-naphthyl, 9-phenanthryl, 6-chrysenyl, 2-triphenylenyl, 2-fluorenyl, 9-carbazolyl, 2-thienyl or 2-benzothienyl.
[5] An emission material represented by the following Formula (1):

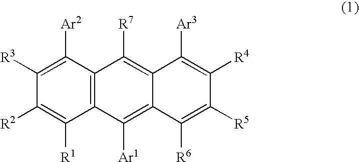

wherein R¹ to R⁷ are independently hydrogen, methyl or tert-butyl, and Ar¹ is non-condensed aryl represented by Formula (2);
Ar² and Ar³ are independently phenyl, 4-tert-butylphenyl, 4-(9-carbazolyl)phenyl, 2-biphenylyl, 3-biphenylyl, 4-biphenylyl, m-terphenyl-5′-yl, 3,5-di(2-naphthyl)phenyl, p-quaterphenyl-3′-yl, m-quaterphenyl-3-yl, o-quaterphenyl-2-yl, 1-naphthyl, 4-phenyl-1-naphthyl, 4-(9-carbazolyl)-1-naphthyl, 2-naphthyl, 6-(m-terphenyl-5′-yl)-2-naphthyl, 6-(2-naphthyl)-2-naphthyl, 9-phenanthryl, 2-benzothienyl or 3-phenyl-2-benzothienyl;

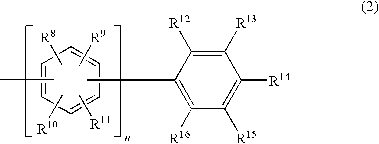

wherein n is an integer of 0 to 8;
R⁸ to R¹⁶ are independently hydrogen, alkyl having 1 to 24 carbon atoms, cycloalkyl having 3 to 24 carbon atoms, aryl having 6 to 24 carbon atoms or heteroaryl; optional —CH₂— in the above alkyl having 1 to 24 carbon atoms may be replaced by —O—, and optional —CH₂— other than —CH₂— directly bonded to the benzene ring may be replaced by arylene having 6 to 24 carbon atoms; optional hydrogens in the above cycloalkyl having 3 to 24 carbon atoms may be replaced by alkyl having 1 to 24 carbon atoms or aryl having 6 to 24 carbon atoms; optional hydrogens in the above aryl having 6 to 24 carbon atoms may be replaced by alkyl having 1 to 24 carbon atoms, cycloalkyl having 3 to 24 carbon atoms or aryl having 6 to 24 carbon atoms; and optional hydrogens in the above heteroaryl may be replaced by alkyl having 1 to 12 carbon atoms, cycloalkyl having 3 to 12 carbon atoms or aryl having 6 to 24 carbon atoms.
[6] The emission material as described in the above item 5, wherein Ar¹ is phenyl, biphenylyl, terphenylyl or quaterphenylyl in which optional hydrogens may be replaced by methyl, tert-butyl, phenyl, 2-naphthyl, 1-naphthyl, 2-benzothienyl, 3-phenyl-2-benzothienyl or 9-carbazolyl.
[7] The emission material as described in the above item 5, wherein Ar¹ is phenyl, 2-biphenylyl, 3-biphenylyl, 4-biphenylyl, m-terphenyl-5′-yl, m-quaterphenyl-3-yl or o-quaterphenyl-3-yl in which optional hydrogens may be replaced by methyl, tert-butyl, phenyl, 2-naphthyl, 1-naphthyl, 2-benzothienyl, 3-phenyl-2-benzothienyl or 9-carbazolyl.
[8] An emission material represented by the following Formula (1):

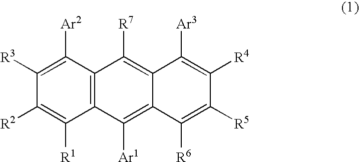

wherein R¹ to R⁷ are independently hydrogen, methyl or tert-butyl;
Ar¹ is 2-naphthyl, 9-phenanthryl, 6-chrysenyl, 2-triphenylenyl, 2-fluorenyl, 9-carbazolyl, 2-thienyl or 2-benzothienyl in which optional hydrogens may be replaced by methyl, tert-butyl, phenyl, m-terphenyl-5′-yl, 2-naphthyl, 1-naphthyl, 2-benzothienyl, 3-phenyl-2-benzothienyl or 9-carbazolyl; and
Ar² and Ar³ are independently phenyl, 4-tert-butylphenyl, 4-(9-carbazolyl)phenyl, 2-biphenylyl, 3-biphenylyl, 4-biphenylyl, m-terphenyl-5′-yl, 3,5-di(2-naphthyl)phenyl, p-quaterphenyl-3′-yl, m-quaterphenyl-3-yl, o-quaterphenyl-2-yl, 1-naphthyl, 4-phenyl-1-naphthyl, 4-(9-carbazolyl)-1-naphthyl, 2-naphthyl, 6-(m-terphenyl-5′-yl)-2-naphthyl, 6-(2-naphthyl)-2-naphthyl, 9-phenanthryl, 2-benzothienyl or 3-phenyl-2-benzothienyl.
[9] The emission material as described in any of the above items 5 to 7, wherein Ar¹ is one selected from phenyl, 4-tert-butylphenyl and 4-(9-carbazolyl)phenyl.
[10] The emission material as described in any of the above items 5 to 7, wherein Ar¹ is one selected from 2-biphenylyl, 3-biphenylyl and 4-biphenylyl.
[11] The emission material as described in any of claims 5 to 7, wherein Ar¹ is m-terphenyl-5′-yl.
[12] The emission material as described in any of the above items 5 to 7, wherein Ar¹ is 3,5-di(2-naphthyl)phenyl.
[13] The emission material as described in any of the above items 5 to 7, wherein Ar¹ is m-quaterphenyl-3-yl or o-quaterphenyl-2-yl.
[14] The emission material as described in the above item 8,

wherein Ar¹ is one selected from 2-naphthyl, 6-(m-terphenyl-5′-yl)-2-naphthyl, 6-(2-naphthyl)-2-naphthyl and 6-(9-carbazolyl)-2-naphthyl.

[15] The emission material as described in the above item 8, wherein Ar¹ is 9-phenanthryl.
[16] The emission material as described in the above item 8, wherein Ar¹ is 9-carbazolyl.
[17] The emission material as described in the above item 8, wherein Ar¹ is 2-benzothienyl or 3-phenyl-2-benzothienyl.
[18] The emission material as described in any of the above items 9 to 17, wherein R¹ to R⁶ are hydrogens; R⁷ is hydrogen or methyl; and Ar² and Ar³ are one selected from phenyl, 4-tert-butylphenyl and 4-(9-carbazolyl)phenyl.
[19] The emission material as described in any of the above items 9 to 17, wherein R¹ to R⁶ are hydrogens; R⁷ is hydrogen or methyl; and Ar² and Ar³ are one selected from 2-biphenylyl, 3-biphenylyl and 4-biphenylyl.
[20] The emission material as described in any of the above items 9 to 17, wherein R¹ to R⁶ are hydrogens; R⁷ is hydrogen or methyl; and Ar² and Ar³ are m-terphenyl-5′-yl.
[21] The emission material as described in any of the above items 9 to 17, wherein R¹ to R⁶ are hydrogens; R⁷ is hydrogen or methyl; and Ar² and Ar³ are 3,5-di(2-naphthyl)phenyl.
[22] The emission material as described in any of the above items 9 to 17, wherein R¹ to R⁶ are hydrogens; R⁷ is hydrogen or methyl; and Ar² and Ar³ are one selected from p-quaterphenyl-3′-yl, m-quaterphenyl-3-yl and o-quaterphenyl-2-yl.
[23] The emission material as described in any of the above items 9 to 17, wherein R¹ to R⁶ are hydrogens; R⁷ is hydrogen or methyl; and Ar² and Ar³ are one selected from 1-naphthyl, 4-phenyl-1-naphthyl and 4-(9-carbazolyl)-1-naphthyl.
[24] The emission material as described in any of the above items 9 to 17, wherein R¹ to R⁶ are hydrogens; R⁷ is hydrogen or methyl; and Ar² and Ar³ are one selected from 2-naphthyl, 6-(m-terphenyl-5′-yl)-2-naphthyl and 6-(2-naphthyl)-2-naphthyl.
[25] The emission material as described in any of the above items 9 to 17, wherein R¹ to R⁶ are hydrogens; R⁷ is hydrogen or methyl; and Ar² and Ar³ are 9-phenanthryl.
[26] The emission material as described in any of the above items 9 to 18, wherein R¹ to R⁶ are hydrogens; R⁷ is hydrogen or methyl; and Ar² and Ar³ are 2-benzothienyl or 3-phenyl-2-benzothienyl.
[27] An organic electroluminescent device comprising a substrate and provided thereon at least a hole transport layer, an emission layer and an electron transport layer which are sandwiched between an anode and a cathode, wherein the above emission layer comprises the emission material as described in the above items 1 to 26.

### EFFECTS OF THE INVENTION

The emission material of the present invention can be used for emission of various colors, and it is particularly excellent in emission of blue color. Use of the above emission material makes it possible to provide an organic EL device having high emission efficiency, low drive voltage, excellent heat resistance and long life. Use of the organic EL device of the present invention makes it possible to produce a display unit having a high performance used for full color display.

### BEST MODE FOR CARRYING OUT THE INVENTION

The present invention shall be explained below in further details.

The first present invention is an emission material having an anthracene skeleton represented by Formula (1):

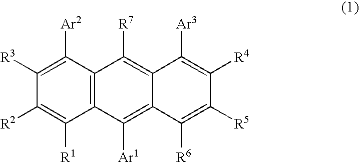

In Formula (1), R¹ to R⁷ are independently hydrogen, alkyl having 1 to 24 carbon atoms or cycloalkyl having 3 to 24 carbon atoms. R¹ to R⁷ may be the same or different.

The examples of the alkyl having 1 to 24 carbon atoms are methyl, ethyl, n-propyl, isopropyl, n-butyl, isobutyl, sec-butyl, tert-butyl, n-pentyl, isopentyl, tert-pentyl, neopentyl, n-hexyl, isohexyl, 1-methylpentyl, 2-methylpentyl, n-hexyl, isohexyl, 1-methylpentyl, 2-methylpentyl and 5-methylhexyl.

Optional —CH₂— in the above alkyl having 1 to 24 carbon atoms may be replaced by —O—, and optional —CH₂— other than —CH₂— directly bonded to the anthracene ring may be replaced by arylene having 6 to 24 carbon atoms. The examples of the arylene having a carbon number of 6 to 24 are 1,2-phenylene, 1,3-phenylene, 1,4-phenylene, naphthalene-2,6-diyl and naphthalene-1,4-diyl. The preferred example of the arylene having 6 to 24 carbon atoms is 1,4-phenylene.

The examples of the alkyl having 1 to 24 carbon atoms in which optional —CH₂— is replaced by —O— are methoxy, ethoxy, propyloxy, isopropyloxy, n-butyloxy, isobutyloxy, sec-butyloxy, tert-butyloxy, n-pentyloxy, isopentyloxy, tert-pentyloxy, neopentyloxy, n-hexyloxy, isohexyloxy, 1-methylpentyloxy, 2-methylpentyloxy and n-hexyloxy.

The examples of the alkyl having 1 to 24 carbon atoms in which optional —CH₂— is replaced by arylene having 6 to 24 carbon atoms are 2-phenylethyl, 2-(4-methylphenyl)ethyl, 1-methyl-1-phenylethyl, 1,1-dimethyl-2-phenylethyl and trityl.

The examples of the alkyl having 1 to 24 carbon atoms in which optional —CH₂— is replaced by —O— and in which optional —CH₂— other than —CH₂— directly bonded to anthracene is replaced by arylene having 6 to 24 carbon atoms are phenoxy, o-tolyloxy, m-tolyloxy, p-tolyloxy, 1-naphthoxy, 2-naphthoxy, 2,4-dimethylphenoxy, 2,6-dimethylphenoxy, 2,4,6-trimethylphenoxy, 4-tert-butylphenoxy, 2,4-di-tert-butylphenoxy, 2,4,6-tri-tert-butylphenoxy, 2-phenylethoxy and 2-(4-methylphenyl)ethoxy.

The examples of the cycloalkyl having 3 to 24 carbon atoms are cyclopropyl, cyclobutyl, cyclopentyl and cyclohexyl. Optional hydrogens in the above cycloalkyl having 3 to 24 carbon atoms may be replaced by alkyl having 1 to 24 carbon atoms or aryl having 6 to 50 carbon atoms.

The examples of the cycloalkyl having 3 to 24 carbon atoms in which optional hydrogens are replaced by alkyl having 1 to 24 carbon atoms are 2-methylcyclohexyl, 3-methylcyclohexyl, 4-methylcyclohexyl, 2,4,6-trimethylcyclohexyl, 2-tert-butylcyclohexyl, 3-tert-butylcyclohexyl, 4-tert-butylcyclohexyl and 2,4,6-tri-tert-butylcyclohexyl.

The examples of the cycloalkyl having 3 to 24 carbon atoms in which optional hydrogens are replaced by aryl having 6 to 50 carbon atoms are 2-phenylcyclohexyl, 3-phenylcyclohexyl, 4-phenylcyclohexyl, 2,4-diphenylcyclohexyl and 3,5-diphenylcyclohexyl.

The preferred examples of R¹ to R⁷ are hydrogen, methyl and tert-butyl, and the more preferred examples of R⁷ are hydrogen and methyl.

Ar¹ is one selected from the group consisting of non-condensed aryl having 6 to 50 carbon atoms, 2-naphthyl, 9-phenanthryl, 6-chrysenyl, 2-triphenylenyl, 2-fluorenyl, 9-carbazolyl, 2-thienyl and 2-benzothienyl.

The non-condensed aryl having 6 to 50 carbon atoms is represented by Formula (2):

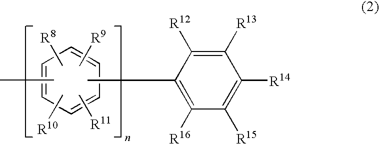

In Formula (2), n is an integer of 0 to 8, preferably 0 to 4. When n is an integer of 1 to 8, phenylene in the middle is independently optionally selected from 1,2-phenylene, 1,3-phenylene and 1,4-phenylene. If 1,2-phenylene is selected, an emission wavelength of a blue color originating in the fundamental skeleton can be maintained, and therefore it is preferred. If 1,4-phenylene is selected, the compound is characterized by that it is increased in rigidity, excellent in a heat resistance and extended in a life. 1,3-Phenylene brings characteristics positioned in the middle of both to the compound. Considering a wavelength, heat resistance and life which are expected to the emission material based on the design of the device, the conditions of the number of n and the kind of the phenylene are added, whereby the emission material meeting the objects can be obtained.

R⁸ to R¹⁶ are independently hydrogen, alkyl having 1 to 24 carbon atoms, cycloalkyl having 3 to 24 carbon atoms, aryl having 6 to 24 carbon atoms or heteroaryl.

The examples of the alkyl having 1 to 24 carbon atoms are methyl, ethyl, n-propyl, isopropyl, n-butyl, isobutyl, sec-butyl, tert-butyl, n-pentyl, isopentyl, tert-pentyl, neopentyl, n-hexyl, isohexyl, 1-methylpentyl, 2-methylpentyl, n-hexyl, isohexyl, 1-methylpentyl, 2-methylpentyl and 5-methylhexyl.

Optional —CH₂— in the above alkyl having 1 to 24 carbon atoms may be replaced by —O—, and optional —CH₂— other than —CH₂— directly bonded to the benzene ring may be replaced by arylene having 6 to 24 carbon atoms. The examples of the arylene having 6 to 24 carbon atoms are the same as described above, and the preferred example thereof is 1,4-phenylene.

The examples of the alkyl having 1 to 24 carbon atoms in which optional —CH₂— is replaced by —O— are methoxy, ethoxy, propyloxy, isopropyloxy, n-butyloxy, isobutyloxy, sec-butyloxy, tert-butyloxy, n-pentyloxy, isopentyloxy, tert-pentyloxy, neopentyloxy, n-hexyloxy, isohexyloxy, 1-methylpentyloxy, 2-methylpentyloxyloxy and n-hexyloxy.

The examples of the alkyl having 1 to 24 carbon atoms in which optional —CH₂— is replaced by arylene having 6 to 24 carbon atoms are 2-phenylethyl, 2-(4-methylphenyl)ethyl, 1-methyl-1-phenylethyl, 1,1-dimethyl-2-phenylethyl and trityl.

The examples of the alkyl having 1 to 24 carbon atoms in which optional —CH₂— is replaced by —O— and in which optional —CH₂— other than —CH₂— directly bonded to the benzene ring is replaced by arylene having 6 to 24 carbon atoms are phenoxy, o-tolyloxy, m-tolyloxy, p-tolyloxy, 1-naphthoxy, 2-naphthoxy, 2,4-dimethylphenoxy, 2,6-dimethylphenoxy, 2,4,6-trimethylphenoxy, 4-tert-butylphenoxy, 2,4-di-tert-butylphenoxy, 2,4,6-tri-tert-butylphenoxy, 2-phenylethoxy and 2-(4-methylphenyl)ethoxy.

The examples of the cycloalkyl having 3 to 24 carbon atoms are cyclopropyl, cyclobutyl, cyclopentyl and cyclohexyl.

Optional hydrogens in the above cycloalkyl having 3 to 24 carbon atoms may be replaced by alkyl having 1 to 24 carbon atoms or aryl having 6 to 24 carbon atoms.

The examples of the cycloalkyl having 3 to 24 carbon atoms in which optional hydrogens are replaced by alkyl having 1 to 24 carbon atoms are 2-methylcyclohexyl, 3-methylcyclohexyl, 4-methylcyclohexyl, 2,4,6-trimethylcyclohexyl, 2-tert-butylcyclohexyl, 3-tert-butylcyclohexyl, 4-tert-butylcyclohexyl and 2,4,6-tri-tert-butylcyclohexyl.

The examples of the cycloalkyl having 3 to 24 carbon atoms in which optional hydrogens are replaced by aryl having 6 to 24 carbon atoms are 2-phenylcyclohexyl, 3-phenylcyclohexyl, 4-phenylcyclohexyl, 2,4-diphenylcyclohexyl and 3,5-diphenylcyclohexyl.

The examples of the aryl having 6 to 24 carbon atoms are phenyl, 1-naphthyl, 2-naphthyl, 1-anthryl, 2-anthryl, 9-anthryl, 1-phenanthryl, 2-phenanthryl, 3-phenanthryl, 4-phenanthryl, 9-phenanthryl, 1-pyrenyl, 2-pyrenyl, 4-pyrenyl, 1-perylenyl, 2-perylenyl, 1-chrysenyl, 2-chrysenyl, 3-chrysenyl, 5-chrysenyl, 6-chrysenyl, 1-triphenylenyl, 2-triphenylenyl and 2-fluorenyl.

Optional hydrogens in the above aryl having 6 to 24 carbon atoms may be replaced by alkyl having 1 to 24 carbon atoms, cycloalkyl having 3 to 12 carbon atoms or aryl having 6 to 24 carbon atoms. The examples of the aryl having 6 to 24 carbon atoms in which optional hydrogens are replaced by the alkyl having 1 to 24 carbon atoms are o-tolyl, m-tolyl, p-tolyl, 2-biphenylyl, 3-biphenylyl, 4-biphenylyl, 2,4-dimethylphenyl, 2,6-dimethylphenyl, 2,4,6-trimethylphenyl, 4-tert-butylphenyl, 2,4-di-tert-butylphenyl, 2,4,6-tri-tert-butylphenyl, 4-methyl-1-naphthyl, 4-tert-butyl-1-naphthyl, 6-methyl-2-naphthyl, 6-tert-butyl-2-naphthyl, 4-methyl-1-anthryl, 4-tert-butyl-1-anthryl, 10-methyl-9-anthryl, 10-tert-butyl-9-anthryl and 9,9-dimethyl-2-fluorenyl.

The examples of the aryl having 6 to 24 carbon atoms in which optional hydrogens are replaced by the cycloalkyl having 3 to 12 carbon atoms are 2-cyclohexylphenyl, 3-cyclohexylphenyl, 4-cyclohexylphenyl, 2,4-dicyclohexylphenyl and 3,5-dicyclohexylphenyl.

The examples of the aryl having 6 to 24 carbon atoms in which optional hydrogens are replaced by the aryl having 6 to 24 carbon atoms are m-terphenyl-2′-yl, m-terphenyl-4′-yl, m-terphenyl-5′-yl, o-terphenyl-3′-yl, o-terphenyl-4′-yl, p-terphenyl-2′-yl, m-terphenyl-2-yl, m-terphenyl-3-yl, m-terphenyl-4-yl, o-terphenyl-2-yl, o-terphenyl-3-yl, o-terphenyl-4-yl, p-terphenyl-2-yl, p-terphenyl-3-yl, p-terphenyl-4-yl, 5′-phenyl-m-terphenyl-2-yl, 5′-phenyl-m-terphenyl-3-yl, 5′-phenyl-m-terphenyl-4-yl, m-quaterphenyl-2-yl, m-quaterphenyl-3-yl, m-quaterphenyl-4-yl, o-quaterphenyl-2-yl, o-quaterphenyl-3-yl, o-quaterphenyl-4-yl, 3,5-di(1-naphthyl)phenyl, 3,5-di(2-naphthyl)phenyl, 4-phenyl-1-naphthyl, 6-phenyl-2-naphthyl, 6-(2-naphthyl)-2-naphthyl, 6-(1-naphthyl)-2-naphthyl, 4-(2-naphthyl)-1-naphthyl, 4-(1-naphthyl)-1-naphthyl and 9,9-diphenyl-2-fluorenyl.

The examples of the heteroaryl are 1-pyrroryl, 2-pyrroryl, 3-pyrroryl, 2-pyridyl, 3-pyridyl, 4-pyridyl, 2,2′-bipyridyl-6-yl, 2,3′-bipyridyl-6-yl, 2,4′-bipyridyl-6-yl, 3,2′-bipyridyl-6-yl, 3,3′-bipyridyl-6-yl, 3,4′-bipyridyl-6-yl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 1,7-phenanthroline-2-yl, 1,7-phenanthroline-3-yl, 1,7-phenanthroline-4-yl, 1,7-phenanthroline-5-yl, 1,7-phenanthroline-6-yl, 1,7-phenanthroline-8-yl, 1,7-phenanthroline-9-yl, 1,7-phenanthroline-10-yl, 1,8-phenanthroline-2-yl, 1,8-phenanthroline-3-yl, 1,8-phenanthroline-4-yl, 1,8-phenanthroline-5-yl, 1,8-phenanthroline-6-yl, 1,8-phenanthroline-7-yl, 1,8-phenanthroline-9-yl, 1,8-phenanthroline-10-yl, 1,9-phenanthroline-2-yl, 1,9-phenanthroline-3-yl, 1,9-phenanthroline-4-yl, 1,9-phenanthroline-5-yl, 1,9-phenanthroline-6-yl, 1,9-phenanthroline-7-yl, 1,9-phenanthroline-8-yl, 1,9-phenanthroline-10-yl, 1,10-phenanthroline-2-yl, 1,10-phenanthroline-3-yl, 1,10-phenanthroline-4-yl, 1,10-phenanthroline-5-yl, 2,9-phenanthroline-1-yl, 2,9-phenanthroline-3-yl, 2,9-phenanthroline-4-yl, 2,9-phenanthroline-5-yl, 2,8-phenanthroline-1-yl, 2,8-phenanthroline-3-yl, 2,8-phenanthroline-4-yl, 2,8-phenanthroline-5-yl, 2,8-phenanthroline-6-yl, 2,8-phenanthroline-7-yl, 2,8-phenanthroline-9-yl, 2,8-phenanthroline-10-yl, 2,7-phenanthroline-1-yl, 2,7-phenanthroline-3-yl, 2,7-phenanthroline-4-yl, 2,7-phenanthroline-5-yl, 2,7-phenanthroline-6-yl, 2,7-phenanthroline-8-yl, 2,7-phenanthroline-9-yl, 2,7-phenanthroline-10-yl, 1-phenazinyl, 2-phenazinyl, 1-phenothiazinyl, 2-phenothiazinyl, 3-phenothiazinyl, 4-phenothiazinyl, 10-phenothiazinyl, 1-phenoxazinyl, 2-phenoxazinyl, 3-phenoxazinyl, 4-phenoxazinyl, 10-phenoxazinyl, 3-furazanyl, 2-thienyl, 3-thienyl, 2-benzothienyl, 3-benzothienyl, 4-benzothienyl, 5-benzothienyl, 6-benzothienyl, 7-benzothienyl, 1-isobenzothienyl, 3-isobenzothienyl, 4-isobenzothienyl, 5-isobenzothienyl, 6-isobenzothienyl and 7-isobenzothienyl.

Optional hydrogens in the above heteroaryl may be replaced by alkyl having 1 to 24 carbon atoms, cycloalkyl having 3 to 12 carbon atoms or aryl having 6 to 24 carbon atoms. The examples of the heteroaryl in which optional hydrogens are replaced by the alkyl having 1 to 24 carbon atoms are 5-methyl-2-thienyl, 5-methyl-3-thienyl, 2,5-dimethyl-3-thienyl, 3,4,5-trimethyl-2-thienyl, 3-methyl-2-benzothienyl, 2-methyl-3-benzothienyl, 2-methylpyrrole-1-yl, 2,5-dimethylpyrrole-1-yl, 2-methyl-1-indolyl, 2-tert-butyl-1-indolyl, 3-methyl-9-carbazolyl, 3,6-dimethyl-9-carbazolyl, 3,6-di-tert-butyl-9-carbazolyl and 9-methyl-3-carbazolyl.

The examples of the heteroaryl in which optional hydrogens are replaced by the cycloalkyl having 3 to 12 carbon atoms are 5-cyclohexyl-2-thienyl, 3-cyclohexyl-2-benzothienyl, 2-cyclohexyl-3-benzothienyl, 3-cyclohexyl-9-carbazolyl, 3,6-dicyclohexyl-9-carbazolyl and 9-cyclohexyl-3-carbazolyl.

The examples of the heteroaryl in which optional hydrogens are replaced by the aryl having 6 to 24 carbon atoms are 5-phenyl-2-thienyl, 5-(1-naphthyl)-2-thienyl, 5-(2-naphthyl)-2-thienyl, 5-phenyl-3-thienyl, 2,5-diphenyl-3-thienyl, 2-phenyl-5-(1-naphthyl)-3-thienyl, 2-phenyl-5-(2-naphthyl)-3-thienyl, 3,4,5-triphenyl-2-thienyl, 3,4-diphenyl-5-(1-naphthyl)-2-thienyl, 3,4-diphenyl-5-(2-naphthyl)-2-thienyl, 3-phenyl-2-benzothienyl, 3-(1-naphthyl)-2-benzothienyl, 3-(2-naphthyl)-2-benzothienyl, 2-phenyl-3-benzothienyl, 3-phenyl-9-carbazolyl, 3-(1-naphthyl)-9-carbazolyl, 3-(2-naphthyl)-9-carbazolyl, 3,6-diphenyl-9-carbazolyl, 3,6-di(1-naphthyl)-9-carbazolyl, 3,6-di(2-naphthyl)-9-carbazolyl, 3,6-di(4-tert-butylphenyl)-9-carbazolyl, 9-phenyl-3-carbazolyl, 9-(1-naphthyl)-3-carbazolyl and 9-(2-naphthyl)-3-carbazolyl.

In 2-naphthyl, 9-phenanthryl, 6-chrysenyl, 2-triphenylenyl, 2-fluorenyl, 9-carbazolyl, 2-thienyl and 2-benzothienyl, optional hydrogens in the above rings may be replaced by alkyl having 1 to 24 carbon atoms, cycloalkyl having 3 to 24 carbon atoms, aryl having 6 to 24 carbon atoms or heteroaryl.

If hydrogen in a position adjacent to an atom bonded to anthracene in 2-naphthyl, 9-phenanthryl, 6-chrysenyl, 2-triphenylenyl, 2-fluorenyl, 9-carbazolyl, 2-thienyl or 2-benzothienyl is substituted with a substituent, an emission wavelength of a blue color originating in a fundamental skeleton thereof can be maintained, and it is suited to blue color emission. If hydrogens in the other positions are substituted, the compound is increased in rigidity and excellent in heat resistance. The emission material meeting the object can be obtained by suitably selecting the number of the substituents and the positions thereof considering emission wavelength and heat resistance expected to the emission material based on the design of the device.

The examples of the alkyl having 1 to 24 carbon atoms are methyl, ethyl, n-propyl, isopropyl, n-butyl, isobutyl, sec-butyl, tert-butyl, n-pentyl, isopentyl, tert-pentyl, neopentyl, n-hexyl, isohexyl, 1-methylpentyl, 2-methylpentyl, n-hexyl, isohexyl, 1-methylpentyl, 2-methylpentyl and 5-methylhexyl.

Optional —CH₂— in the above alkyl having 1 to 24 carbon atoms may be replaced by —O—, and optional —CH₂— other than —CH₂— bonded directly to the groups described above may be replaced by arylene having 6 to 24 carbon atoms. The examples of the arylene having 6 to 24 carbon atoms are the same as described above, and the preferred example thereof is 1,4-phenylene.

The examples of the alkyl having 1 to 24 carbon atoms in which optional —CH₂— is replaced by —O— are methoxy, ethoxy, propyloxy, isopropyloxy, n-butyloxy, isobutyloxy, sec-butyloxy, tert-butyloxy, n-pentyloxy, isopentyloxy, tert-pentyloxy, neopentyloxy, n-hexyloxy, isohexyloxy, 1-methylpentyloxy, 2-methylpentyloxy and n-hexyloxy.

The examples of the alkyl having 1 to 24 carbon atoms in which optional —CH₂— is replaced by arylene having 6 to 24 carbon atoms are 2-phenylethyl, 2-(4-methylphenyl)ethyl, 1-methyl-1-phenylethyl, 1,1-dimethyl-2-phenylethyl and trityl.

The examples of the alkyl having 1 to 24 carbon atoms in which optional —CH₂— is replaced by —O— and in which optional —CH₂— other than —CH₂— bonded directly to the groups described above is replaced by arylene having 6 to 24 carbon atoms are phenoxy, o-tolyloxy, m-tolyloxy, p-tolyloxy, 1-naphthoxy, 2-naphthoxy, 2,4-dimethylphenoxy, 2,6-dimethylphenoxy, 2,4,6-trimethylphenoxy, 4-tert-butylphenoxy, 2,4-di-tert-butylphenoxy, 2,4,6-tri-tert-butylphenoxy, 2-phenylethoxy and 2-(4-methylphenyl)ethoxy.

The examples of the cycloalkyl having 3 to 24 carbon atoms are cyclopropyl, cyclobutyl, cyclopentyl and cyclohexyl. Optional hydrogens in the above cycloalkyl having 3 to 24 carbon atoms may be replaced by alkyl having 1 to 24 carbon atoms or aryl having 6 to 24 carbon atoms.

The examples of the cycloalkyl having 3 to 24 carbon atoms in which optional hydrogens are replaced by the alkyl having 1 to 24 carbon atoms are 2-methylcyclohexyl, 3-methylcyclohexyl, 4-methylcyclohexyl, 2,4,6-trimethylcyclohexyl, 2-tert-butylcyclohexyl, 3-tert-butylcyclohexyl, 4-tert-butylcyclohexyl and 2,4,6-tri-tert-butylcyclohexyl.

The examples of the cycloalkyl having 3 to 24 carbon atoms in which optional hydrogens are replaced by the aryl having 6 to 24 carbon atoms are 2-phenylcyclohexyl, 3-phenylcyclohexyl, 4-phenylcyclohexyl, 2,4-diphenylcyclohexyl and 3,5-diphenylcyclohexyl.

The examples of the aryl having 6 to 24 carbon atoms are phenyl, 1-naphthyl, 2-naphthyl, 1-anthryl, 2-anthryl, 9-anthryl, 1-phenanthryl, 2-phenanthryl, 3-phenanthryl, 4-phenanthryl, 9-phenanthryl, 1-pyrenyl, 2-pyrenyl, 4-pyrenyl, 1-perylenyl, 2-perylenyl, 1-chrysenyl, 2-chrysenyl, 3-chrysenyl, 5-chrysenyl, 6-chrysenyl, 1-triphenylenyl, 2-triphenylenyl and 2-fluorenyl. Optional hydrogens in the above aryl having 6 to 24 carbon atoms may be replaced by alkyl having 1 to 24 carbon atoms, cycloalkyl having 3 to 12 carbon atoms or aryl having 6 to 24 carbon atoms.

The examples of the aryl having 6 to 24 carbon atoms in which optional hydrogens are replaced by the alkyl having 1 to 24 carbon atoms are o-tolyl, m-tolyl, p-tolyl, 2-biphenylyl, 3-biphenylyl, 4-biphenylyl, 2,4-dimethylphenyl, 2,6-dimethylphenyl, 2,4,6-trimethylphenyl, 4-tert-butylphenyl, 2,4-di-tert-butylphenyl, 2,4,6-tri-tert-butylphenyl, 4-methyl-1-naphthyl, 4-tert-butyl-1-naphthyl, 6-methyl-2-naphthyl, 6-tert-butyl-2-naphthyl, 4-methyl-1-anthryl, 4-tert-butyl-1-anthryl, 10-methyl-9-anthryl, 10-tert-butyl-9-anthryl and 9,9-dimethyl-2-fluorenyl.

The examples of the aryl having 6 to 24 carbon atoms in which optional hydrogens are replaced by the cycloalkyl having 3 to 12 carbon atoms are 2-cyclohexylphenyl, 3-cyclohexylphenyl, 4-cyclohexylphenyl, 2,4-dicyclohexylphenyl and 3,5-dicyclohexylphenyl.

The examples of the aryl having 6 to 24 carbon atoms in which optional hydrogens are replaced by the aryl having 6 to 24 carbon atoms are m-terphenyl-2′-yl, m-terphenyl-4′-yl, m-terphenyl-5′-yl, o-terphenyl-3′-yl, o-terphenyl-4′-yl, p-terphenyl-2′-yl, m-terphenyl-2-yl, m-terphenyl-3-yl, m-terphenyl-4-yl, o-terphenyl-2-yl, o-terphenyl-3-yl, o-terphenyl-4-yl, p-terphenyl-2-yl, p-terphenyl-3-yl, p-terphenyl-4-yl, 51-phenyl-m-terphenyl-2-yl, 5′-phenyl-m-terphenyl-3-yl, 5′-phenyl-m-terphenyl-4-yl, m-quaterphenyl-2-yl, m-quaterphenyl-3-yl, m-quaterphenyl-4-yl, o-quaterphenyl-2-yl, o-quaterphenyl-3-yl, o-quaterphenyl-4-yl, 3,5-di(1-naphthyl)-phenyl, 3,5-di(2-naphthyl)-phenyl, 4-phenyl-1-naphthyl, 6-phenyl-2-naphthyl, 6-(2-naphthyl)-2-naphthyl, 6-(1-naphthyl)-2-naphthyl, 4-(2-naphthyl)-1-naphthyl, 4-(1-naphthyl)-1-naphthyl and 9,9-diphenyl-2-fluorenyl.

The examples of the heteroaryl are 1-pyrroryl, 2-pyrroryl, 3-pyrroryl, 2-pyridyl, 3-pyridyl, 4-pyridyl, 2,2′-bipyridyl-6-yl, 2,3′-bipyridyl-6-yl, 2,4′-bipyridyl-6-yl, 3,2′-bipyridyl-6-yl, 3,3′-bipyridyl-6-yl, 3,4′-bipyridyl-6-yl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 1,7-phenanthroline-2-yl, 1,7-phenanthroline-3-yl, 1,7-phenanthroline-4-yl, 1,7-phenanthroline-5-yl, 1,7-phenanthroline-6-yl, 1,7-phenanthroline-8-yl, 1,7-phenanthroline-9-yl, 1,7-phenanthroline-10-yl, 1,8-phenanthroline-2-yl, 1,8-phenanthroline-3-yl, 1,8-phenanthroline-4-yl, 1,8-phenanthroline-5-yl, 1,8-phenanthroline-6-yl, 1,8-phenanthroline-7-yl, 1,8-phenanthroline-9-yl, 1,8-phenanthroline-10-yl, 1,9-phenanthroline-2-yl, 1,9-phenanthroline-3-yl, 1,9-phenanthroline-4-yl, 1,9-phenanthroline-5-yl, 1,9-phenanthroline-6-yl, 1,9-phenanthroline-7-yl, 1,9-phenanthroline-8-yl, 1,9-phenanthroline-10-yl, 1,10-phenanthroline-2-yl, 1,10-phenanthroline-3-yl, 1,10-phenanthroline-4-yl, 1,10-phenanthroline-5-yl, 2,9-phenanthroline-1-yl, 2,9-phenanthroline-3-yl, 2,9-phenanthroline-4-yl, 2,9-phenanthroline-5-yl, 2,8-phenanthroline-1-yl, 2,8-phenanthroline-3-yl, 2,8-phenanthroline-4-yl, 2,8-phenanthroline-5-yl, 2,8-phenanthroline-6-yl, 2,8-phenanthroline-7-yl, 2,8-phenanthroline-9-yl, 2,8-phenanthroline-10-yl, 2,7-phenanthroline-1-yl, 2,7-phenanthroline-3-yl, 2,7-phenanthroline-4-yl, 2,7-phenanthroline-5-yl, 2,7-phenanthroline-6-yl, 2,7-phenanthroline-8-yl, 2,7-phenanthroline-9-yl, 2,7-phenanthroline-10-yl, 1-phenazinyl, 2-phenazinyl, 1-phenothiazinyl, 2-phenothiazinyl, 3-phenothiazinyl, 4-phenothiazinyl, 10-phenothiazinyl, 1-phenoxazinyl, 2-phenoxazinyl, 3-phenoxazinyl, 4-phenoxazinyl, 10-phenoxazinyl, 3-furazanyl, 2-thienyl, 3-thienyl, 2-benzothienyl, 3-benzothienyl, 4-benzothienyl, 5-benzothienyl, 6-benzothienyl, 7-benzothienyl, 1-isobenzothienyl, 3-isobenzothienyl, 4-isobenzothienyl, 5-isobenzothienyl, 6-isobenzothienyl and 7-isobenzothienyl.

Optional hydrogens in the above heteroaryl may be replaced by alkyl having 1 to 24 carbon atoms, cycloalkyl having 3 to 12 carbon atoms or aryl having 6 to 24 carbon atoms.

The examples of the heteroaryl in which optional hydrogens are replaced by with the alkyl having 1 to 24 carbon atoms are 5-methyl-2-thienyl, 5-methyl-3-thienyl, 2,5-dimethyl-3-thienyl, 3,4,5-trimethyl-2-thienyl, 3-methyl-2-benzothienyl, 2-methyl-3-benzothienyl, 2-methylpyrrole-1-yl, 2,5-dimethylpyrrole-1-yl, 2-methyl-1-indolyl, 2-tert-butyl-1-indolyl, 3-methyl-9-carbazolyl, 3,6-dimethyl-9-carbazolyl, 3,6-di-tert-butyl-9-carbazolyl and 9-methyl-3-carbazolyl.

The examples of the heteroaryl in which optional hydrogens are replaced by the cycloalkyl having 3 to 12 carbon atoms are 5-cyclohexyl-2-thienyl, 3-cyclohexyl-2-benzothienyl, 2-cyclohexyl-3-benzothienyl, 3-cyclohexyl-9-carbazolyl, 3,6-dicyclohexyl-9-carbazolyl and 9-cyclohexyl-3-carbazolyl.

The examples of the heteroaryl in which optional hydrogens are replaced by the aryl having 6 to 24 carbon atoms are 5-phenyl-2-thienyl, 5-(1-naphthyl)-2-thienyl, 5-(2-naphthyl)-2-thienyl, 5-phenyl-3-thienyl, 2,5-diphenyl-3-thienyl, 2-phenyl-5-(1-naphthyl)-3-thienyl, 2-phenyl-5-(2-naphthyl)-3-thienyl, 3,4,5-triphenyl-2-thienyl, 3,4-diphenyl-5-(1-naphthyl)-2-thienyl, 3,4-diphenyl-5-(2-naphthyl)-2-thienyl, 3-phenyl-2-benzothienyl, 3-(1-naphthyl)-2-benzothienyl, 3-(2-naphthyl)-2-benzothienyl, 2-phenyl-3-benzothienyl, 3-phenyl-9-carbazolyl, 3-(1-naphthyl)-9-carbazolyl, 3-(2-naphthyl)-9-carbazolyl, 3,6-diphenyl-9-carbazolyl, 3,6-di(1-naphthyl)-9-carbazolyl, 3,6-di(2-naphthyl)-9-carbazolyl, 3,6-di(4-tert-butylphenyl)-9-carbazolyl, 9-phenyl-3-carbazolyl, 9-(1-naphthyl)-3-carbazolyl and 9-(2-naphthyl)-3-carbazolyl.

The preferred examples of Ar¹ are phenyl, 2-biphenylyl, 3-biphenylyl, 4-biphenylyl, 2,6-dimethylphenyl, 2,4,6-trimethylphenyl, 4-tert-butylphenyl, 2,4-di-tert-butylphenyl, m-terphenyl-4′-yl, m-terphenyl-5′-yl, p-terphenyl-2′-yl, m-terphenyl-2-yl, m-terphenyl-3-yl, o-terphenyl-2-yl, o-terphenyl-3-yl, m-quaterphenyl-3-yl, o-quaterphenyl-2-yl, 3,5-di(2-naphthyl)phenyl, 3,5-di(1-naphthyl)phenyl, 4-(9-carbazolyl)phenyl, 3,5-di(9-carbazolyl)phenyl, 2-naphthyl, 6-phenyl-2-naphthyl, 6-(m-terphenyl-5′-yl)-2-naphthyl, 6-(2-naphthyl)-2-naphthyl, 6-(9-carbazolyl)-2-naphthyl, 9-phenanthryl, 6-chrysenyl, 2-triphenylenyl, 9,9-dimethyl-2-fluorenyl, 9,9-diphenyl-2-fluorenyl, 5-phenyl-2-thienyl, 2,5-diphenyl-3-thienyl, 3,4,5-triphenyl-2-thienyl, 2-benzothienyl, 3-phenyl-2-benzothienyl, 2-phenyl-3-benzothienyl, 9-carbazolyl and 3,6-diphenyl-9-carbazolyl.

The more preferred examples of Ar¹ are phenyl, 2-biphenylyl, 3-biphenylyl, 4-biphenylyl, 4-tert-butylphenyl, m-terphenyl-5′-yl, m-quaterphenyl-3-yl, o-quaterphenyl-2-yl, 3,5-di(2-naphthyl)phenyl, 4-(9-carbazolyl)phenyl, 2-naphthyl, 6-(2-naphthyl)-2-naphthyl, 6-(9-carbazolyl)-2-naphthyl, 9-phenanthryl, 2-benzothienyl, 3-phenyl-2-benzothienyl and 9-carbazolyl.

Ar² and Ar³ are independently non-condensed aryl having 6 to 50 carbon atoms, condensed aryl having 10 to 50 carbon atoms or heteroaryl. The non-condensed aryl having 6 to 50 carbon atoms is the same as the non-condensed aryl having 6 to 50 carbon atoms in Ar¹ described above. Ar² and Ar³ may be the same or different.

The examples of the condensed aryl having 10 to 50 carbon atoms are 1-naphthyl, 2-naphthyl, 1-anthryl, 2-anthryl, 9-anthryl, 1-phenanthryl, 2-phenanthryl, 3-phenanthryl, 4-phenanthryl, 9-phenanthryl, 1-pyrenyl, 2-pyrenyl, 4-pyrenyl, 1-perylenyl, 2-perylenyl, 1-chrysenyl, 2-chrysenyl, 3-chrysenyl, 5-chrysenyl, 6-chrysenyl, 1-triphenylenyl, 2-triphenylenyl and 2-fluorenyl. Optional hydrogens in the above condensed aryl having a carbon number of 10 to 50 may be substituted with alkyl having a carbon number of 1 to 24, cycloalkyl having a carbon number of 3 to 24 or aryl having a carbon number of 6 to 24.

The examples of the condensed aryl having 10 to 50 carbon atoms in which optional hydrogens are replaced by the alkyl having 1 to 24 carbon atoms are 4-methyl-1-naphthyl, 4-tert-butyl-1-naphthyl, 6-methyl-2-naphthyl, 6-tert-butyl-2-naphthyl, 4-methyl-1-anthryl, 4-tert-butyl-1-anthryl, 10-methyl-9-anthryl, 10-tert-butyl-9-anthryl and 9,9-dimethyl-2-fluorenyl.

The examples of the condensed aryl having 10 to 50 carbon atoms in which optional hydrogens are replaced by with the cycloalkyl having 3 to 24 carbon atoms are 4-cyclohexyl-1-naphthyl, 6-cyclohexyl-2-naphthyl, 4-cyclohexyl-1-anthryl, 10-cyclohexyl-9-anthryl and 9,9-dicyclohexyl-2-fluorenyl.

The examples of the condensed aryl having 10 to 50 carbon atoms in which optional hydrogens are replaced by the aryl having a carbon number of 6 to 24 carbon atoms are 4-phenyl-1-naphthyl, 6-phenyl-2-naphthyl, 6-(2-naphthyl)-2-naphthyl, 6-(1-naphthyl)-2-naphthyl, 4-(2-naphthyl)-1-naphthyl, 4-(1-naphthyl)-1-naphthyl and 9,9-diphenyl-2-fluorenyl.

The examples of the heteroaryl are 1-pyrroryl, 2-pyrroryl, 3-pyrroryl, 2-pyridyl, 3-pyridyl, 4-pyridyl, 2,2′-bipyridyl-6-yl, 2,3′-bipyridyl-6-yl, 2,4′-bipyridyl-6-yl, 3,2′-bipyridyl-6-yl, 3,3′-bipyridyl-6-yl, 3,4′-bipyridyl-6-yl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 1,7-phenanthroline-2-yl, 1,7-phenanthroline-3-yl, 1,7-phenanthroline-4-yl, 1,7-phenanthroline-5-yl, 1,7-phenanthroline-6-yl, 1,7-phenanthroline-8-yl, 1,7-phenanthroline-9-yl, 1,7-phenanthroline-10-yl, 1,8-phenanthroline-2-yl, 1,8-phenanthroline-3-yl, 1,8-phenanthroline-4-yl, 1,8-phenanthroline-5-yl, 1,8-phenanthroline-6-yl, 1,8-phenanthroline-7-yl, 1,8-phenanthroline-9-yl, 1,8-phenanthroline-10-yl, 1,9-phenanthroline-2-yl, 1,9-phenanthroline-3-yl, 1,9-phenanthroline-4-yl, 1,9-phenanthroline-5-yl, 1,9-phenanthroline-6-yl, 1,9-phenanthroline-7-yl, 1,9-phenanthroline-8-yl, 1,9-phenanthroline-10-yl, 1,10-phenanthroline-2-yl, 1,10-phenanthroline-3-yl, 1,10-phenanthroline-4-yl, 1,10-phenanthroline-5-yl, 2,9-phenanthroline-1-yl, 2,9-phenanthroline-3-yl, 2,9-phenanthroline-4-yl, 2,9-phenanthroline-5-yl, 2,8-phenanthroline-1-yl, 2,8-phenanthroline-3-yl, 2,8-phenanthroline-4-yl, 2,8-phenanthroline-5-yl, 2,8-phenanthroline-6-yl, 2,8-phenanthroline-7-yl, 2,8-phenanthroline-9-yl, 2,8-phenanthroline-10-yl, 2,7-phenanthroline-1-yl, 2,7-phenanthroline-3-yl, 2,7-phenanthroline-4-yl, 2,7-phenanthroline-5-yl, 2,7-phenanthroline-6-yl, 2,7-phenanthroline-8-yl, 2,7-phenanthroline-9-yl, 2,7-phenanthroline-10-yl, 1-phenazinyl, 2-phenazinyl, 1-phenothiazinyl, 2-phenothiazinyl, 3-phenothiazinyl, 4-phenothiazinyl, 10-phenothiazinyl, 1-phenoxazinyl, 2-phenoxazinyl, 3-phenoxazinyl, 4-phenoxazinyl, 10-phenoxazinyl, 3-furazanyl, 2-thienyl, 3-thienyl, 2-benzothienyl, 3-benzothienyl, 4-benzothienyl, 5-benzothienyl, 6-benzothienyl, 7-benzothienyl, 1-isobenzothienyl, 3-isobenzothienyl, 4-isobenzothienyl, 5-isobenzothienyl, 6-isobenzothienyl and 7-isobenzothienyl.

Optional hydrogens in the above heteroaryl may be replaced by alkyl having 1 to 24 carbon atoms, cycloalkyl having 3 to 24 carbon atoms or aryl having 6 to 24 carbon atoms.

The examples of the heteroaryl in which optional hydrogens are replaced by the alkyl having 1 to 24 carbon are 5-methyl-2-thienyl, 5-methyl-3-thienyl, 2,5-dimethyl-3-thienyl, 3,4,5-trimethyl-2-thienyl, 3-methyl-2-benzothienyl, 2-methyl-3-benzothienyl, 2-methylpyrrole-1-yl, 2,5-dimethylpyrrole-1-yl, 2-methyl-1-indolyl, 2-tert-butyl-1-indolyl, 3-methyl-9-carbazolyl, 3,6-dimethyl-9-carbazolyl, 3,6-di-tert-butyl-9-carbazolyl and 9-methyl-3-carbazolyl.

The examples of the heteroaryl in which optional hydrogens are replaced by the cycloalkyl having 3 to 24 carbon atoms are 5-cyclohexyl-2-thienyl, 3-cyclohexyl-2-benzothienyl, 2-cyclohexyl-3-benzothienyl, 3-cyclohexyl-9-carbazolyl, 3,6-dicyclohexyl-9-carbazolyl and 9-cyclohexyl-3-carbazolyl.

The examples of the heteroaryl in which optional hydrogens are replaced by the aryl having 6 to 24 carbon atoms are 5-phenyl-2-thienyl, 5-(1-naphthyl)-2-thienyl, 5-(2-naphthyl)-2-thienyl, 5-phenyl-3-thienyl, 2,5-diphenyl-3-thienyl, 2-phenyl-5-(1-naphthyl)-3-thienyl, 2-phenyl-5-(2-naphthyl)-3-thienyl, 3,4,5-triphenyl-2-thienyl, 3,4-diphenyl-5-(1-naphthyl)-2-thienyl, 3,4-diphenyl-5-(2-naphthyl)-2-thienyl, 3-phenyl-2-benzothienyl, 3-(1-naphthyl)-2-benzothienyl, 3-(2-naphthyl)-2-benzothienyl, 2-phenyl-3-benzothienyl, 3-phenyl-9-carbazolyl, 3-(1-naphthyl)-9-carbazolyl, 3-(2-naphthyl)-9-carbazolyl, 3,6-diphenyl-9-carbazolyl, 3,6-di(1-naphthyl)-9-carbazolyl, 3,6-di(2-naphthyl)-9-carbazolyl, 3,6-di(4-tert-butylphenyl)-9-carbazolyl, 9-phenyl-3-carbazolyl, 9-(1-naphthyl)-3-carbazolyl and 9-(2-naphthyl)-3-carbazolyl.

The preferred examples of Ar² and Ar³ are phenyl, 2-biphenylyl, 3-biphenylyl, 4-biphenylyl, 2,6-dimethylphenyl, 2,4,6-trimethylphenyl, 4-tert-butylphenyl, 2,4-di-tert-butylphenyl, m-terphenyl-4′-yl, m-terphenyl-5′-yl, p-terphenyl-2′-yl, m-terphenyl-2-yl, m-terphenyl-3-yl, o-terphenyl-2-yl, o-terphenyl-3-yl, p-quaterphenyl-3-yl, m-quaterphenyl-3-yl, o-quaterphenyl-2-yl, 3,5-di(2-naphthyl)phenyl, 3,5-di(1-naphthyl)phenyl, 4-(9-carbazolyl)phenyl, 3,5-di(9-carbazolyl)phenyl, 1-naphthyl, 2-naphthyl, 4-phenyl-1-naphthyl, 6-phenyl-2-naphthyl, 4-(2-naphthyl)-1-naphthyl, 6-(2-naphthyl)-2-naphthyl, 4-(9-carbazolyl)-1-naphthyl, 6-(9-carbazolyl)-2-naphthyl, 9-phenanthryl, 2-triphenylenyl, 9,9-dimethyl-2-fluorenyl, 9,9-diphenyl-2-fluorenyl, 5-phenyl-2-thienyl, 2,5-diphenyl-3-thienyl, 3,4,5-triphenyl-2-thienyl, 2-benzothienyl, 3-phenyl-2-benzothienyl, 2-phenyl-3-benzothienyl and 9-carbazolyl.

The more preferred examples of Ar² and Ar³ are phenyl, 2-biphenylyl, 3-biphenylyl, 4-biphenylyl, 4-tert-butylphenyl, m-terphenyl-5′-yl, 4-(9-carbazolyl)phenyl, p-quaterphenyl-3-yl, m-quaterphenyl-3-yl, o-quaterphenyl-2-yl, 3,5-di(2-naphthyl)phenyl, 1-naphthyl, 2-naphthyl, 4-phenyl-1-naphthyl, 6-(m-terphenyl-5′-yl)-2-naphthyl, 6-(2-naphthyl)-2-naphthyl, 4-(9-carbazolyl)-1-naphthyl, 9-phenanthryl, 2-benzothienyl and 3-phenyl-2-benzothienyl.

If hydrogens in positions adjacent to atoms bonded to anthracene in Ar² and Ar³ are substituted with substituents, emission wavelength of blue color originating in the fundamental skeleton can be maintained, and it is suited to blue color emission. If hydrogens in the other positions are substituted, the compound is increased in rigidity and excellent in heat resistance. The emission material meeting the object can be obtained by suitably selecting the number of the substituents and the positions thereof considering emission wavelength and heat resistance expected to the emission material based on the design of the device.

Compounds of (1-1) to (1-1426) which are the specific examples of the emission material (1) of the present invention are shown in the following Table 2-1 to Table 2-31. Codes used in Table 2-1 to Table 2-31 are shown in Table 1-1 to Table 1-5. For example, the compound (1-15) shown in Table 2-1, the compound (1-412) shown in Table 2-9, the compound (1-419) shown in Table 2-10 and the compound (1-606) shown in Table 2-14 have the following structures. However, the present invention shall not be restricted by disclosing these specific structures.

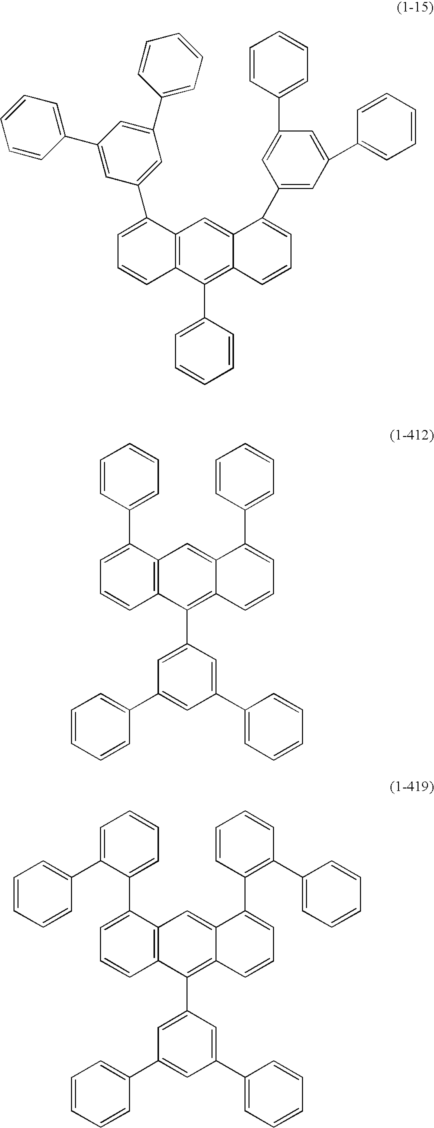

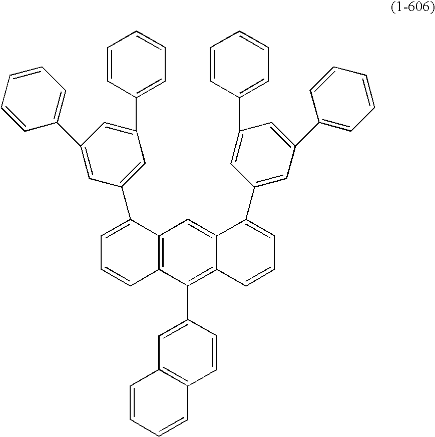

TABLE 1

| sym- | |
|---|---|
| bol | structural formula |
| P1 | 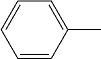 |
| P2 | 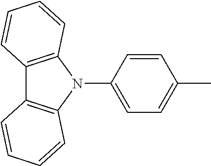 |
| P3 | 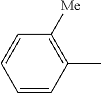 |
| P4 | 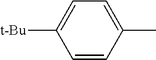 |
| P5 | 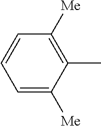 |
| P6 | 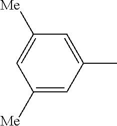 |
| P7 | 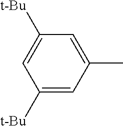 |
| P8 | 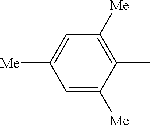 |
| BP1 | 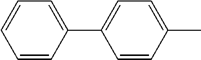 |
| BP2 | 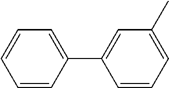 |
| BP3 | 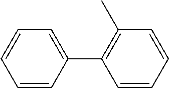 |
| BP4 | 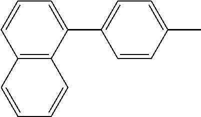 |
| BP5 | 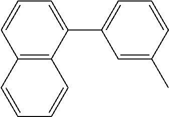 |
| BP6 | 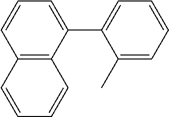 |
| BP7 | 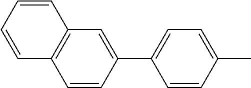 |
| BP8 | 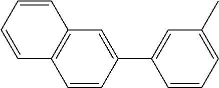 |
| BP9 | 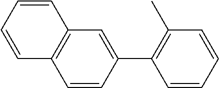 |
| TP1 | 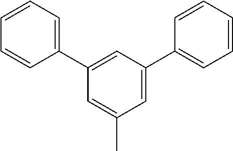 |
| TP2 | 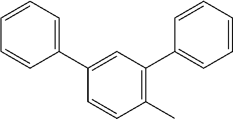 |
| TP3 | 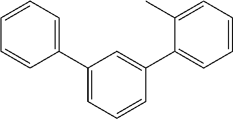 |
| TP4 | 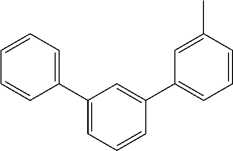 |
| TP5 | 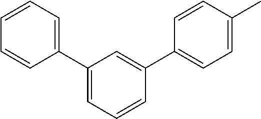 |
| TP6 | 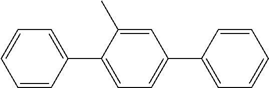 |
| TP7 | 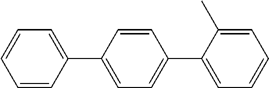 |
| TP8 | 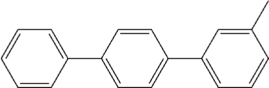 |
| TP9 | 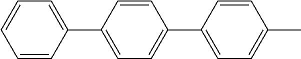 |
| TP10 | 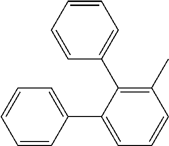 |
| TP11 | 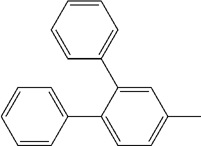 |
| TP12 | 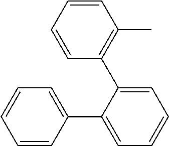 |
| TP13 | 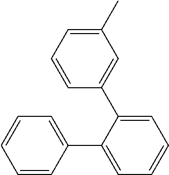 |
| TP14 | 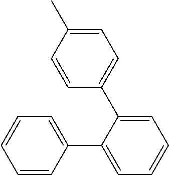 |
| TP15 | 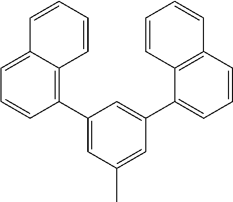 |
| TP16 | 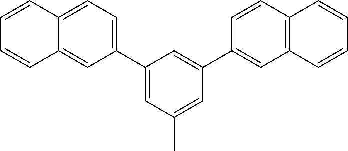 |
| TP17 | 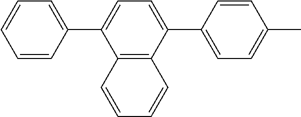 |
| TP18 | 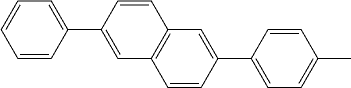 |
| TP19 | 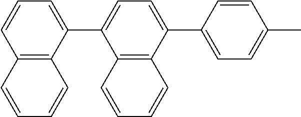 |
| QP1 | 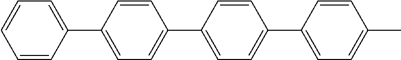 |
| QP2 | 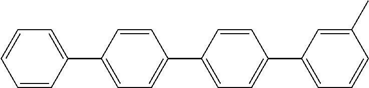 |
| QP3 | 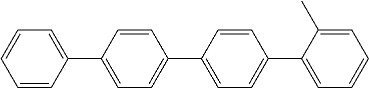 |
| QP4 | 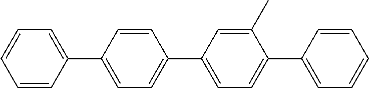 |
| QP5 | 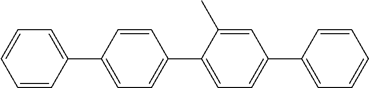 |
| QP6 | 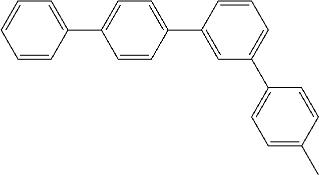 |
| QP7 | 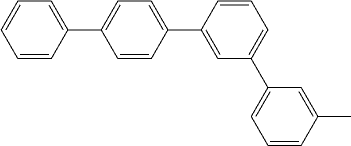 |
| QP8 | 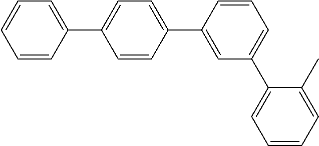 |
| QP9 | 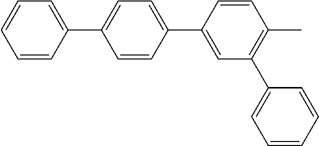 |
| QP10 | 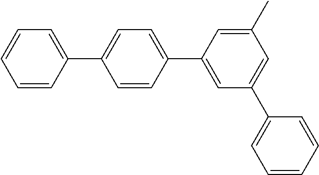 |
| QP11 | 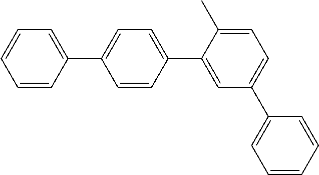 |
| QP12 | 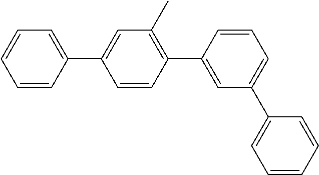 |
| QP13 | 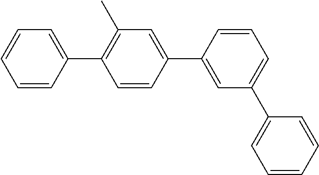 |
| QP14 | 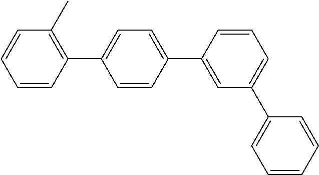 |
| QP15 | 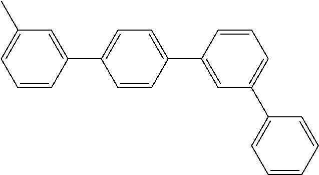 |
| QP16 | 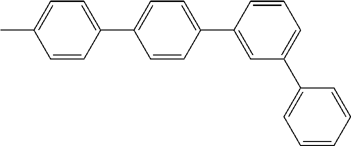 |
| QP17 | 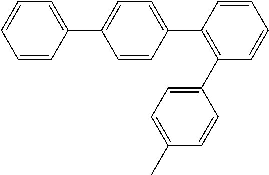 |
| QP18 | 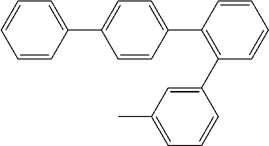 |
| QP19 | 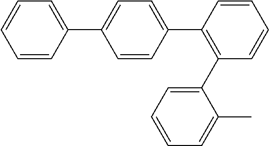 |
| QP20 | 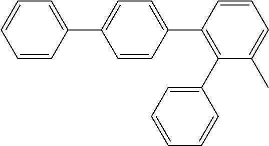 |
| QP21 | 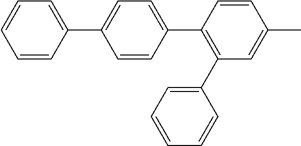 |
| QP22 | 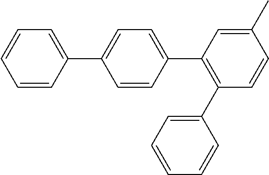 |
| QP23 | 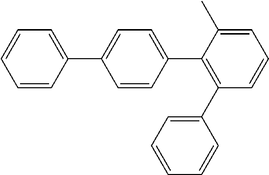 |
| QP24 | 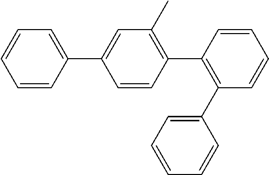 |
| QP25 | 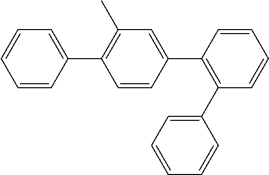 |
| QP26 | 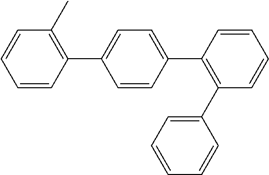 |
| QP27 | 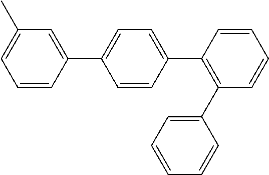 |
| QP28 | 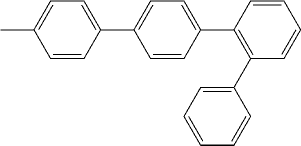 |
| QP29 | 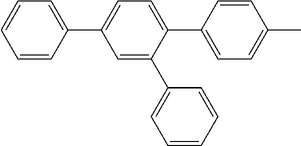 |
| QP30 | 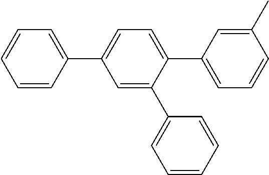 |
| QP31 | 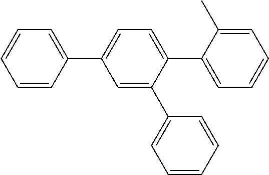 |
| QP32 | 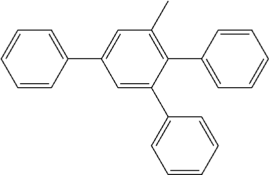 |
| QP33 | 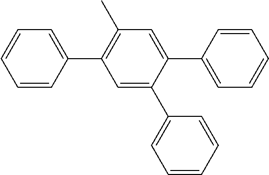 |
| QP34 | 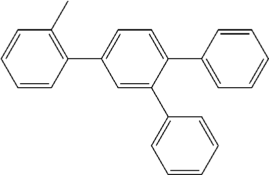 |
| QP35 | 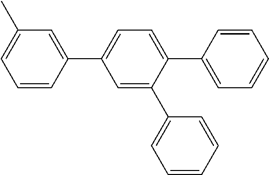 |
| QP36 | 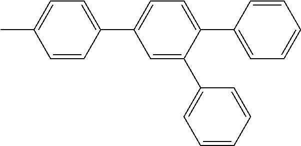 |
| QP37 | 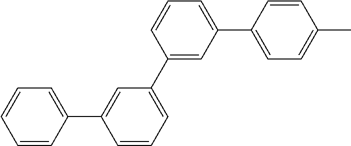 |
| QP38 | 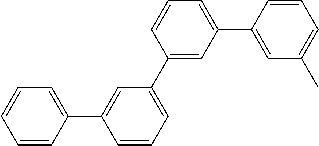 |
| QP39 | 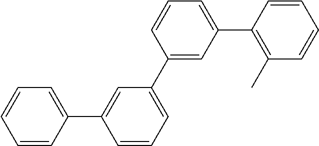 |
| QP40 | 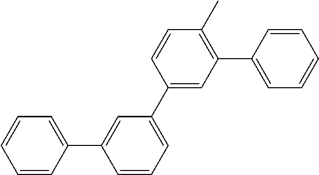 |
| QP41 | 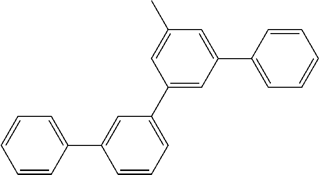 |
| QP42 | 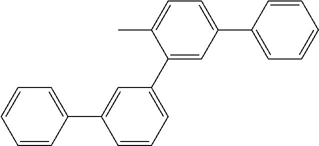 |
| QP43 | 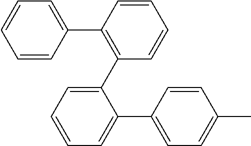 |
| QP44 | 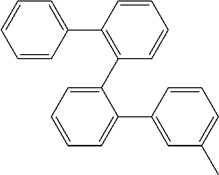 |
| QP45 | 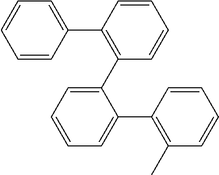 |
| QP46 | 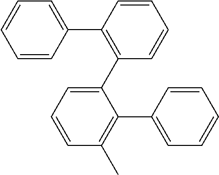 |
| QP47 | 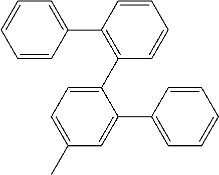 |
| QP48 | 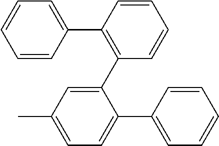 |
| QP49 | 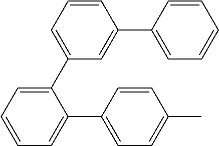 |
| QP50 | 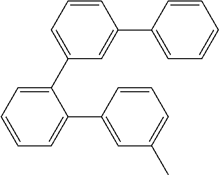 |
| QP51 | 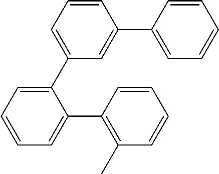 |
| QP52 | 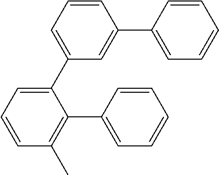 |
| QP53 | 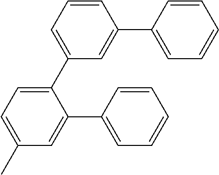 |
| QP54 | 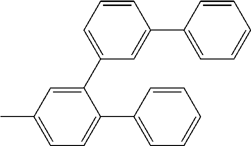 |
| QP55 | 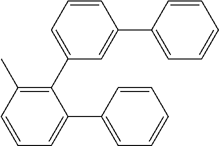 |
| QP56 | 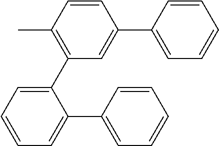 |
| QP57 | 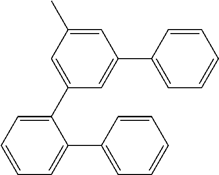 |
| QP58 | 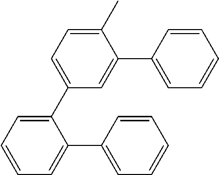 |
| QP59 | 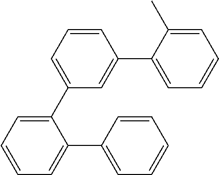 |
| QP60 | 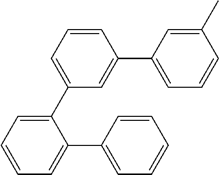 |
| QP61 | 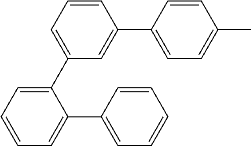 |
| QP62 | 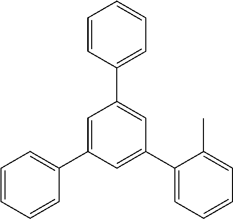 |
| QP63 | 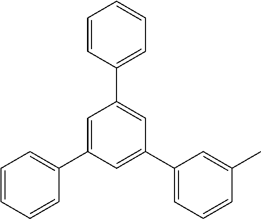 |
| QP64 | 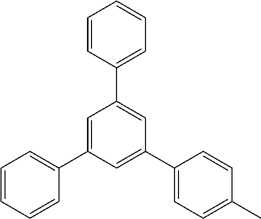 |
| QP65 | 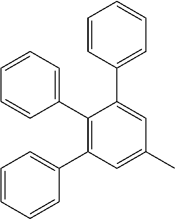 |
| QP66 | 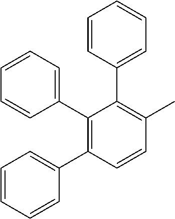 |
| NP1 | 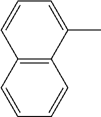 |
| NP2 | 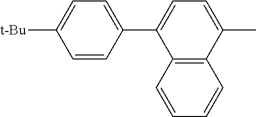 |
| NP3 | 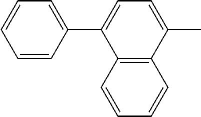 |
| NP4 | 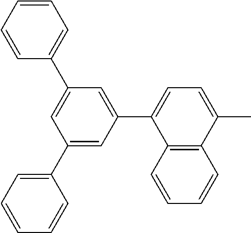 |
| NP5 | 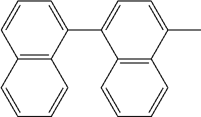 |
| NP6 | 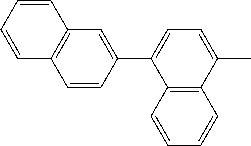 |
| NP7 | 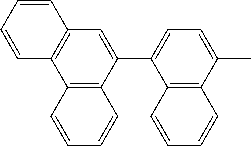 |
| NP8 | 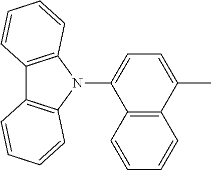 |
| NP9 | 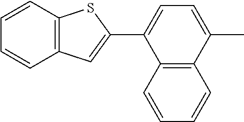 |
| NP10 | 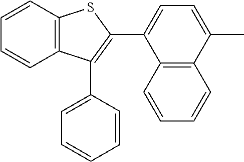 |
| NP11 | 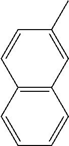 |
| NP12 | 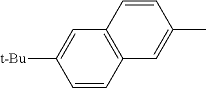 |
| NP13 | 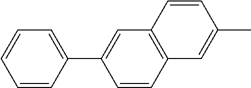 |
| NP14 | 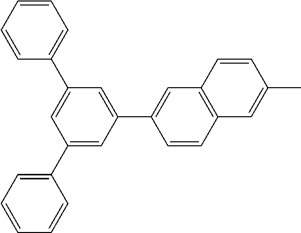 |
| NP15 | 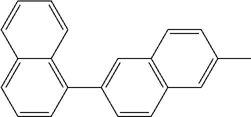 |
| NP16 | 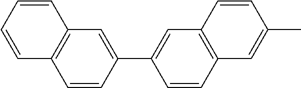 |
| NP17 | 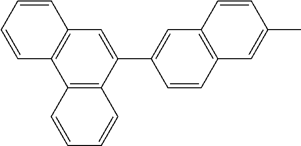 |
| NP18 | 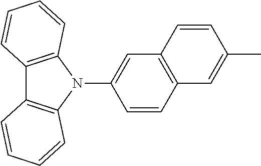 |
| NP19 | 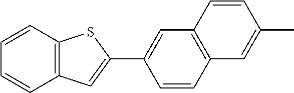 |
| NP20 | 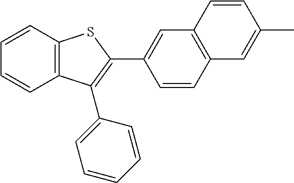 |
| PN1 | 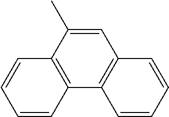 |
| CS1 | 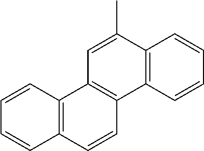 |
| TPL1 | 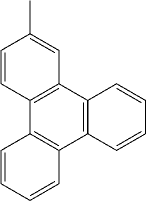 |
| FL1 | 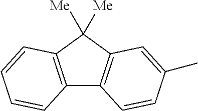 |
| FL2 | 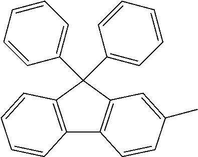 |
| TH1 | 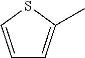 |
| TH2 | 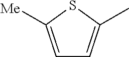 |
| TH3 | 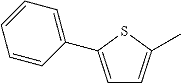 |
| TH4 | 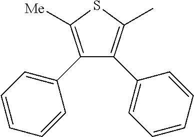 |
| TH5 | 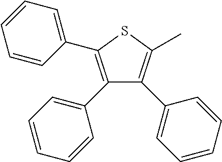 |
| BT1 | 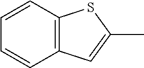 |
| BT2 | 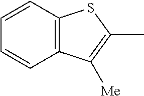 |
| BT3 | 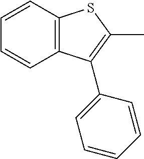 |
| BT4 | 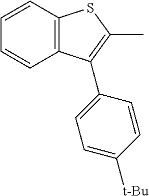 |
| BT5 | 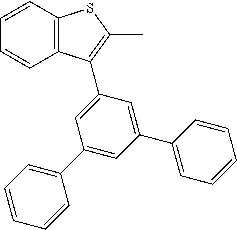 |
| BT6 | 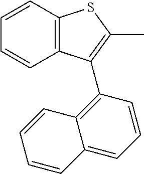 |
| BT7 | 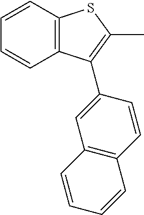 |
| BT8 | 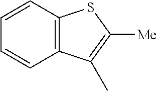 |
| BT9 | 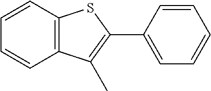 |
| BT10 | 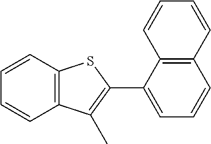 |
| BT11 | 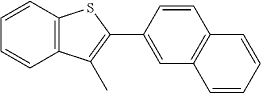 |
| CZ1 | 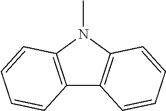 |
| CZ2 | 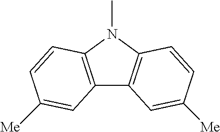 |
| CZ3 | 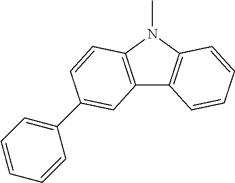 |
| CZ4 | 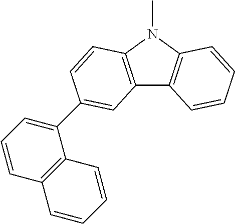 |
| CZ5 | 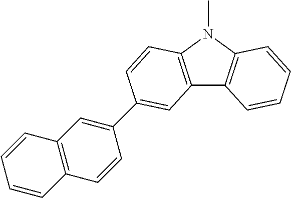 |
| CZ6 | 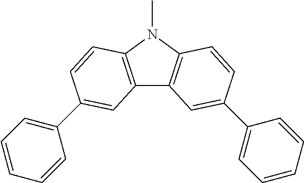 |
| CZ7 | 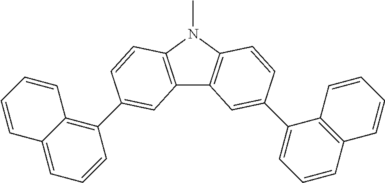 |
| CZ8 | 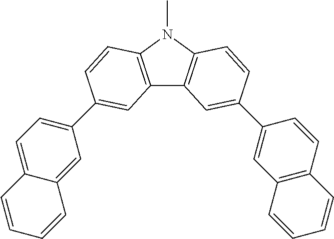 |
| CZ9 | 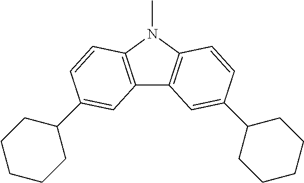 |
| IN1 | 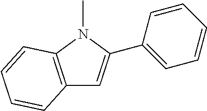 |
| PY1 | 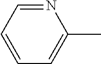 |
| PY2 | 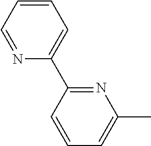 |
| PY3 | 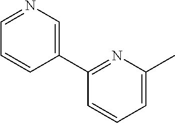 |
| QN1 | 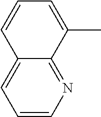 |
| QN2 | 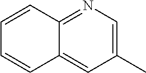 |
| QN3 | 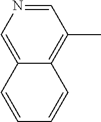 |
| CY | 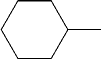 |
| PO | 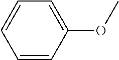 |
| TPM | 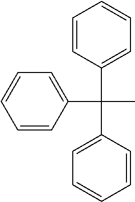 |

| | | | | | | | | | | |
|---|---|---|---|---|---|---|---|---|---|---|
| No. | Ar¹ | Ar² | Ar³ | R¹ | R² | R³ | R⁴ | R⁵ | R⁶ | R⁷ |

Table 2-1

| | | | | | | | | | | |
|---|---|---|---|---|---|---|---|---|---|---|
| 1-1 | P1 | P2 | P2 | H | H | H | H | H | H | H |
| 1-2 | P1 | P4 | P4 | H | H | H | H | H | H | H |
| 1-3 | P1 | P5 | P5 | H | H | H | H | H | H | H |
| 1-4 | P1 | P6 | P6 | H | H | H | H | H | H | H |
| 1-5 | P1 | P8 | P8 | H | H | H | H | H | H | H |
| 1-6 | P1 | BP1 | BP1 | H | H | H | H | H | H | H |
| 1-7 | P1 | BP2 | BP2 | H | H | H | H | H | H | H |
| 1-8 | P1 | BP3 | BP3 | H | H | H | H | H | H | H |
| 1-9 | P1 | BP4 | BP4 | H | H | H | H | H | H | H |
| 1-10 | P1 | BP5 | BP5 | H | H | H | H | H | H | H |
| 1-11 | P1 | BP6 | BP6 | H | H | H | H | H | H | H |
| 1-12 | P1 | BP7 | BP7 | H | H | H | H | H | H | H |
| 1-13 | P1 | BP8 | BP8 | H | H | H | H | H | H | H |
| 1-14 | P1 | BP9 | BP9 | H | H | H | H | H | H | H |
| 1-15 | P1 | TP1 | TP1 | H | H | H | H | H | H | H |
| 1-16 | P1 | TP2 | TP2 | H | H | H | H | H | H | H |
| 1-17 | P1 | TP3 | TP3 | H | H | H | H | H | H | H |
| 1-18 | P1 | TP4 | TP4 | H | H | H | H | H | H | H |
| 1-19 | P1 | TP5 | TP5 | H | H | H | H | H | H | H |
| 1-20 | P1 | TP6 | TP6 | H | H | H | H | H | H | H |
| 1-21 | P1 | TP7 | TP7 | H | H | H | H | H | H | H |
| 1-22 | P1 | TP8 | TP8 | H | H | H | H | H | H | H |
| 1-23 | P1 | TP9 | TP9 | H | H | H | H | H | H | H |
| 1-24 | P1 | TP10 | TP10 | H | H | H | H | H | H | H |
| 1-25 | P1 | TP11 | TP11 | H | H | H | H | H | H | H |
| 1-26 | P1 | TP12 | TP12 | H | H | H | H | H | H | H |
| 1-27 | P1 | TP13 | TP13 | H | H | H | H | H | H | H |
| 1-28 | P1 | TP14 | TP14 | H | H | H | H | H | H | H |
| 1-29 | P1 | TP15 | TP15 | H | H | H | H | H | H | H |
| 1-30 | P1 | TP16 | TP16 | H | H | H | H | H | H | H |
| 1-31 | P1 | TP17 | TP17 | H | H | H | H | H | H | H |
| 1-32 | P1 | TP18 | TP18 | H | H | H | H | H | H | H |
| 1-33 | P1 | TP19 | TP19 | H | H | H | H | H | H | H |
| 1-34 | P1 | QP1 | QP1 | H | H | H | H | H | H | H |
| 1-35 | P1 | QP2 | QP2 | H | H | H | H | H | H | H |
| 1-36 | P1 | QP3 | QP3 | H | H | H | H | H | H | H |
| 1-37 | P1 | QP4 | QP4 | H | H | H | H | H | H | H |
| 1-38 | P1 | QP5 | QP5 | H | H | H | H | H | H | H |
| 1-39 | P1 | QP6 | QP6 | H | H | H | H | H | H | H |
| 1-40 | P1 | QP7 | QP7 | H | H | H | H | H | H | H |
| 1-41 | P1 | QP8 | QP8 | H | H | H | H | H | H | H |
| 1-42 | P1 | QP9 | QP9 | H | H | H | H | H | H | H |
| 1-43 | P1 | QP10 | QP10 | H | H | H | H | H | H | H |
| 1-44 | P1 | QP11 | QP11 | H | H | H | H | H | H | H |
| 1-45 | P1 | QP12 | QP12 | H | H | H | H | H | H | H |
| 1-46 | P1 | QP13 | QP13 | H | H | H | H | H | H | H |

Table 2-2

| | | | | | | | | | | |
|---|---|---|---|---|---|---|---|---|---|---|
| 1-47 | P1 | QP14 | QP14 | H | H | H | H | H | H | H |
| 1-48 | P1 | QP15 | QP15 | H | H | H | H | H | H | H |
| 1-49 | P1 | QP16 | QP16 | H | H | H | H | H | H | H |
| 1-50 | P1 | QP17 | QP17 | H | H | H | H | H | H | H |
| 1-51 | P1 | QP18 | QP18 | H | H | H | H | H | H | H |
| 1-52 | P1 | QP19 | QP19 | H | H | H | H | H | H | H |
| 1-53 | P1 | QP20 | QP20 | H | H | H | H | H | H | H |
| 1-54 | P1 | QP21 | QP21 | H | H | H | H | H | H | H |
| 1-55 | P1 | QP22 | QP22 | H | H | H | H | H | H | H |
| 1-56 | P1 | QP23 | QP23 | H | H | H | H | H | H | H |
| 1-57 | P1 | QP24 | QP24 | H | H | H | H | H | H | H |
| 1-58 | P1 | QP25 | QP25 | H | H | H | H | H | H | H |
| 1-59 | P1 | QP26 | QP26 | H | H | H | H | H | H | H |
| 1-60 | P1 | QP27 | QP27 | H | H | H | H | H | H | H |
| 1-61 | P1 | QP28 | QP28 | H | H | H | H | H | H | H |
| 1-62 | P1 | QP29 | QP29 | H | H | H | H | H | H | H |
| 1-63 | P1 | QP30 | QP30 | H | H | H | H | H | H | H |
| 1-64 | P1 | QP31 | QP31 | H | H | H | H | H | H | H |
| 1-65 | P1 | QP32 | QP32 | H | H | H | H | H | H | H |
| 1-66 | P1 | QP33 | QP33 | H | H | H | H | H | H | H |
| 1-67 | P1 | QP34 | QP34 | H | H | H | H | H | H | H |
| 1-68 | P1 | QP35 | QP35 | H | H | H | H | H | H | H |
| 1-69 | P1 | QP36 | QP36 | H | H | H | H | H | H | H |
| 1-70 | P1 | QP37 | QP37 | H | H | H | H | H | H | H |
| 1-71 | P1 | QP38 | QP38 | H | H | H | H | H | H | H |
| 1-72 | P1 | QP39 | QP39 | H | H | H | H | H | H | H |
| 1-73 | P1 | QP40 | QP40 | H | H | H | H | H | H | H |
| 1-74 | P1 | QP41 | QP41 | H | H | H | H | H | H | H |
| 1-75 | P1 | QP42 | QP42 | H | H | H | H | H | H | H |
| 1-76 | P1 | QP43 | QP43 | H | H | H | H | H | H | H |
| 1-77 | P1 | QP44 | QP44 | H | H | H | H | H | H | H |
| 1-78 | P1 | QP45 | QP45 | H | H | H | H | H | H | H |
| 1-79 | P1 | QP46 | QP46 | H | H | H | H | H | H | H |
| 1-80 | P1 | QP47 | QP47 | H | H | H | H | H | H | H |
| 1-81 | P1 | QP48 | QP48 | H | H | H | H | H | H | H |
| 1-82 | P1 | QP49 | QP49 | H | H | H | H | H | H | H |
| 1-83 | P1 | QP50 | QP50 | H | H | H | H | H | H | H |
| 1-84 | P1 | QP51 | QP51 | H | H | H | H | H | H | H |
| 1-85 | P1 | QP52 | QP52 | H | H | H | H | H | H | H |
| 1-86 | P1 | QP53 | QP53 | H | H | H | H | H | H | H |
| 1-87 | P1 | QP54 | QP54 | H | H | H | H | H | H | H |
| 1-88 | P1 | QP55 | QP55 | H | H | H | H | H | H | H |
| 1-89 | P1 | QP56 | QP56 | H | H | H | H | H | H | H |
| 1-90 | P1 | QP57 | QP57 | H | H | H | H | H | H | H |
| 1-91 | P1 | QP58 | QP58 | H | H | H | H | H | H | H |
| 1-92 | P1 | QP59 | QP59 | H | H | H | H | H | H | H |

Table 2-3

| | | | | | | | | | | |
|---|---|---|---|---|---|---|---|---|---|---|
| 1-93 | P1 | QP60 | QP60 | H | H | H | H | H | H | H |
| 1-94 | P1 | QP61 | QP61 | H | H | H | H | H | H | H |
| 1-95 | P1 | QP62 | QP62 | H | H | H | H | H | H | H |
| 1-96 | P1 | QP63 | QP63 | H | H | H | H | H | H | H |
| 1-97 | P1 | QP64 | QP64 | H | H | H | H | H | H | H |
| 1-98 | P1 | QP65 | QP65 | H | H | H | H | H | H | H |
| 1-99 | P1 | QP66 | QP66 | H | H | H | H | H | H | H |
| 1-100 | P1 | NP1 | NP1 | H | H | H | H | H | H | H |
| 1-101 | P1 | NP2 | NP2 | H | H | H | H | H | H | H |
| 1-102 | P1 | NP3 | NP3 | H | H | H | H | H | H | H |
| 1-103 | P1 | NP4 | NP4 | H | H | H | H | H | H | H |
| 1-104 | P1 | NP5 | NP5 | H | H | H | H | H | H | H |
| 1-105 | P1 | NP6 | NP6 | H | H | H | H | H | H | H |
| 1-106 | P1 | NP7 | NP7 | H | H | H | H | H | H | H |
| 1-107 | P1 | NP8 | NP8 | H | H | H | H | H | H | H |
| 1-108 | P1 | NP9 | NP9 | H | H | H | H | H | H | H |
| 1-109 | P1 | NP10 | NP10 | H | H | H | H | H | H | H |
| 1-110 | P1 | NP11 | NP11 | H | H | H | H | H | H | H |
| 1-111 | P1 | NP12 | NP12 | H | H | H | H | H | H | H |
| 1-112 | P1 | NP13 | NP13 | H | H | H | H | H | H | H |
| 1-113 | P1 | NP14 | NP14 | H | H | H | H | H | H | H |
| 1-114 | P1 | NP15 | NP15 | H | H | H | H | H | H | H |
| 1-115 | P1 | NP16 | NP16 | H | H | H | H | H | H | H |
| 1-116 | P1 | NP17 | NP17 | H | H | H | H | H | H | H |
| 1-117 | P1 | NP18 | NP18 | H | H | H | H | H | H | H |
| 1-118 | P1 | NP19 | NP19 | H | H | H | H | H | H | H |
| 1-119 | P1 | NP20 | NP20 | H | H | H | H | H | H | H |
| 1-120 | P1 | PN1 | PN1 | H | H | H | H | H | H | H |
| 1-121 | P1 | CS1 | CS1 | H | H | H | H | H | H | H |
| 1-122 | P1 | TPL1 | TPL1 | H | H | H | H | H | H | H |
| 1-123 | P1 | FL1 | FL1 | H | H | H | H | H | H | H |
| 1-124 | P1 | FL2 | FL2 | H | H | H | H | H | H | H |
| 1-125 | P1 | TH1 | TH1 | H | H | H | H | H | H | H |
| 1-126 | P1 | TH2 | TH2 | H | H | H | H | H | H | H |
| 1-127 | P1 | TH3 | TH3 | H | H | H | H | H | H | H |
| 1-128 | P1 | TH4 | TH4 | H | H | H | H | H | H | H |
| 1-129 | P1 | TH5 | TH5 | H | H | H | H | H | H | H |
| 1-130 | P1 | BT1 | BT1 | H | H | H | H | H | H | H |
| 1-131 | P1 | BT2 | BT2 | H | H | H | H | H | H | H |
| 1-132 | P1 | BT3 | BT3 | H | H | H | H | H | H | H |
| 1-133 | P1 | BT4 | BT4 | H | H | H | H | H | H | H |
| 1-134 | P1 | BT5 | BT5 | H | H | H | H | H | H | H |
| 1-135 | P1 | BT6 | BT6 | H | H | H | H | H | H | H |
| 1-136 | P1 | BT7 | BT7 | H | H | H | H | H | H | H |
| 1-137 | P1 | BT8 | BT8 | H | H | H | H | H | H | H |
| 1-138 | P1 | BT9 | BT9 | H | H | H | H | H | H | H |

Table 2-4

| | | | | | | | | | | |
|---|---|---|---|---|---|---|---|---|---|---|
| 1-139 | P1 | BT10 | BT10 | H | H | H | H | H | H | H |
| 1-140 | P1 | BT11 | BT11 | H | H | H | H | H | H | H |
| 1-141 | P1 | CZ1 | CZ1 | H | H | H | H | H | H | H |
| 1-142 | P1 | CZ2 | CZ2 | H | H | H | H | H | H | H |
| 1-143 | P1 | CZ3 | CZ3 | H | H | H | H | H | H | H |
| 1-144 | P1 | CZ4 | CZ4 | H | H | H | H | H | H | H |
| 1-145 | P1 | CZ5 | CZ5 | H | H | H | H | H | H | H |
| 1-146 | P1 | CZ6 | CZ6 | H | H | H | H | H | H | H |
| 1-147 | P1 | CZ7 | CZ7 | H | H | H | H | H | H | H |
| 1-148 | P1 | CZ8 | CZ8 | H | H | H | H | H | H | H |
| 1-149 | P1 | CZ9 | CZ9 | H | H | H | H | H | H | H |
| 1-150 | P2 | P1 | P1 | H | H | H | H | H | H | H |
| 1-151 | P2 | P2 | P2 | H | H | H | H | H | H | H |
| 1-152 | P2 | P3 | P3 | H | H | H | H | H | H | H |
| 1-153 | P2 | P4 | P4 | H | H | H | H | H | H | H |
| 1-154 | P2 | P5 | P5 | H | H | H | H | H | H | H |
| 1-155 | P2 | P7 | P7 | H | H | H | H | H | H | H |
| 1-156 | P2 | P8 | P8 | H | H | H | H | H | H | H |
| 1-157 | P2 | BP1 | BP1 | H | H | H | H | H | H | H |
| 1-158 | P2 | BP2 | BP2 | H | H | H | H | H | H | H |
| 1-159 | P2 | BP3 | BP3 | H | H | H | H | H | H | H |
| 1-160 | P2 | BP4 | BP4 | H | H | H | H | H | H | H |
| 1-161 | P2 | BP7 | BP7 | H | H | H | H | H | H | H |
| 1-162 | P2 | BP8 | BP8 | H | H | H | H | H | H | H |
| 1-163 | P2 | TP1 | TP1 | H | H | H | H | H | H | H |
| 1-164 | P2 | TP3 | TP3 | H | H | H | H | H | H | H |
| 1-165 | P2 | TP4 | TP4 | H | H | H | H | H | H | H |
| 1-166 | P2 | TP5 | TP5 | H | H | H | H | H | H | H |
| 1-167 | P2 | TP6 | TP6 | H | H | H | H | H | H | H |
| 1-168 | P2 | TP7 | TP7 | H | H | H | H | H | H | H |
| 1-169 | P2 | TP8 | TP8 | H | H | H | H | H | H | H |
| 1-170 | P2 | TP9 | TP9 | H | H | H | H | H | H | H |
| 1-171 | P2 | TP12 | TP12 | H | H | H | H | H | H | H |
| 1-172 | P2 | TP13 | TP13 | H | H | H | H | H | H | H |
| 1-173 | P2 | TP14 | TP14 | H | H | H | H | H | H | H |
| 1-174 | P2 | TP15 | TP15 | H | H | H | H | H | H | H |
| 1-175 | P2 | TP16 | TP16 | H | H | H | H | H | H | H |
| 1-176 | P2 | TP17 | TP17 | H | H | H | H | H | H | H |
| 1-177 | P2 | TP18 | TP18 | H | H | H | H | H | H | H |
| 1-178 | P2 | TP19 | TP19 | H | H | H | H | H | H | H |
| 1-179 | P2 | QP38 | QP38 | H | H | H | H | H | H | H |
| 1-180 | P2 | QP39 | QP39 | H | H | H | H | H | H | H |
| 1-181 | P2 | QP44 | QP44 | H | H | H | H | H | H | H |
| 1-182 | P2 | QP45 | QP45 | H | H | H | H | H | H | H |
| 1-183 | P2 | QP63 | QP63 | H | H | H | H | H | H | H |
| 1-184 | P2 | QP64 | QP64 | H | H | H | H | H | H | H |

Table 2-5

| | | | | | | | | | | |
|---|---|---|---|---|---|---|---|---|---|---|
| 1-185 | P2 | NP1 | NP1 | H | H | H | H | H | H | H |
| 1-186 | P2 | NP2 | NP2 | H | H | H | H | H | H | H |
| 1-187 | P2 | NP3 | NP3 | H | H | H | H | H | H | H |
| 1-188 | P2 | NP4 | NP4 | H | H | H | H | H | H | H |
| 1-189 | P2 | NP5 | NP5 | H | H | H | H | H | H | H |
| 1-190 | P2 | NP6 | NP6 | H | H | H | H | H | H | H |
| 1-191 | P2 | NP7 | NP7 | H | H | H | H | H | H | H |
| 1-192 | P2 | NP8 | NP8 | H | H | H | H | H | H | H |
| 1-193 | P2 | NP11 | NP11 | H | H | H | H | H | H | H |
| 1-194 | P2 | NP12 | NP12 | H | H | H | H | H | H | H |
| 1-195 | P2 | NP13 | NP13 | H | H | H | H | H | H | H |
| 1-196 | P2 | NP14 | NP14 | H | H | H | H | H | H | H |
| 1-197 | P2 | NP15 | NP15 | H | H | H | H | H | H | H |
| 1-198 | P2 | NP16 | NP16 | H | H | H | H | H | H | H |
| 1-199 | P2 | NP17 | NP17 | H | H | H | H | H | H | H |
| 1-200 | P2 | NP18 | NP18 | H | H | H | H | H | H | H |
| 1-201 | P2 | PN1 | PN1 | H | H | H | H | H | H | H |
| 1-202 | P2 | FL1 | FL1 | H | H | H | H | H | H | H |
| 1-203 | P2 | TH1 | TH1 | H | H | H | H | H | H | H |
| 1-204 | P2 | TH2 | TH2 | H | H | H | H | H | H | H |
| 1-205 | P2 | TH3 | TH3 | H | H | H | H | H | H | H |
| 1-206 | P2 | BT1 | BT1 | H | H | H | H | H | H | H |
| 1-207 | P2 | BT3 | BT3 | H | H | H | H | H | H | H |
| 1-208 | P2 | BT6 | BT6 | H | H | H | H | H | H | H |
| 1-209 | P2 | BT7 | BT7 | H | H | H | H | H | H | H |
| 1-210 | P2 | BT9 | BT9 | H | H | H | H | H | H | H |
| 1-211 | P2 | CZ1 | CZ1 | H | H | H | H | H | H | H |
| 1-212 | P2 | CZ2 | CZ2 | H | H | H | H | H | H | H |
| 1-213 | P2 | CZ6 | CZ6 | H | H | H | H | H | H | H |
| 1-214 | P4 | P1 | P1 | H | H | H | H | H | H | H |
| 1-215 | P4 | P2 | P2 | H | H | H | H | H | H | H |
| 1-216 | P4 | P4 | P4 | H | H | H | H | H | H | H |
| 1-217 | P4 | P5 | P5 | H | H | H | H | H | H | H |
| 1-218 | P4 | P6 | P6 | H | H | H | H | H | H | H |
| 1-219 | P4 | P8 | P8 | H | H | H | H | H | H | H |
| 1-220 | P4 | BP1 | BP1 | H | H | H | H | H | H | H |
| 1-221 | P4 | BP2 | BP2 | H | H | H | H | H | H | H |
| 1-222 | P4 | BP3 | BP3 | H | H | H | H | H | H | H |
| 1-223 | P4 | BP4 | BP4 | H | H | H | H | H | H | H |
| 1-224 | P4 | BP7 | BP7 | H | H | H | H | H | H | H |
| 1-225 | P4 | TP1 | TP1 | H | H | H | H | H | H | H |
| 1-226 | P4 | TP2 | TP2 | H | H | H | H | H | H | H |
| 1-227 | P4 | TP4 | TP4 | H | H | H | H | H | H | H |
| 1-228 | P4 | TP5 | TP5 | H | H | H | H | H | H | H |
| 1-229 | P4 | TP6 | TP6 | H | H | H | H | H | H | H |
| 1-230 | P4 | TP8 | TP8 | H | H | H | H | H | H | H |

Table 2-6

| | | | | | | | | | | |
|---|---|---|---|---|---|---|---|---|---|---|
| 1-231 | P4 | TP9 | TP9 | H | H | H | H | H | H | H |
| 1-232 | P4 | TP12 | TP12 | H | H | H | H | H | H | H |
| 1-233 | P4 | TP13 | TP13 | H | H | H | H | H | H | H |
| 1-234 | P4 | TP14 | TP14 | H | H | H | H | H | H | H |
| 1-235 | P4 | TP15 | TP15 | H | H | H | H | H | H | H |
| 1-236 | P4 | TP16 | TP16 | H | H | H | H | H | H | H |
| 1-237 | P4 | TP17 | TP17 | H | H | H | H | H | H | H |
| 1-238 | P4 | TP18 | TP18 | H | H | H | H | H | H | H |
| 1-239 | P4 | TP19 | TP19 | H | H | H | H | H | H | H |
| 1-240 | P4 | QP38 | QP38 | H | H | H | H | H | H | H |
| 1-241 | P4 | QP39 | QP39 | H | H | H | H | H | H | H |
| 1-242 | P4 | QP44 | QP44 | H | H | H | H | H | H | H |
| 1-243 | P4 | QP45 | QP45 | H | H | H | H | H | H | H |
| 1-244 | P4 | QP63 | QP63 | H | H | H | H | H | H | H |
| 1-245 | P4 | QP64 | QP64 | H | H | H | H | H | H | H |
| 1-246 | P4 | NP1 | NP1 | H | H | H | H | H | H | H |
| 1-247 | P4 | NP2 | NP2 | H | H | H | H | H | H | H |
| 1-248 | P4 | NP3 | NP3 | H | H | H | H | H | H | H |
| 1-249 | P4 | NP4 | NP4 | H | H | H | H | H | H | H |
| 1-250 | P4 | NP5 | NP5 | H | H | H | H | H | H | H |
| 1-251 | P4 | NP6 | NP6 | H | H | H | H | H | H | H |
| 1-252 | P4 | NP7 | NP7 | H | H | H | H | H | H | H |
| 1-253 | P4 | NP8 | NP8 | H | H | H | H | H | H | H |
| 1-254 | P4 | NP11 | NP11 | H | H | H | H | H | H | H |
| 1-255 | P4 | NP12 | NP12 | H | H | H | H | H | H | H |
| 1-256 | P4 | NP13 | NP13 | H | H | H | H | H | H | H |
| 1-257 | P4 | NP14 | NP14 | H | H | H | H | H | H | H |
| 1-258 | P4 | NP15 | NP15 | H | H | H | H | H | H | H |
| 1-259 | P4 | NP16 | NP16 | H | H | H | H | H | H | H |
| 1-260 | P4 | NP17 | NP17 | H | H | H | H | H | H | H |
| 1-261 | P4 | NP18 | NP18 | H | H | H | H | H | H | H |
| 1-262 | P4 | PN1 | PN1 | H | H | H | H | H | H | H |
| 1-263 | P4 | FL1 | FL1 | H | H | H | H | H | H | H |
| 1-264 | P4 | TH1 | TH1 | H | H | H | H | H | H | H |
| 1-265 | P4 | TH2 | TH2 | H | H | H | H | H | H | H |
| 1-266 | P4 | TH3 | TH3 | H | H | H | H | H | H | H |
| 1-267 | P4 | BT1 | BT1 | H | H | H | H | H | H | H |
| 1-268 | P4 | BT3 | BT3 | H | H | H | H | H | H | H |
| 1-269 | P4 | BT6 | BT6 | H | H | H | H | H | H | H |
| 1-270 | P4 | BT7 | BT7 | H | H | H | H | H | H | H |
| 1-271 | P4 | BT9 | BT9 | H | H | H | H | H | H | H |
| 1-272 | P4 | CZ1 | CZ1 | H | H | H | H | H | H | H |
| 1-273 | P4 | CZ2 | CZ2 | H | H | H | H | H | H | H |
| 1-274 | P4 | CZ6 | CZ6 | H | H | H | H | H | H | H |
| 1-275 | BP1 | P1 | P1 | H | H | H | H | H | H | H |
| 1-276 | BP1 | P4 | P4 | H | H | H | H | H | H | H |

Table 2-7

| | | | | | | | | | | |
|---|---|---|---|---|---|---|---|---|---|---|
| 1-277 | BP1 | BP1 | BP1 | H | H | H | H | H | H | H |
| 1-278 | BP1 | BP2 | BP2 | H | H | H | H | H | H | H |
| 1-279 | BP1 | BP3 | BP3 | H | H | H | H | H | H | H |
| 1-280 | BP1 | BP4 | BP4 | H | H | H | H | H | H | H |
| 1-281 | BP1 | BP7 | BP7 | H | H | H | H | H | H | H |
| 1-282 | BP1 | TP1 | TP1 | H | H | H | H | H | H | H |
| 1-283 | BP1 | TP4 | TP4 | H | H | H | H | H | H | H |
| 1-284 | BP1 | TP5 | TP5 | H | H | H | H | H | H | H |
| 1-285 | BP1 | TP9 | TP9 | H | H | H | H | H | H | H |
| 1-286 | BP1 | TP13 | TP13 | H | H | H | H | H | H | H |
| 1-287 | BP1 | TP14 | TP14 | H | H | H | H | H | H | H |
| 1-288 | BP1 | TP15 | TP15 | H | H | H | H | H | H | H |
| 1-289 | BP1 | TP16 | TP16 | H | H | H | H | H | H | H |
| 1-290 | BP1 | TP17 | TP17 | H | H | H | H | H | H | H |
| 1-291 | BP1 | TP18 | TP18 | H | H | H | H | H | H | H |
| 1-292 | BP1 | TP19 | TP19 | H | H | H | H | H | H | H |
| 1-293 | BP1 | QP38 | QP38 | H | H | H | H | H | H | H |
| 1-294 | BP1 | QP64 | QP64 | H | H | H | H | H | H | H |
| 1-295 | BP1 | NP1 | NP1 | H | H | H | H | H | H | H |
| 1-296 | BP1 | NP2 | NP2 | H | H | H | H | H | H | H |
| 1-297 | BP1 | NP3 | NP3 | H | H | H | H | H | H | H |
| 1-298 | BP1 | NP4 | NP4 | H | H | H | H | H | H | H |
| 1-299 | BP1 | NP5 | NP5 | H | H | H | H | H | H | H |
| 1-300 | BP1 | NP6 | NP6 | H | H | H | H | H | H | H |
| 1-301 | BP1 | NP7 | NP7 | H | H | H | H | H | H | H |
| 1-302 | BP1 | NP8 | NP8 | H | H | H | H | H | H | H |
| 1-303 | BP1 | NP11 | NP11 | H | H | H | H | H | H | H |
| 1-304 | BP1 | NP13 | NP13 | H | H | H | H | H | H | H |
| 1-305 | BP1 | NP14 | NP14 | H | H | H | H | H | H | H |
| 1-306 | BP1 | NP15 | NP15 | H | H | H | H | H | H | H |
| 1-307 | BP1 | NP16 | NP16 | H | H | H | H | H | H | H |
| 1-308 | BP1 | NP17 | NP17 | H | H | H | H | H | H | H |
| 1-309 | BP1 | NP18 | NP18 | H | H | H | H | H | H | H |
| 1-310 | BP1 | PN1 | PN1 | H | H | H | H | H | H | H |
| 1-311 | BP1 | FL1 | FL1 | H | H | H | H | H | H | H |
| 1-312 | BP1 | TH2 | TH2 | H | H | H | H | H | H | H |
| 1-313 | BP1 | TH3 | TH3 | H | H | H | H | H | H | H |
| 1-314 | BP1 | BT1 | BT1 | H | H | H | H | H | H | H |
| 1-315 | BP1 | BT3 | BT3 | H | H | H | H | H | H | H |
| 1-316 | BP1 | BT6 | BT6 | H | H | H | H | H | H | H |
| 1-317 | BP1 | BT7 | BT7 | H | H | H | H | H | H | H |
| 1-318 | BP1 | BT9 | BT9 | H | H | H | H | H | H | H |
| 1-319 | BP1 | CZ1 | CZ1 | H | H | H | H | H | H | H |
| 1-320 | BP1 | CZ2 | CZ2 | H | H | H | H | H | H | H |
| 1-321 | BP1 | CZ6 | CZ6 | H | H | H | H | H | H | H |
| 1-322 | BP2 | P1 | P1 | H | H | H | H | H | H | H |

Table 2-8

| | | | | | | | | | | |
|---|---|---|---|---|---|---|---|---|---|---|
| 1-323 | BP2 | P2 | P2 | H | H | H | H | H | H | H |
| 1-324 | BP2 | P4 | P4 | H | H | H | H | H | H | H |
| 1-325 | BP2 | P5 | P5 | H | H | H | H | H | H | H |
| 1-326 | BP2 | BP1 | BP1 | H | H | H | H | H | H | H |
| 1-327 | BP2 | BP2 | BP2 | H | H | H | H | H | H | H |
| 1-328 | BP2 | BP3 | BP3 | H | H | H | H | H | H | H |
| 1-329 | BP2 | BP4 | BP4 | H | H | H | H | H | H | H |
| 1-330 | BP2 | BP7 | BP7 | H | H | H | H | H | H | H |
| 1-331 | BP2 | TP1 | TP1 | H | H | H | H | H | H | H |
| 1-332 | BP2 | TP4 | TP4 | H | H | H | H | H | H | H |
| 1-333 | BP2 | TP12 | TP12 | H | H | H | H | H | H | H |
| 1-334 | BP2 | TP15 | TP15 | H | H | H | H | H | H | H |
| 1-335 | BP2 | TP16 | TP16 | H | H | H | H | H | H | H |
| 1-336 | BP2 | TP17 | TP17 | H | H | H | H | H | H | H |
| 1-337 | BP2 | TP18 | TP18 | H | H | H | H | H | H | H |
| 1-338 | BP2 | QP38 | QP38 | H | H | H | H | H | H | H |
| 1-339 | BP2 | QP39 | QP39 | H | H | H | H | H | H | H |
| 1-340 | BP2 | QP44 | QP44 | H | H | H | H | H | H | H |
| 1-341 | BP2 | QP45 | QP45 | H | H | H | H | H | H | H |
| 1-342 | BP2 | QP63 | QP63 | H | H | H | H | H | H | H |
| 1-343 | BP2 | QP64 | QP64 | H | H | H | H | H | H | H |
| 1-344 | BP2 | NP1 | NP1 | H | H | H | H | H | H | H |
| 1-345 | BP2 | NP3 | NP3 | H | H | H | H | H | H | H |
| 1-346 | BP2 | NP4 | NP4 | H | H | H | H | H | H | H |
| 1-347 | BP2 | NP5 | NP5 | H | H | H | H | H | H | H |
| 1-348 | BP2 | NP6 | NP6 | H | H | H | H | H | H | H |
| 1-349 | BP2 | NP7 | NP7 | H | H | H | H | H | H | H |
| 1-350 | BP2 | NP8 | NP8 | H | H | H | H | H | H | H |
| 1-351 | BP2 | NP11 | NP11 | H | H | H | H | H | H | H |
| 1-352 | BP2 | NP13 | NP13 | H | H | H | H | H | H | H |
| 1-353 | BP2 | NP14 | NP14 | H | H | H | H | H | H | H |
| 1-354 | BP2 | NP15 | NP15 | H | H | H | H | H | H | H |
| 1-355 | BP2 | NP16 | NP16 | H | H | H | H | H | H | H |
| 1-356 | BP2 | NP17 | NP17 | H | H | H | H | H | H | H |
| 1-357 | BP2 | NP18 | NP18 | H | H | H | H | H | H | H |
| 1-358 | BP2 | PN1 | PN1 | H | H | H | H | H | H | H |
| 1-359 | BP2 | FL1 | FL1 | H | H | H | H | H | H | H |
| 1-360 | BP2 | TH3 | TH3 | H | H | H | H | H | H | H |
| 1-361 | BP2 | BT1 | BT1 | H | H | H | H | H | H | H |
| 1-362 | BP2 | BT3 | BT3 | H | H | H | H | H | H | H |
| 1-363 | BP2 | BT6 | BT6 | H | H | H | H | H | H | H |
| 1-364 | BP2 | BT7 | BT7 | H | H | H | H | H | H | H |
| 1-365 | BP2 | CZ1 | CZ1 | H | H | H | H | H | H | H |
| 1-366 | BP2 | CZ6 | CZ6 | H | H | H | H | H | H | H |
| 1-367 | BP3 | P2 | P2 | H | H | H | H | H | H | H |
| 1-368 | BP3 | P4 | P4 | H | H | H | H | H | H | H |

Table 2-9

| | | | | | | | | | | |
|---|---|---|---|---|---|---|---|---|---|---|
| 1-369 | BP3 | P5 | P5 | H | H | H | H | H | H | H |
| 1-370 | BP3 | P8 | P8 | H | H | H | H | H | H | H |
| 1-371 | BP3 | BP1 | BP1 | H | H | H | H | H | H | H |
| 1-372 | BP3 | BP2 | BP2 | H | H | H | H | H | H | H |
| 1-373 | BP3 | BP3 | BP3 | H | H | H | H | H | H | H |
| 1-374 | BP3 | BP4 | BP4 | H | H | H | H | H | H | H |
| 1-375 | BP3 | BP7 | BP7 | H | H | H | H | H | H | H |
| 1-376 | BP3 | TP1 | TP1 | H | H | H | H | H | H | H |
| 1-377 | BP3 | TP4 | TP4 | H | H | H | H | H | H | H |
| 1-378 | BP3 | TP12 | TP12 | H | H | H | H | H | H | H |
| 1-379 | BP3 | TP15 | TP15 | H | H | H | H | H | H | H |
| 1-380 | BP3 | TP16 | TP16 | H | H | H | H | H | H | H |
| 1-381 | BP3 | TP17 | TP17 | H | H | H | H | H | H | H |
| 1-382 | BP3 | TP18 | TP18 | H | H | H | H | H | H | H |
| 1-383 | BP3 | QP38 | QP38 | H | H | H | H | H | H | H |
| 1-384 | BP3 | QP39 | QP39 | H | H | H | H | H | H | H |
| 1-385 | BP3 | QP44 | QP44 | H | H | H | H | H | H | H |
| 1-386 | BP3 | QP45 | QP45 | H | H | H | H | H | H | H |
| 1-387 | BP3 | QP63 | QP63 | H | H | H | H | H | H | H |
| 1-388 | BP3 | QP64 | QP64 | H | H | H | H | H | H | H |
| 1-389 | BP3 | NP1 | NP1 | H | H | H | H | H | H | H |
| 1-390 | BP3 | NP3 | NP3 | H | H | H | H | H | H | H |
| 1-391 | BP3 | NP4 | NP4 | H | H | H | H | H | H | H |
| 1-392 | BP3 | NP5 | NP5 | H | H | H | H | H | H | H |
| 1-393 | BP3 | NP6 | NP6 | H | H | H | H | H | H | H |
| 1-394 | BP3 | NP7 | NP7 | H | H | H | H | H | H | H |
| 1-395 | BP3 | NP8 | NP8 | H | H | H | H | H | H | H |
| 1-396 | BP3 | NP11 | NP11 | H | H | H | H | H | H | H |
| 1-397 | BP3 | NP13 | NP13 | H | H | H | H | H | H | H |
| 1-398 | BP3 | NP14 | NP14 | H | H | H | H | H | H | H |
| 1-399 | BP3 | NP15 | NP15 | H | H | H | H | H | H | H |
| 1-400 | BP3 | NP16 | NP16 | H | H | H | H | H | H | H |
| 1-401 | BP3 | NP17 | NP17 | H | H | H | H | H | H | H |
| 1-402 | BP3 | NP18 | NP18 | H | H | H | H | H | H | H |
| 1-403 | BP3 | PN1 | PN1 | H | H | H | H | H | H | H |
| 1-404 | BP3 | FL1 | FL1 | H | H | H | H | H | H | H |
| 1-405 | BP3 | TH3 | TH3 | H | H | H | H | H | H | H |
| 1-406 | BP3 | BT1 | BT1 | H | H | H | H | H | H | H |
| 1-407 | BP3 | BT3 | BT3 | H | H | H | H | H | H | H |
| 1-408 | BP3 | BT6 | BT6 | H | H | H | H | H | H | H |
| 1-409 | BP3 | BT7 | BT7 | H | H | H | H | H | H | H |
| 1-410 | BP3 | CZ1 | CZ1 | H | H | H | H | H | H | H |
| 1-411 | BP3 | CZ6 | CZ6 | H | H | H | H | H | H | H |
| 1-412 | TP1 | P1 | P1 | H | H | H | H | H | H | H |
| 1-413 | TP1 | P2 | P2 | H | H | H | H | H | H | H |
| 1-414 | TP1 | P4 | P4 | H | H | H | H | H | H | H |

Table 2-10

| | | | | | | | | | | |
|---|---|---|---|---|---|---|---|---|---|---|
| 1-415 | TP1 | P5 | P5 | H | H | H | H | H | H | H |
| 1-416 | TP1 | P8 | P8 | H | H | H | H | H | H | H |
| 1-417 | TP1 | BP1 | BP1 | H | H | H | H | H | H | H |
| 1-418 | TP1 | BP2 | BP2 | H | H | H | H | H | H | H |
| 1-419 | TP1 | BP3 | BP3 | H | H | H | H | H | H | H |
| 1-420 | TP1 | BP4 | BP4 | H | H | H | H | H | H | H |
| 1-421 | TP1 | BP7 | BP7 | H | H | H | H | H | H | H |
| 1-422 | TP1 | TP1 | TP1 | H | H | H | H | H | H | H |
| 1-423 | TP1 | TP4 | TP4 | H | H | H | H | H | H | H |
| 1-424 | TP1 | TP12 | TP12 | H | H | H | H | H | H | H |
| 1-425 | TP1 | TP15 | TP15 | H | H | H | H | H | H | H |
| 1-426 | TP1 | TP16 | TP16 | H | H | H | H | H | H | H |
| 1-427 | TP1 | TP17 | TP17 | H | H | H | H | H | H | H |
| 1-428 | TP1 | TP18 | TP18 | H | H | H | H | H | H | H |
| 1-429 | TP1 | QP38 | QP38 | H | H | H | H | H | H | H |
| 1-430 | TP1 | QP39 | QP39 | H | H | H | H | H | H | H |
| 1-431 | TP1 | QP44 | QP44 | H | H | H | H | H | H | H |
| 1-432 | TP1 | QP45 | QP45 | H | H | H | H | H | H | H |
| 1-433 | TP1 | QP63 | QP63 | H | H | H | H | H | H | H |
| 1-434 | TP1 | QP64 | QP64 | H | H | H | H | H | H | H |
| 1-435 | TP1 | NP1 | NP1 | H | H | H | H | H | H | H |
| 1-436 | TP1 | NP3 | NP3 | H | H | H | H | H | H | H |
| 1-437 | TP1 | NP4 | NP4 | H | H | H | H | H | H | H |
| 1-438 | TP1 | NP5 | NP5 | H | H | H | H | H | H | H |
| 1-439 | TP1 | NP6 | NP6 | H | H | H | H | H | H | H |
| 1-440 | TP1 | NP7 | NP7 | H | H | H | H | H | H | H |
| 1-441 | TP1 | NP8 | NP8 | H | H | H | H | H | H | H |
| 1-442 | TP1 | NP11 | NP11 | H | H | H | H | H | H | H |
| 1-443 | TP1 | NP13 | NP13 | H | H | H | H | H | H | H |
| 1-444 | TP1 | NP14 | NP14 | H | H | H | H | H | H | H |
| 1-445 | TP1 | NP15 | NP15 | H | H | H | H | H | H | H |
| 1-446 | TP1 | NP16 | NP16 | H | H | H | H | H | H | H |
| 1-447 | TP1 | NP17 | NP17 | H | H | H | H | H | H | H |
| 1-448 | TP1 | NP18 | NP18 | H | H | H | H | H | H | H |
| 1-449 | TP1 | PN1 | PN1 | H | H | H | H | H | H | H |
| 1-450 | TP1 | FL1 | FL1 | H | H | H | H | H | H | H |
| 1-451 | TP1 | TH3 | TH3 | H | H | H | H | H | H | H |
| 1-452 | TP1 | BT1 | BT1 | H | H | H | H | H | H | H |
| 1-453 | TP1 | BT3 | BT3 | H | H | H | H | H | H | H |
| 1-454 | TP1 | BT6 | BT6 | H | H | H | H | H | H | H |
| 1-455 | TP1 | BT7 | BT7 | H | H | H | H | H | H | H |
| 1-456 | TP1 | CZ1 | CZ1 | H | H | H | H | H | H | H |
| 1-457 | TP1 | CZ6 | CZ6 | H | H | H | H | H | H | H |
| 1-458 | TP16 | P1 | P1 | H | H | H | H | H | H | H |
| 1-459 | TP16 | P2 | P2 | H | H | H | H | H | H | H |
| 1-460 | TP16 | P4 | P4 | H | H | H | H | H | H | H |

Table 2-11

| | | | | | | | | | | |
|---|---|---|---|---|---|---|---|---|---|---|
| 1-461 | TP16 | P5 | P5 | H | H | H | H | H | H | H |
| 1-462 | TP16 | P8 | P8 | H | H | H | H | H | H | H |
| 1-463 | TP16 | BP1 | BP1 | H | H | H | H | H | H | H |
| 1-464 | TP16 | BP2 | BP2 | H | H | H | H | H | H | H |
| 1-465 | TP16 | BP3 | BP3 | H | H | H | H | H | H | H |
| 1-466 | TP16 | BP4 | BP4 | H | H | H | H | H | H | H |
| 1-467 | TP16 | BP7 | BP7 | H | H | H | H | H | H | H |
| 1-468 | TP16 | TP1 | TP1 | H | H | H | H | H | H | H |
| 1-469 | TP16 | TP4 | TP4 | H | H | H | H | H | H | H |
| 1-470 | TP16 | TP12 | TP12 | H | H | H | H | H | H | H |
| 1-471 | TP16 | TP15 | TP15 | H | H | H | H | H | H | H |
| 1-472 | TP16 | TP16 | TP16 | H | H | H | H | H | H | H |
| 1-473 | TP16 | TP17 | TP17 | H | H | H | H | H | H | H |
| 1-474 | TP16 | TP18 | TP18 | H | H | H | H | H | H | H |
| 1-475 | TP16 | QP38 | QP38 | H | H | H | H | H | H | H |
| 1-476 | TP16 | QP39 | QP39 | H | H | H | H | H | H | H |
| 1-477 | TP16 | QP44 | QP44 | H | H | H | H | H | H | H |
| 1-478 | TP16 | QP45 | QP45 | H | H | H | H | H | H | H |
| 1-479 | TP16 | QP63 | QP63 | H | H | H | H | H | H | H |
| 1-480 | TP16 | QP64 | QP64 | H | H | H | H | H | H | H |
| 1-481 | TP16 | NP1 | NP1 | H | H | H | H | H | H | H |
| 1-482 | TP16 | NP3 | NP3 | H | H | H | H | H | H | H |
| 1-483 | TP16 | NP4 | NP4 | H | H | H | H | H | H | H |
| 1-484 | TP16 | NP5 | NP5 | H | H | H | H | H | H | H |
| 1-485 | TP16 | NP6 | NP6 | H | H | H | H | H | H | H |
| 1-486 | TP16 | NP7 | NP7 | H | H | H | H | H | H | H |
| 1-487 | TP16 | NP8 | NP8 | H | H | H | H | H | H | H |
| 1-488 | TP16 | NP11 | NP11 | H | H | H | H | H | H | H |
| 1-489 | TP16 | NP13 | NP13 | H | H | H | H | H | H | H |
| 1-490 | TP16 | NP14 | NP14 | H | H | H | H | H | H | H |
| 1-491 | TP16 | NP15 | NP15 | H | H | H | H | H | H | H |
| 1-492 | TP16 | NP16 | NP16 | H | H | H | H | H | H | H |
| 1-493 | TP16 | NP17 | NP17 | H | H | H | H | H | H | H |
| 1-494 | TP16 | NP18 | NP18 | H | H | H | H | H | H | H |
| 1-495 | TP16 | PN1 | PN1 | H | H | H | H | H | H | H |
| 1-496 | TP16 | FL1 | FL1 | H | H | H | H | H | H | H |
| 1-497 | TP16 | TH3 | TH3 | H | H | H | H | H | H | H |
| 1-498 | TP16 | BT1 | BT1 | H | H | H | H | H | H | H |
| 1-499 | TP16 | BT3 | BT3 | H | H | H | H | H | H | H |
| 1-500 | TP16 | BT6 | BT6 | H | H | H | H | H | H | H |
| 1-501 | TP16 | BT7 | BT7 | H | H | H | H | H | H | H |
| 1-502 | TP16 | CZ1 | CZ1 | H | H | H | H | H | H | H |
| 1-503 | TP16 | CZ6 | CZ6 | H | H | H | H | H | H | H |
| 1-504 | QP38 | P1 | P1 | H | H | H | H | H | H | H |
| 1-505 | QP38 | P2 | P2 | H | H | H | H | H | H | H |
| 1-506 | QP38 | P4 | P4 | H | H | H | H | H | H | H |

Table 2-12

| | | | | | | | | | | |
|---|---|---|---|---|---|---|---|---|---|---|
| 1-507 | QP38 | P5 | P5 | H | H | H | H | H | H | H |
| 1-508 | QP38 | P8 | P8 | H | H | H | H | H | H | H |
| 1-509 | QP38 | BP1 | BP1 | H | H | H | H | H | H | H |
| 1-510 | QP38 | BP2 | BP2 | H | H | H | H | H | H | H |
| 1-511 | QP38 | BP3 | BP3 | H | H | H | H | H | H | H |
| 1-512 | QP38 | BP4 | BP4 | H | H | H | H | H | H | H |
| 1-513 | QP38 | BP7 | BP7 | H | H | H | H | H | H | H |
| 1-514 | QP38 | TP1 | TP1 | H | H | H | H | H | H | H |
| 1-515 | QP38 | TP4 | TP4 | H | H | H | H | H | H | H |
| 1-516 | QP38 | TP12 | TP12 | H | H | H | H | H | H | H |
| 1-517 | QP38 | TP15 | TP15 | H | H | H | H | H | H | H |
| 1-518 | QP38 | TP16 | TP16 | H | H | H | H | H | H | H |
| 1-519 | QP38 | TP17 | TP17 | H | H | H | H | H | H | H |
| 1-520 | QP38 | TP18 | TP18 | H | H | H | H | H | H | H |
| 1-521 | QP38 | QP38 | QP38 | H | H | H | H | H | H | H |
| 1-522 | QP38 | QP39 | QP39 | H | H | H | H | H | H | H |
| 1-523 | QP38 | QP44 | QP44 | H | H | H | H | H | H | H |
| 1-524 | QP38 | QP45 | QP45 | H | H | H | H | H | H | H |
| 1-525 | QP38 | QP63 | QP63 | H | H | H | H | H | H | H |
| 1-526 | QP38 | QP64 | QP64 | H | H | H | H | H | H | H |
| 1-527 | QP38 | NP1 | NP1 | H | H | H | H | H | H | H |
| 1-528 | QP38 | NP3 | NP3 | H | H | H | H | H | H | H |
| 1-529 | QP38 | NP4 | NP4 | H | H | H | H | H | H | H |
| 1-530 | QP38 | NP5 | NP5 | H | H | H | H | H | H | H |
| 1-531 | QP38 | NP6 | NP6 | H | H | H | H | H | H | H |
| 1-532 | QP38 | NP7 | NP7 | H | H | H | H | H | H | H |
| 1-533 | QP38 | NP8 | NP8 | H | H | H | H | H | H | H |
| 1-534 | QP38 | NP11 | NP11 | H | H | H | H | H | H | H |
| 1-535 | QP38 | NP13 | NP13 | H | H | H | H | H | H | H |
| 1-536 | QP38 | NP14 | NP14 | H | H | H | H | H | H | H |
| 1-537 | QP38 | NP15 | NP15 | H | H | H | H | H | H | H |
| 1-538 | QP38 | NP16 | NP16 | H | H | H | H | H | H | H |
| 1-539 | QP38 | NP17 | NP17 | H | H | H | H | H | H | H |
| 1-540 | QP38 | NP18 | NP18 | H | H | H | H | H | H | H |
| 1-541 | QP38 | PN1 | PN1 | H | H | H | H | H | H | H |
| 1-542 | QP38 | FL1 | FL1 | H | H | H | H | H | H | H |
| 1-543 | QP38 | TH3 | TH3 | H | H | H | H | H | H | H |
| 1-544 | QP38 | BT1 | BT1 | H | H | H | H | H | H | H |
| 1-545 | QP38 | BT3 | BT3 | H | H | H | H | H | H | H |
| 1-546 | QP38 | BT6 | BT6 | H | H | H | H | H | H | H |
| 1-547 | QP38 | BT7 | BT7 | H | H | H | H | H | H | H |
| 1-548 | QP38 | CZ1 | CZ1 | H | H | H | H | H | H | H |
| 1-549 | QP38 | CZ6 | CZ6 | H | H | H | H | H | H | H |
| 1-550 | QP45 | P1 | P1 | H | H | H | H | H | H | H |
| 1-551 | QP45 | P2 | P2 | H | H | H | H | H | H | H |
| 1-552 | QP45 | P4 | P4 | H | H | H | H | H | H | H |

Table 2-13

| | | | | | | | | | | |
|---|---|---|---|---|---|---|---|---|---|---|
| 1-553 | QP45 | P5 | P5 | H | H | H | H | H | H | H |
| 1-554 | QP45 | P8 | P8 | H | H | H | H | H | H | H |
| 1-555 | QP45 | BP1 | BP1 | H | H | H | H | H | H | H |
| 1-556 | QP45 | BP2 | BP2 | H | H | H | H | H | H | H |
| 1-557 | QP45 | BP3 | BP3 | H | H | H | H | H | H | H |
| 1-558 | QP45 | BP4 | BP4 | H | H | H | H | H | H | H |
| 1-559 | QP45 | BP7 | BP7 | H | H | H | H | H | H | H |
| 1-560 | QP45 | TP1 | TP1 | H | H | H | H | H | H | H |
| 1-561 | QP45 | TP4 | TP4 | H | H | H | H | H | H | H |
| 1-562 | QP45 | TP12 | TP12 | H | H | H | H | H | H | H |
| 1-563 | QP45 | TP15 | TP15 | H | H | H | H | H | H | H |
| 1-564 | QP45 | TP16 | TP16 | H | H | H | H | H | H | H |
| 1-565 | QP45 | TP17 | TP17 | H | H | H | H | H | H | H |
| 1-566 | QP45 | TP18 | TP18 | H | H | H | H | H | H | H |
| 1-567 | QP45 | QP38 | QP38 | H | H | H | H | H | H | H |
| 1-568 | QP45 | QP39 | QP39 | H | H | H | H | H | H | H |
| 1-569 | QP45 | QP44 | QP44 | H | H | H | H | H | H | H |
| 1-570 | QP45 | QP45 | QP45 | H | H | H | H | H | H | H |
| 1-571 | QP45 | QP63 | QP63 | H | H | H | H | H | H | H |
| 1-572 | QP45 | QP64 | QP64 | H | H | H | H | H | H | H |
| 1-573 | QP45 | NP1 | NP1 | H | H | H | H | H | H | H |
| 1-574 | QP45 | NP3 | NP3 | H | H | H | H | H | H | H |
| 1-575 | QP45 | NP4 | NP4 | H | H | H | H | H | H | H |
| 1-576 | QP45 | NP5 | NP5 | H | H | H | H | H | H | H |
| 1-577 | QP45 | NP6 | NP6 | H | H | H | H | H | H | H |
| 1-578 | QP45 | NP7 | NP7 | H | H | H | H | H | H | H |
| 1-579 | QP45 | NP8 | NP8 | H | H | H | H | H | H | H |
| 1-580 | QP45 | NP11 | NP11 | H | H | H | H | H | H | H |
| 1-581 | QP45 | NP13 | NP13 | H | H | H | H | H | H | H |
| 1-582 | QP45 | NP14 | NP14 | H | H | H | H | H | H | H |
| 1-583 | QP45 | NP15 | NP15 | H | H | H | H | H | H | H |
| 1-584 | QP45 | NP16 | NP16 | H | H | H | H | H | H | H |
| 1-585 | QP45 | NP17 | NP17 | H | H | H | H | H | H | H |
| 1-586 | QP45 | NP18 | NP18 | H | H | H | H | H | H | H |
| 1-587 | QP45 | PN1 | PN1 | H | H | H | H | H | H | H |
| 1-588 | QP45 | FL1 | FL1 | H | H | H | H | H | H | H |
| 1-589 | QP45 | TH3 | TH3 | H | H | H | H | H | H | H |
| 1-590 | QP45 | BT1 | BT1 | H | H | H | H | H | H | H |
| 1-591 | QP45 | BT3 | BT3 | H | H | H | H | H | H | H |
| 1-592 | QP45 | BT6 | BT6 | H | H | H | H | H | H | H |
| 1-593 | QP45 | BT7 | BT7 | H | H | H | H | H | H | H |
| 1-594 | QP45 | CZ1 | CZ1 | H | H | H | H | H | H | H |
| 1-595 | QP45 | CZ6 | CZ6 | H | H | H | H | H | H | H |
| 1-596 | NP11 | P1 | P1 | H | H | H | H | H | H | H |
| 1-597 | NP11 | P2 | P2 | H | H | H | H | H | H | H |
| 1-598 | NP11 | P4 | P4 | H | H | H | H | H | H | H |

Table 2-14

| | | | | | | | | | | |
|---|---|---|---|---|---|---|---|---|---|---|
| 1-599 | NP11 | P5 | P5 | H | H | H | H | H | H | H |
| 1-600 | NP11 | P8 | P8 | H | H | H | H | H | H | H |
| 1-601 | NP11 | BP1 | BP1 | H | H | H | H | H | H | H |
| 1-602 | NP11 | BP2 | BP2 | H | H | H | H | H | H | H |
| 1-603 | NP11 | BP3 | BP3 | H | H | H | H | H | H | H |
| 1-604 | NP11 | BP4 | BP4 | H | H | H | H | H | H | H |
| 1-605 | NP11 | BP7 | BP7 | H | H | H | H | H | H | H |
| 1-606 | NP11 | TP1 | TP1 | H | H | H | H | H | H | H |
| 1-607 | NP11 | TP4 | TP4 | H | H | H | H | H | H | H |
| 1-608 | NP11 | TP12 | TP12 | H | H | H | H | H | H | H |
| 1-609 | NP11 | TP15 | TP15 | H | H | H | H | H | H | H |
| 1-610 | NP11 | TP16 | TP16 | H | H | H | H | H | H | H |
| 1-611 | NP11 | TP17 | TP17 | H | H | H | H | H | H | H |
| 1-612 | NP11 | TP18 | TP18 | H | H | H | H | H | H | H |
| 1-613 | NP11 | QP38 | QP38 | H | H | H | H | H | H | H |
| 1-614 | NP11 | QP39 | QP39 | H | H | H | H | H | H | H |
| 1-615 | NP11 | QP44 | QP44 | H | H | H | H | H | H | H |
| 1-616 | NP11 | QP45 | QP45 | H | H | H | H | H | H | H |
| 1-617 | NP11 | QP63 | QP63 | H | H | H | H | H | H | H |
| 1-618 | NP11 | QP64 | QP64 | H | H | H | H | H | H | H |
| 1-619 | NP11 | NP1 | NP1 | H | H | H | H | H | H | H |
| 1-620 | NP11 | NP3 | NP3 | H | H | H | H | H | H | H |
| 1-621 | NP11 | NP4 | NP4 | H | H | H | H | H | H | H |
| 1-622 | NP11 | NP5 | NP5 | H | H | H | H | H | H | H |
| 1-623 | NP11 | NP6 | NP6 | H | H | H | H | H | H | H |
| 1-624 | NP11 | NP7 | NP7 | H | H | H | H | H | H | H |
| 1-625 | NP11 | NP8 | NP8 | H | H | H | H | H | H | H |
| 1-626 | NP11 | NP11 | NP11 | H | H | H | H | H | H | H |
| 1-627 | NP11 | NP13 | NP13 | H | H | H | H | H | H | H |
| 1-628 | NP11 | NP14 | NP14 | H | H | H | H | H | H | H |
| 1-629 | NP11 | NP15 | NP15 | H | H | H | H | H | H | H |
| 1-630 | NP11 | NP16 | NP16 | H | H | H | H | H | H | H |
| 1-631 | NP11 | NP17 | NP17 | H | H | H | H | H | H | H |
| 1-632 | NP11 | NP18 | NP18 | H | H | H | H | H | H | H |
| 1-633 | NP11 | PN1 | PN1 | H | H | H | H | H | H | H |
| 1-634 | NP11 | FL1 | FL1 | H | H | H | H | H | H | H |
| 1-635 | NP11 | TH3 | TH3 | H | H | H | H | H | H | H |
| 1-636 | NP11 | BT1 | BT1 | H | H | H | H | H | H | H |
| 1-637 | NP11 | BT3 | BT3 | H | H | H | H | H | H | H |
| 1-638 | NP11 | BT6 | BT6 | H | H | H | H | H | H | H |
| 1-639 | NP11 | BT7 | BT7 | H | H | H | H | H | H | H |
| 1-640 | NP11 | CZ1 | CZ1 | H | H | H | H | H | H | H |
| 1-641 | NP11 | CZ6 | CZ6 | H | H | H | H | H | H | H |
| 1-642 | NP14 | P1 | P1 | H | H | H | H | H | H | H |
| 1-643 | NP14 | P2 | P2 | H | H | H | H | H | H | H |
| 1-644 | NP14 | P4 | P4 | H | H | H | H | H | H | H |

Table 2-15

| | | | | | | | | | | |
|---|---|---|---|---|---|---|---|---|---|---|
| 1-645 | NP14 | P5 | P5 | H | H | H | H | H | H | H |
| 1-646 | NP14 | P8 | P8 | H | H | H | H | H | H | H |
| 1-647 | NP14 | BP1 | BP1 | H | H | H | H | H | H | H |
| 1-648 | NP14 | BP2 | BP2 | H | H | H | H | H | H | H |
| 1-649 | NP14 | BP3 | BP3 | H | H | H | H | H | H | H |
| 1-650 | NP14 | BP4 | BP4 | H | H | H | H | H | H | H |
| 1-651 | NP14 | BP7 | BP7 | H | H | H | H | H | H | H |
| 1-652 | NP14 | TP1 | TP1 | H | H | H | H | H | H | H |
| 1-653 | NP14 | TP4 | TP4 | H | H | H | H | H | H | H |
| 1-654 | NP14 | TP12 | TP12 | H | H | H | H | H | H | H |
| 1-655 | NP14 | TP15 | TP15 | H | H | H | H | H | H | H |
| 1-656 | NP14 | TP16 | TP16 | H | H | H | H | H | H | H |
| 1-657 | NP14 | TP17 | TP17 | H | H | H | H | H | H | H |
| 1-658 | NP14 | TP18 | TP18 | H | H | H | H | H | H | H |
| 1-659 | NP14 | QP38 | QP38 | H | H | H | H | H | H | H |
| 1-660 | NP14 | QP39 | QP39 | H | H | H | H | H | H | H |
| 1-661 | NP14 | QP44 | QP44 | H | H | H | H | H | H | H |
| 1-662 | NP14 | QP45 | QP45 | H | H | H | H | H | H | H |
| 1-663 | NP14 | QP63 | QP63 | H | H | H | H | H | H | H |
| 1-664 | NP14 | QP64 | QP64 | H | H | H | H | H | H | H |
| 1-665 | NP14 | NP1 | NP1 | H | H | H | H | H | H | H |
| 1-666 | NP14 | NP3 | NP3 | H | H | H | H | H | H | H |
| 1-667 | NP14 | NP4 | NP4 | H | H | H | H | H | H | H |
| 1-668 | NP14 | NP5 | NP5 | H | H | H | H | H | H | H |
| 1-669 | NP14 | NP6 | NP6 | H | H | H | H | H | H | H |
| 1-670 | NP14 | NP7 | NP7 | H | H | H | H | H | H | H |
| 1-671 | NP14 | NP8 | NP8 | H | H | H | H | H | H | H |
| 1-672 | NP14 | NP11 | NP11 | H | H | H | H | H | H | H |
| 1-673 | NP14 | NP13 | NP13 | H | H | H | H | H | H | H |
| 1-674 | NP14 | NP14 | NP14 | H | H | H | H | H | H | H |
| 1-675 | NP14 | NP15 | NP15 | H | H | H | H | H | H | H |
| 1-676 | NP14 | NP16 | NP16 | H | H | H | H | H | H | H |
| 1-677 | NP14 | NP17 | NP17 | H | H | H | H | H | H | H |
| 1-678 | NP14 | NP18 | NP18 | H | H | H | H | H | H | H |
| 1-679 | NP14 | PN1 | PN1 | H | H | H | H | H | H | H |
| 1-680 | NP14 | FL1 | FL1 | H | H | H | H | H | H | H |
| 1-681 | NP14 | TH3 | TH3 | H | H | H | H | H | H | H |
| 1-682 | NP14 | BT1 | BT1 | H | H | H | H | H | H | H |
| 1-683 | NP14 | BT3 | BT3 | H | H | H | H | H | H | H |
| 1-684 | NP14 | BT6 | BT6 | H | H | H | H | H | H | H |
| 1-685 | NP14 | BT7 | BT7 | H | H | H | H | H | H | H |
| 1-686 | NP14 | CZ1 | CZ1 | H | H | H | H | H | H | H |
| 1-687 | NP14 | CZ6 | CZ6 | H | H | H | H | H | H | H |
| 1-688 | NP16 | P1 | P1 | H | H | H | H | H | H | H |
| 1-689 | NP16 | P2 | P2 | H | H | H | H | H | H | H |
| 1-690 | NP16 | P4 | P4 | H | H | H | H | H | H | H |

Table 2-16

| | | | | | | | | | | |
|---|---|---|---|---|---|---|---|---|---|---|
| 1-691 | NP16 | P5 | P5 | H | H | H | H | H | H | H |
| 1-692 | NP16 | P8 | P8 | H | H | H | H | H | H | H |
| 1-693 | NP16 | BP1 | BP1 | H | H | H | H | H | H | H |
| 1-694 | NP16 | BP2 | BP2 | H | H | H | H | H | H | H |
| 1-695 | NP16 | BP3 | BP3 | H | H | H | H | H | H | H |
| 1-696 | NP16 | BP4 | BP4 | H | H | H | H | H | H | H |
| 1-697 | NP16 | BP7 | BP7 | H | H | H | H | H | H | H |
| 1-698 | NP16 | TP1 | TP1 | H | H | H | H | H | H | H |
| 1-699 | NP16 | TP4 | TP4 | H | H | H | H | H | H | H |
| 1-700 | NP16 | TP12 | TP12 | H | H | H | H | H | H | H |
| 1-701 | NP16 | TP15 | TP15 | H | H | H | H | H | H | H |
| 1-702 | NP16 | TP16 | TP16 | H | H | H | H | H | H | H |
| 1-703 | NP16 | TP17 | TP17 | H | H | H | H | H | H | H |
| 1-704 | NP16 | TP18 | TP18 | H | H | H | H | H | H | H |
| 1-705 | NP16 | QP38 | QP38 | H | H | H | H | H | H | H |
| 1-706 | NP16 | QP39 | QP39 | H | H | H | H | H | H | H |
| 1-707 | NP16 | QP44 | QP44 | H | H | H | H | H | H | H |
| 1-708 | NP16 | QP45 | QP45 | H | H | H | H | H | H | H |
| 1-709 | NP16 | QP63 | QP63 | H | H | H | H | H | H | H |
| 1-710 | NP16 | QP64 | QP64 | H | H | H | H | H | H | H |
| 1-711 | NP16 | NP1 | NP1 | H | H | H | H | H | H | H |
| 1-712 | NP16 | NP3 | NP3 | H | H | H | H | H | H | H |
| 1-713 | NP16 | NP4 | NP4 | H | H | H | H | H | H | H |
| 1-714 | NP16 | NP5 | NP5 | H | H | H | H | H | H | H |
| 1-715 | NP16 | NP6 | NP6 | H | H | H | H | H | H | H |
| 1-716 | NP16 | NP7 | NP7 | H | H | H | H | H | H | H |
| 1-717 | NP16 | NP8 | NP8 | H | H | H | H | H | H | H |
| 1-718 | NP16 | NP11 | NP11 | H | H | H | H | H | H | H |
| 1-719 | NP16 | NP13 | NP13 | H | H | H | H | H | H | H |
| 1-720 | NP16 | NP14 | NP14 | H | H | H | H | H | H | H |
| 1-721 | NP16 | NP15 | NP15 | H | H | H | H | H | H | H |
| 1-722 | NP16 | NP16 | NP16 | H | H | H | H | H | H | H |
| 1-723 | NP16 | NP17 | NP17 | H | H | H | H | H | H | H |
| 1-724 | NP16 | NP18 | NP18 | H | H | H | H | H | H | H |
| 1-725 | NP16 | PN1 | PN1 | H | H | H | H | H | H | H |
| 1-726 | NP16 | FL1 | FL1 | H | H | H | H | H | H | H |
| 1-727 | NP16 | TH3 | TH3 | H | H | H | H | H | H | H |
| 1-728 | NP16 | BT1 | BT1 | H | H | H | H | H | H | H |
| 1-729 | NP16 | BT3 | BT3 | H | H | H | H | H | H | H |
| 1-730 | NP16 | BT6 | BT6 | H | H | H | H | H | H | H |
| 1-731 | NP16 | BT7 | BT7 | H | H | H | H | H | H | H |
| 1-732 | NP16 | CZ1 | CZ1 | H | H | H | H | H | H | H |
| 1-733 | NP16 | CZ6 | CZ6 | H | H | H | H | H | H | H |
| 1-734 | NP18 | P1 | P1 | H | H | H | H | H | H | H |
| 1-735 | NP18 | P2 | P2 | H | H | H | H | H | H | H |
| 1-736 | NP18 | P4 | P4 | H | H | H | H | H | H | H |

Table 2-17

| | | | | | | | | | | |
|---|---|---|---|---|---|---|---|---|---|---|
| 1-737 | NP18 | P5 | P5 | H | H | H | H | H | H | H |
| 1-738 | NP18 | P8 | P8 | H | H | H | H | H | H | H |
| 1-739 | NP18 | BP1 | BP1 | H | H | H | H | H | H | H |
| 1-740 | NP18 | BP2 | BP2 | H | H | H | H | H | H | H |
| 1-741 | NP18 | BP3 | BP3 | H | H | H | H | H | H | H |
| 1-742 | NP18 | BP4 | BP4 | H | H | H | H | H | H | H |
| 1-743 | NP18 | BP7 | BP7 | H | H | H | H | H | H | H |
| 1-744 | NP18 | TP1 | TP1 | H | H | H | H | H | H | H |
| 1-745 | NP18 | TP4 | TP4 | H | H | H | H | H | H | H |
| 1-746 | NP18 | TP12 | TP12 | H | H | H | H | H | H | H |
| 1-747 | NP18 | TP15 | TP15 | H | H | H | H | H | H | H |
| 1-748 | NP18 | TP16 | TP16 | H | H | H | H | H | H | H |
| 1-749 | NP18 | TP17 | TP17 | H | H | H | H | H | H | H |
| 1-750 | NP18 | TP18 | TP18 | H | H | H | H | H | H | H |
| 1-751 | NP18 | QP38 | QP38 | H | H | H | H | H | H | H |
| 1-752 | NP18 | QP39 | QP39 | H | H | H | H | H | H | H |
| 1-753 | NP18 | QP44 | QP44 | H | H | H | H | H | H | H |
| 1-754 | NP18 | QP45 | QP45 | H | H | H | H | H | H | H |
| 1-755 | NP18 | QP63 | QP63 | H | H | H | H | H | H | H |
| 1-756 | NP18 | QP64 | QP64 | H | H | H | H | H | H | H |
| 1-757 | NP18 | NP1 | NP1 | H | H | H | H | H | H | H |
| 1-758 | NP18 | NP3 | NP3 | H | H | H | H | H | H | H |
| 1-759 | NP18 | NP4 | NP4 | H | H | H | H | H | H | H |
| 1-760 | NP18 | NP5 | NP5 | H | H | H | H | H | H | H |
| 1-761 | NP18 | NP6 | NP6 | H | H | H | H | H | H | H |
| 1-762 | NP18 | NP7 | NP7 | H | H | H | H | H | H | H |
| 1-763 | NP18 | NP8 | NP8 | H | H | H | H | H | H | H |
| 1-764 | NP18 | NP11 | NP11 | H | H | H | H | H | H | H |
| 1-765 | NP18 | NP13 | NP13 | H | H | H | H | H | H | H |
| 1-766 | NP18 | NP14 | NP14 | H | H | H | H | H | H | H |
| 1-767 | NP18 | NP15 | NP15 | H | H | H | H | H | H | H |
| 1-768 | NP18 | NP16 | NP16 | H | H | H | H | H | H | H |
| 1-769 | NP18 | NP17 | NP17 | H | H | H | H | H | H | H |
| 1-770 | NP18 | NP18 | NP18 | H | H | H | H | H | H | H |
| 1-771 | NP18 | PN1 | PN1 | H | H | H | H | H | H | H |
| 1-772 | NP18 | FL1 | FL1 | H | H | H | H | H | H | H |
| 1-773 | NP18 | TH3 | TH3 | H | H | H | H | H | H | H |
| 1-774 | NP18 | BT1 | BT1 | H | H | H | H | H | H | H |
| 1-775 | NP18 | BT3 | BT3 | H | H | H | H | H | H | H |
| 1-776 | NP18 | BT6 | BT6 | H | H | H | H | H | H | H |
| 1-777 | NP18 | BT7 | BT7 | H | H | H | H | H | H | H |
| 1-778 | NP18 | CZ1 | CZ1 | H | H | H | H | H | H | H |
| 1-779 | NP18 | CZ6 | CZ6 | H | H | H | H | H | H | H |
| 1-780 | PN1 | P1 | P1 | H | H | H | H | H | H | H |
| 1-781 | PN1 | P2 | P2 | H | H | H | H | H | H | H |
| 1-782 | PN1 | P4 | P4 | H | H | H | H | H | H | H |

Table 2-18

| | | | | | | | | | | |
|---|---|---|---|---|---|---|---|---|---|---|
| 1-783 | PN1 | P5 | P5 | H | H | H | H | H | H | H |
| 1-784 | PN1 | P8 | P8 | H | H | H | H | H | H | H |
| 1-785 | PN1 | BP1 | BP1 | H | H | H | H | H | H | H |
| 1-786 | PN1 | BP2 | BP2 | H | H | H | H | H | H | H |
| 1-787 | PN1 | BP3 | BP3 | H | H | H | H | H | H | H |
| 1-788 | PN1 | BP4 | BP4 | H | H | H | H | H | H | H |
| 1-789 | PN1 | TP1 | TP1 | H | H | H | H | H | H | H |
| 1-790 | PN1 | TP4 | TP4 | H | H | H | H | H | H | H |
| 1-791 | PN1 | TP12 | TP12 | H | H | H | H | H | H | H |
| 1-792 | PN1 | TP15 | TP15 | H | H | H | H | H | H | H |
| 1-793 | PN1 | TP16 | TP16 | H | H | H | H | H | H | H |
| 1-794 | PN1 | TP17 | TP17 | H | H | H | H | H | H | H |
| 1-795 | PN1 | TP18 | TP18 | H | H | H | H | H | H | H |
| 1-796 | PN1 | QP38 | QP38 | H | H | H | H | H | H | H |
| 1-797 | PN1 | QP45 | QP45 | H | H | H | H | H | H | H |
| 1-798 | PN1 | QP64 | QP64 | H | H | H | H | H | H | H |
| 1-799 | PN1 | NP1 | NP1 | H | H | H | H | H | H | H |
| 1-800 | PN1 | NP3 | NP3 | H | H | H | H | H | H | H |
| 1-801 | PN1 | NP4 | NP4 | H | H | H | H | H | H | H |
| 1-802 | PN1 | NP5 | NP5 | H | H | H | H | H | H | H |
| 1-803 | PN1 | NP6 | NP6 | H | H | H | H | H | H | H |
| 1-804 | PN1 | NP7 | NP7 | H | H | H | H | H | H | H |
| 1-805 | PN1 | NP8 | NP8 | H | H | H | H | H | H | H |
| 1-806 | PN1 | NP11 | NP11 | H | H | H | H | H | H | H |
| 1-807 | PN1 | NP13 | NP13 | H | H | H | H | H | H | H |
| 1-808 | PN1 | NP14 | NP14 | H | H | H | H | H | H | H |
| 1-809 | PN1 | NP15 | NP15 | H | H | H | H | H | H | H |
| 1-810 | PN1 | NP16 | NP16 | H | H | H | H | H | H | H |
| 1-811 | PN1 | NP17 | NP17 | H | H | H | H | H | H | H |
| 1-812 | PN1 | NP18 | NP18 | H | H | H | H | H | H | H |
| 1-813 | PN1 | PN1 | PN1 | H | H | H | H | H | H | H |
| 1-814 | PN1 | FL1 | FL1 | H | H | H | H | H | H | H |
| 1-815 | PN1 | TH3 | TH3 | H | H | H | H | H | H | H |
| 1-816 | PN1 | BT1 | BT1 | H | H | H | H | H | H | H |
| 1-817 | PN1 | BT3 | BT3 | H | H | H | H | H | H | H |
| 1-818 | PN1 | CZ1 | CZ1 | H | H | H | H | H | H | H |
| 1-819 | PN1 | CZ6 | CZ6 | H | H | H | H | H | H | H |
| 1-820 | CS1 | P1 | P1 | H | H | H | H | H | H | H |
| 1-821 | CS1 | P2 | P2 | H | H | H | H | H | H | H |
| 1-822 | CS1 | P4 | P4 | H | H | H | H | H | H | H |
| 1-823 | CS1 | P5 | P5 | H | H | H | H | H | H | H |
| 1-824 | CS1 | P8 | P8 | H | H | H | H | H | H | H |
| 1-825 | CS1 | BP1 | BP1 | H | H | H | H | H | H | H |
| 1-826 | CS1 | BP2 | BP2 | H | H | H | H | H | H | H |
| 1-827 | CS1 | BP3 | BP3 | H | H | H | H | H | H | H |
| 1-828 | CS1 | BP4 | BP4 | H | H | H | H | H | H | H |

Table 2-19

| | | | | | | | | | | |
|---|---|---|---|---|---|---|---|---|---|---|
| 1-829 | CS1 | TP1 | TP1 | H | H | H | H | H | H | H |
| 1-830 | CS1 | TP4 | TP4 | H | H | H | H | H | H | H |
| 1-831 | CS1 | TP12 | TP12 | H | H | H | H | H | H | H |
| 1-832 | CS1 | TP15 | TP15 | H | H | H | H | H | H | H |
| 1-833 | CS1 | TP16 | TP16 | H | H | H | H | H | H | H |
| 1-834 | CS1 | TP17 | TP17 | H | H | H | H | H | H | H |
| 1-835 | CS1 | TP18 | TP18 | H | H | H | H | H | H | H |
| 1-836 | CS1 | QP38 | QP38 | H | H | H | H | H | H | H |
| 1-837 | CS1 | QP45 | QP45 | H | H | H | H | H | H | H |
| 1-838 | CS1 | QP64 | QP64 | H | H | H | H | H | H | H |
| 1-839 | CS1 | NP1 | NP1 | H | H | H | H | H | H | H |
| 1-840 | CS1 | NP3 | NP3 | H | H | H | H | H | H | H |
| 1-841 | CS1 | NP4 | NP4 | H | H | H | H | H | H | H |
| 1-842 | CS1 | NP5 | NP5 | H | H | H | H | H | H | H |
| 1-843 | CS1 | NP6 | NP6 | H | H | H | H | H | H | H |
| 1-844 | CS1 | NP7 | NP7 | H | H | H | H | H | H | H |
| 1-845 | CS1 | NP8 | NP8 | H | H | H | H | H | H | H |
| 1-846 | CS1 | NP11 | NP11 | H | H | H | H | H | H | H |
| 1-847 | CS1 | NP13 | NP13 | H | H | H | H | H | H | H |
| 1-848 | CS1 | NP14 | NP14 | H | H | H | H | H | H | H |
| 1-849 | CS1 | NP15 | NP15 | H | H | H | H | H | H | H |
| 1-850 | CS1 | NP16 | NP16 | H | H | H | H | H | H | H |
| 1-851 | CS1 | NP17 | NP17 | H | H | H | H | H | H | H |
| 1-852 | CS1 | NP18 | NP18 | H | H | H | H | H | H | H |
| 1-853 | CS1 | PN1 | PN1 | H | H | H | H | H | H | H |
| 1-854 | CS1 | FL1 | FL1 | H | H | H | H | H | H | H |
| 1-855 | CS1 | TH3 | TH3 | H | H | H | H | H | H | H |
| 1-856 | CS1 | BT1 | BT1 | H | H | H | H | H | H | H |
| 1-857 | CS1 | BT3 | BT3 | H | H | H | H | H | H | H |
| 1-858 | CS1 | CZ1 | CZ1 | H | H | H | H | H | H | H |
| 1-859 | CS1 | CZ6 | CZ6 | H | H | H | H | H | H | H |
| 1-860 | TPL1 | P1 | P1 | H | H | H | H | H | H | H |
| 1-861 | TPL1 | P2 | P2 | H | H | H | H | H | H | H |
| 1-862 | TPL1 | P4 | P4 | H | H | H | H | H | H | H |
| 1-863 | TPL1 | P5 | P5 | H | H | H | H | H | H | H |
| 1-864 | TPL1 | P8 | P8 | H | H | H | H | H | H | H |
| 1-865 | TPL1 | BP1 | BP1 | H | H | H | H | H | H | H |
| 1-866 | TPL1 | BP2 | BP2 | H | H | H | H | H | H | H |
| 1-867 | TPL1 | BP3 | BP3 | H | H | H | H | H | H | H |
| 1-868 | TPL1 | BP4 | BP4 | H | H | H | H | H | H | H |
| 1-869 | TPL1 | TP1 | TP1 | H | H | H | H | H | H | H |
| 1-870 | TPL1 | TP4 | TP4 | H | H | H | H | H | H | H |
| 1-871 | TPL1 | TP12 | TP12 | H | H | H | H | H | H | H |
| 1-872 | TPL1 | TP15 | TP15 | H | H | H | H | H | H | H |
| 1-873 | TPL1 | TP16 | TP16 | H | H | H | H | H | H | H |
| 1-874 | TPL1 | TP17 | TP17 | H | H | H | H | H | H | H |

Table 2-20

| | | | | | | | | | | |
|---|---|---|---|---|---|---|---|---|---|---|
| 1-875 | TPL1 | TP18 | TP18 | H | H | H | H | H | H | H |
| 1-876 | TPL1 | QP38 | QP38 | H | H | H | H | H | H | H |
| 1-877 | TPL1 | QP45 | QP45 | H | H | H | H | H | H | H |
| 1-878 | TPL1 | QP64 | QP64 | H | H | H | H | H | H | H |
| 1-879 | TPL1 | NP1 | NP1 | H | H | H | H | H | H | H |
| 1-880 | TPL1 | NP3 | NP3 | H | H | H | H | H | H | H |
| 1-881 | TPL1 | NP4 | NP4 | H | H | H | H | H | H | H |
| 1-882 | TPL1 | NP5 | NP5 | H | H | H | H | H | H | H |
| 1-883 | TPL1 | NP6 | NP6 | H | H | H | H | H | H | H |
| 1-884 | TPL1 | NP7 | NP7 | H | H | H | H | H | H | H |
| 1-885 | TPL1 | NP8 | NP8 | H | H | H | H | H | H | H |
| 1-886 | TPL1 | NP11 | NP11 | H | H | H | H | H | H | H |
| 1-887 | TPL1 | NP13 | NP13 | H | H | H | H | H | H | H |
| 1-888 | TPL1 | NP14 | NP14 | H | H | H | H | H | H | H |
| 1-889 | TPL1 | NP15 | NP15 | H | H | H | H | H | H | H |
| 1-890 | TPL1 | NP16 | NP16 | H | H | H | H | H | H | H |
| 1-891 | TPL1 | NP17 | NP17 | H | H | H | H | H | H | H |
| 1-892 | TPL1 | NP18 | NP18 | H | H | H | H | H | H | H |
| 1-893 | TPL1 | PN1 | PN1 | H | H | H | H | H | H | H |
| 1-894 | TPL1 | FL1 | FL1 | H | H | H | H | H | H | H |
| 1-895 | TPL1 | TH3 | TH3 | H | H | H | H | H | H | H |
| 1-896 | TPL1 | BT1 | BT1 | H | H | H | H | H | H | H |
| 1-897 | TPL1 | BT3 | BT3 | H | H | H | H | H | H | H |
| 1-898 | TPL1 | CZ1 | CZ1 | H | H | H | H | H | H | H |
| 1-899 | TPL1 | CZ6 | CZ6 | H | H | H | H | H | H | H |
| 1-900 | FL1 | P1 | P1 | H | H | H | H | H | H | H |
| 1-901 | FL1 | P2 | P2 | H | H | H | H | H | H | H |
| 1-902 | FL1 | P4 | P4 | H | H | H | H | H | H | H |
| 1-903 | FL1 | P5 | P5 | H | H | H | H | H | H | H |
| 1-904 | FL1 | P8 | P8 | H | H | H | H | H | H | H |
| 1-905 | FL1 | BP1 | BP1 | H | H | H | H | H | H | H |
| 1-906 | FL1 | BP2 | BP2 | H | H | H | H | H | H | H |
| 1-907 | FL1 | BP3 | BP3 | H | H | H | H | H | H | H |
| 1-908 | FL1 | BP4 | BP4 | H | H | H | H | H | H | H |
| 1-909 | FL1 | TP1 | TP1 | H | H | H | H | H | H | H |
| 1-910 | FL1 | TP4 | TP4 | H | H | H | H | H | H | H |
| 1-911 | FL1 | TP12 | TP12 | H | H | H | H | H | H | H |
| 1-912 | FL1 | TP15 | TP15 | H | H | H | H | H | H | H |
| 1-913 | FL1 | TP16 | TP16 | H | H | H | H | H | H | H |
| 1-914 | FL1 | TP17 | TP17 | H | H | H | H | H | H | H |
| 1-915 | FL1 | TP18 | TP18 | H | H | H | H | H | H | H |
| 1-916 | FL1 | QP38 | QP38 | H | H | H | H | H | H | H |
| 1-917 | FL1 | QP45 | QP45 | H | H | H | H | H | H | H |
| 1-918 | FL1 | QP64 | QP64 | H | H | H | H | H | H | H |
| 1-919 | FL1 | NP1 | NP1 | H | H | H | H | H | H | H |
| 1-920 | FL1 | NP3 | NP3 | H | H | H | H | H | H | H |

Table 2-21

| | | | | | | | | | | |
|---|---|---|---|---|---|---|---|---|---|---|
| 1-921 | FL1 | NP4 | NP4 | H | H | H | H | H | H | H |
| 1-922 | FL1 | NP5 | NP5 | H | H | H | H | H | H | H |
| 1-923 | FL1 | NP6 | NP6 | H | H | H | H | H | H | H |
| 1-924 | FL1 | NP7 | NP7 | H | H | H | H | H | H | H |
| 1-925 | FL1 | NP8 | NP8 | H | H | H | H | H | H | H |
| 1-926 | FL1 | NP11 | NP11 | H | H | H | H | H | H | H |
| 1-927 | FL1 | NP13 | NP13 | H | H | H | H | H | H | H |
| 1-928 | FL1 | NP14 | NP14 | H | H | H | H | H | H | H |
| 1-929 | FL1 | NP15 | NP15 | H | H | H | H | H | H | H |
| 1-930 | FL1 | NP16 | NP16 | H | H | H | H | H | H | H |
| 1-931 | FL1 | NP17 | NP17 | H | H | H | H | H | H | H |
| 1-932 | FL1 | NP18 | NP18 | H | H | H | H | H | H | H |
| 1-933 | FL1 | PN1 | PN1 | H | H | H | H | H | H | H |
| 1-934 | FL1 | FL1 | FL1 | H | H | H | H | H | H | H |
| 1-935 | FL1 | TH3 | TH3 | H | H | H | H | H | H | H |
| 1-936 | FL1 | BT1 | BT1 | H | H | H | H | H | H | H |
| 1-937 | FL1 | BT3 | BT3 | H | H | H | H | H | H | H |
| 1-938 | FL1 | CZ1 | CZ1 | H | H | H | H | H | H | H |
| 1-939 | FL1 | CZ6 | CZ6 | H | H | H | H | H | H | H |
| 1-940 | TH3 | P1 | P1 | H | H | H | H | H | H | H |
| 1-941 | TH3 | P2 | P2 | H | H | H | H | H | H | H |
| 1-942 | TH3 | P4 | P4 | H | H | H | H | H | H | H |
| 1-943 | TH3 | P5 | P5 | H | H | H | H | H | H | H |
| 1-944 | TH3 | P8 | P8 | H | H | H | H | H | H | H |
| 1-945 | TH3 | BP1 | BP1 | H | H | H | H | H | H | H |
| 1-946 | TH3 | BP2 | BP2 | H | H | H | H | H | H | H |
| 1-947 | TH3 | BP3 | BP3 | H | H | H | H | H | H | H |
| 1-948 | TH3 | BP4 | BP4 | H | H | H | H | H | H | H |
| 1-949 | TH3 | TP1 | TP1 | H | H | H | H | H | H | H |
| 1-950 | TH3 | TP4 | TP4 | H | H | H | H | H | H | H |
| 1-951 | TH3 | TP12 | TP12 | H | H | H | H | H | H | H |
| 1-952 | TH3 | TP15 | TP15 | H | H | H | H | H | H | H |
| 1-953 | TH3 | TP16 | TP16 | H | H | H | H | H | H | H |
| 1-954 | TH3 | TP17 | TP17 | H | H | H | H | H | H | H |
| 1-955 | TH3 | TP18 | TP18 | H | H | H | H | H | H | H |
| 1-956 | TH3 | QP38 | QP38 | H | H | H | H | H | H | H |
| 1-957 | TH3 | QP45 | QP45 | H | H | H | H | H | H | H |
| 1-958 | TH3 | QP64 | QP64 | H | H | H | H | H | H | H |
| 1-959 | TH3 | NP1 | NP1 | H | H | H | H | H | H | H |
| 1-960 | TH3 | NP3 | NP3 | H | H | H | H | H | H | H |
| 1-961 | TH3 | NP4 | NP4 | H | H | H | H | H | H | H |
| 1-962 | TH3 | NP5 | NP5 | H | H | H | H | H | H | H |
| 1-963 | TH3 | NP6 | NP6 | H | H | H | H | H | H | H |
| 1-964 | TH3 | NP7 | NP7 | H | H | H | H | H | H | H |
| 1-965 | TH3 | NP8 | NP8 | H | H | H | H | H | H | H |
| 1-966 | TH3 | NP11 | NP11 | H | H | H | H | H | H | H |

Table 2-22

| | | | | | | | | | | |
|---|---|---|---|---|---|---|---|---|---|---|
| 1-967 | TH3 | NP13 | NP13 | H | H | H | H | H | H | H |
| 1-968 | TH3 | NP14 | NP14 | H | H | H | H | H | H | H |
| 1-969 | TH3 | NP15 | NP15 | H | H | H | H | H | H | H |
| 1-970 | TH3 | NP16 | NP16 | H | H | H | H | H | H | H |
| 1-971 | TH3 | NP17 | NP17 | H | H | H | H | H | H | H |
| 1-972 | TH3 | NP18 | NP18 | H | H | H | H | H | H | H |
| 1-973 | TH3 | PN1 | PN1 | H | H | H | H | H | H | H |
| 1-974 | TH3 | FL1 | FL1 | H | H | H | H | H | H | H |
| 1-975 | TH3 | TH3 | TH3 | H | H | H | H | H | H | H |
| 1-976 | TH3 | BT1 | BT1 | H | H | H | H | H | H | H |
| 1-977 | TH3 | BT3 | BT3 | H | H | H | H | H | H | H |
| 1-978 | TH3 | CZ1 | CZ1 | H | H | H | H | H | H | H |
| 1-979 | TH3 | CZ6 | CZ6 | H | H | H | H | H | H | H |
| 1-980 | BT1 | P1 | P1 | H | H | H | H | H | H | H |
| 1-981 | BT1 | P2 | P2 | H | H | H | H | H | H | H |
| 1-982 | BT1 | P4 | P4 | H | H | H | H | H | H | H |
| 1-983 | BT1 | P5 | P5 | H | H | H | H | H | H | H |
| 1-984 | BT1 | P8 | P8 | H | H | H | H | H | H | H |
| 1-985 | BT1 | BP1 | BP1 | H | H | H | H | H | H | H |
| 1-986 | BT1 | BP2 | BP2 | H | H | H | H | H | H | H |
| 1-987 | BT1 | BP3 | BP3 | H | H | H | H | H | H | H |
| 1-988 | BT1 | BP4 | BP4 | H | H | H | H | H | H | H |
| 1-989 | BT1 | TP1 | TP1 | H | H | H | H | H | H | H |
| 1-990 | BT1 | TP4 | TP4 | H | H | H | H | H | H | H |
| 1-991 | BT1 | TP12 | TP12 | H | H | H | H | H | H | H |
| 1-992 | BT1 | TP15 | TP15 | H | H | H | H | H | H | H |
| 1-993 | BT1 | TP16 | TP16 | H | H | H | H | H | H | H |
| 1-994 | BT1 | TP17 | TP17 | H | H | H | H | H | H | H |
| 1-995 | BT1 | TP18 | TP18 | H | H | H | H | H | H | H |
| 1-996 | BT1 | QP38 | QP38 | H | H | H | H | H | H | H |
| 1-997 | BT1 | QP45 | QP45 | H | H | H | H | H | H | H |
| 1-998 | BT1 | QP64 | QP64 | H | H | H | H | H | H | H |
| 1-999 | BT1 | NP1 | NP1 | H | H | H | H | H | H | H |
| 1-1000 | BT1 | NP3 | NP3 | H | H | H | H | H | H | H |
| 1-1001 | BT1 | NP4 | NP4 | H | H | H | H | H | H | H |
| 1-1002 | BT1 | NP5 | NP5 | H | H | H | H | H | H | H |
| 1-1003 | BT1 | NP6 | NP6 | H | H | H | H | H | H | H |
| 1-1004 | BT1 | NP7 | NP7 | H | H | H | H | H | H | H |
| 1-1005 | BT1 | NP8 | NP8 | H | H | H | H | H | H | H |
| 1-1006 | BT1 | NP11 | NP11 | H | H | H | H | H | H | H |
| 1-1007 | BT1 | NP13 | NP13 | H | H | H | H | H | H | H |
| 1-1008 | BT1 | NP14 | NP14 | H | H | H | H | H | H | H |
| 1-1009 | BT1 | NP15 | NP15 | H | H | H | H | H | H | H |
| 1-1010 | BT1 | NP16 | NP16 | H | H | H | H | H | H | H |
| 1-1011 | BT1 | NP17 | NP17 | H | H | H | H | H | H | H |
| 1-1012 | BT1 | NP18 | NP18 | H | H | H | H | H | H | H |

Table 2-23

| | | | | | | | | | | |
|---|---|---|---|---|---|---|---|---|---|---|
| 1-1013 | BT1 | PN1 | PN1 | H | H | H | H | H | H | H |
| 1-1014 | BT1 | FL1 | FL1 | H | H | H | H | H | H | H |
| 1-1015 | BT1 | TH3 | TH3 | H | H | H | H | H | H | H |
| 1-1016 | BT1 | BT1 | BT1 | H | H | H | H | H | H | H |
| 1-1017 | BT1 | BT3 | BT3 | H | H | H | H | H | H | H |
| 1-1018 | BT1 | CZ1 | CZ1 | H | H | H | H | H | H | H |
| 1-1019 | BT1 | CZ6 | CZ6 | H | H | H | H | H | H | H |
| 1-1020 | BT3 | P1 | P1 | H | H | H | H | H | H | H |
| 1-1021 | BT3 | P2 | P2 | H | H | H | H | H | H | H |
| 1-1022 | BT3 | P4 | P4 | H | H | H | H | H | H | H |
| 1-1023 | BT3 | P5 | P5 | H | H | H | H | H | H | H |
| 1-1024 | BT3 | P8 | P8 | H | H | H | H | H | H | H |
| 1-1025 | BT3 | BP1 | BP1 | H | H | H | H | H | H | H |
| 1-1026 | BT3 | BP2 | BP2 | H | H | H | H | H | H | H |
| 1-1027 | BT3 | BP3 | BP3 | H | H | H | H | H | H | H |
| 1-1028 | BT3 | BP4 | BP4 | H | H | H | H | H | H | H |
| 1-1029 | BT3 | TP1 | TP1 | H | H | H | H | H | H | H |
| 1-1030 | BT3 | TP4 | TP4 | H | H | H | H | H | H | H |
| 1-1031 | BT3 | TP12 | TP12 | H | H | H | H | H | H | H |
| 1-1032 | BT3 | TP15 | TP15 | H | H | H | H | H | H | H |
| 1-1033 | BT3 | TP16 | TP16 | H | H | H | H | H | H | H |
| 1-1034 | BT3 | TP17 | TP17 | H | H | H | H | H | H | H |
| 1-1035 | BT3 | TP18 | TP18 | H | H | H | H | H | H | H |
| 1-1036 | BT3 | QP38 | QP38 | H | H | H | H | H | H | H |
| 1-1037 | BT3 | QP45 | QP45 | H | H | H | H | H | H | H |
| 1-1038 | BT3 | QP64 | QP64 | H | H | H | H | H | H | H |
| 1-1039 | BT3 | NP1 | NP1 | H | H | H | H | H | H | H |
| 1-1040 | BT3 | NP3 | NP3 | H | H | H | H | H | H | H |
| 1-1041 | BT3 | NP4 | NP4 | H | H | H | H | H | H | H |
| 1-1042 | BT3 | NP5 | NP5 | H | H | H | H | H | H | H |
| 1-1043 | BT3 | NP6 | NP6 | H | H | H | H | H | H | H |
| 1-1044 | BT3 | NP7 | NP7 | H | H | H | H | H | H | H |
| 1-1045 | BT3 | NP8 | NP8 | H | H | H | H | H | H | H |
| 1-1046 | BT3 | NP11 | NP11 | H | H | H | H | H | H | H |
| 1-1047 | BT3 | NP13 | NP13 | H | H | H | H | H | H | H |
| 1-1048 | BT3 | NP14 | NP14 | H | H | H | H | H | H | H |
| 1-1049 | BT3 | NP15 | NP15 | H | H | H | H | H | H | H |
| 1-1050 | BT3 | NP16 | NP16 | H | H | H | H | H | H | H |
| 1-1051 | BT3 | NP17 | NP17 | H | H | H | H | H | H | H |
| 1-1052 | BT3 | NP18 | NP18 | H | H | H | H | H | H | H |
| 1-1053 | BT3 | PN1 | PN1 | H | H | H | H | H | H | H |
| 1-1054 | BT3 | FL1 | FL1 | H | H | H | H | H | H | H |
| 1-1055 | BT3 | TH3 | TH3 | H | H | H | H | H | H | H |
| 1-1056 | BT3 | BT1 | BT1 | H | H | H | H | H | H | H |
| 1-1057 | BT3 | BT3 | BT3 | H | H | H | H | H | H | H |
| 1-1058 | BT3 | CZ1 | CZ1 | H | H | H | H | H | H | H |

Table 2-24

| | | | | | | | | | | |
|---|---|---|---|---|---|---|---|---|---|---|
| 1-1059 | CZ1 | P1 | P1 | H | H | H | H | H | H | H |
| 1-1060 | CZ1 | P2 | P2 | H | H | H | H | H | H | H |
| 1-1061 | CZ1 | P4 | P4 | H | H | H | H | H | H | H |
| 1-1062 | CZ1 | P5 | P5 | H | H | H | H | H | H | H |
| 1-1063 | CZ1 | P8 | P8 | H | H | H | H | H | H | H |
| 1-1064 | CZ1 | BP1 | BP1 | H | H | H | H | H | H | H |
| 1-1065 | CZ1 | BP2 | BP2 | H | H | H | H | H | H | H |
| 1-1066 | CZ1 | BP3 | BP3 | H | H | H | H | H | H | H |
| 1-1067 | CZ1 | BP4 | BP4 | H | H | H | H | H | H | H |
| 1-1068 | CZ1 | TP1 | TP1 | H | H | H | H | H | H | H |
| 1-1069 | CZ1 | TP4 | TP4 | H | H | H | H | H | H | H |
| 1-1070 | CZ1 | TP12 | TP12 | H | H | H | H | H | H | H |
| 1-1071 | CZ1 | TP15 | TP15 | H | H | H | H | H | H | H |
| 1-1072 | CZ1 | TP16 | TP16 | H | H | H | H | H | H | H |
| 1-1073 | CZ1 | TP17 | TP17 | H | H | H | H | H | H | H |
| 1-1074 | CZ1 | TP18 | TP18 | H | H | H | H | H | H | H |
| 1-1075 | CZ1 | QP38 | QP38 | H | H | H | H | H | H | H |
| 1-1076 | CZ1 | QP45 | QP45 | H | H | H | H | H | H | H |
| 1-1077 | CZ1 | QP64 | QP64 | H | H | H | H | H | H | H |
| 1-1078 | CZ1 | NP1 | NP1 | H | H | H | H | H | H | H |
| 1-1079 | CZ1 | NP3 | NP3 | H | H | H | H | H | H | H |
| 1-1080 | CZ1 | NP4 | NP4 | H | H | H | H | H | H | H |
| 1-1081 | CZ1 | NP5 | NP5 | H | H | H | H | H | H | H |
| 1-1082 | CZ1 | NP6 | NP6 | H | H | H | H | H | H | H |
| 1-1083 | CZ1 | NP7 | NP7 | H | H | H | H | H | H | H |
| 1-1084 | CZ1 | NP8 | NP8 | H | H | H | H | H | H | H |
| 1-1085 | CZ1 | NP11 | NP11 | H | H | H | H | H | H | H |
| 1-1086 | CZ1 | NP13 | NP13 | H | H | H | H | H | H | H |
| 1-1087 | CZ1 | NP14 | NP14 | H | H | H | H | H | H | H |
| 1-1088 | CZ1 | NP15 | NP15 | H | H | H | H | H | H | H |
| 1-1089 | CZ1 | NP16 | NP16 | H | H | H | H | H | H | H |
| 1-1090 | CZ1 | NP17 | NP17 | H | H | H | H | H | H | H |
| 1-1091 | CZ1 | NP18 | NP18 | H | H | H | H | H | H | H |
| 1-1092 | CZ1 | PN1 | PN1 | H | H | H | H | H | H | H |
| 1-1093 | CZ1 | FL1 | FL1 | H | H | H | H | H | H | H |
| 1-1094 | CZ1 | TH3 | TH3 | H | H | H | H | H | H | H |
| 1-1095 | CZ1 | BT1 | BT1 | H | H | H | H | H | H | H |
| 1-1096 | CZ1 | BT3 | BT3 | H | H | H | H | H | H | H |
| 1-1097 | CZ1 | CZ1 | CZ1 | H | H | H | H | H | H | H |
| 1-1098 | TP1 | P1 | P1 | H | H | H | H | H | H | Me |
| 1-1099 | TP1 | P2 | P2 | H | H | H | H | H | H | Me |
| 1-1100 | TP1 | P4 | P4 | H | H | H | H | H | H | Me |
| 1-1101 | TP1 | P5 | P5 | H | H | H | H | H | H | Me |
| 1-1102 | TP1 | P8 | P8 | H | H | H | H | H | H | Me |
| 1-1103 | TP1 | BP1 | BP1 | H | H | H | H | H | H | Me |
| 1-1104 | TP1 | BP2 | BP2 | H | H | H | H | H | H | Me |

Table 2-25

| | | | | | | | | | | |
|---|---|---|---|---|---|---|---|---|---|---|
| 1-1105 | TP1 | BP3 | BP3 | H | H | H | H | H | H | Me |
| 1-1106 | TP1 | BP4 | BP4 | H | H | H | H | H | H | Me |
| 1-1107 | TP1 | BP7 | BP7 | H | H | H | H | H | H | Me |
| 1-1108 | TP1 | TP1 | TP1 | H | H | H | H | H | H | Me |
| 1-1109 | TP1 | TP4 | TP4 | H | H | H | H | H | H | Me |
| 1-1110 | TP1 | TP12 | TP12 | H | H | H | H | H | H | Me |
| 1-1111 | TP1 | TP15 | TP15 | H | H | H | H | H | H | Me |
| 1-1112 | TP1 | TP16 | TP16 | H | H | H | H | H | H | Me |
| 1-1113 | TP1 | TP17 | TP17 | H | H | H | H | H | H | Me |
| 1-1114 | TP1 | TP18 | TP18 | H | H | H | H | H | H | Me |
| 1-1115 | TP1 | QP38 | QP38 | H | H | H | H | H | H | Me |
| 1-1116 | TP1 | QP44 | QP44 | H | H | H | H | H | H | Me |
| 1-1117 | TP1 | QP64 | QP64 | H | H | H | H | H | H | Me |
| 1-1118 | TP1 | NP1 | NP1 | H | H | H | H | H | H | Me |
| 1-1119 | TP1 | NP3 | NP3 | H | H | H | H | H | H | Me |
| 1-1120 | TP1 | NP4 | NP4 | H | H | H | H | H | H | Me |
| 1-1121 | TP1 | NP5 | NP5 | H | H | H | H | H | H | Me |
| 1-1122 | TP1 | NP6 | NP6 | H | H | H | H | H | H | Me |
| 1-1123 | TP1 | NP7 | NP7 | H | H | H | H | H | H | Me |
| 1-1124 | TP1 | NP8 | NP8 | H | H | H | H | H | H | Me |
| 1-1125 | TP1 | NP11 | NP11 | H | H | H | H | H | H | Me |
| 1-1126 | TP1 | NP13 | NP13 | H | H | H | H | H | H | Me |
| 1-1127 | TP1 | NP14 | NP14 | H | H | H | H | H | H | Me |
| 1-1128 | TP1 | NP15 | NP15 | H | H | H | H | H | H | Me |
| 1-1129 | TP1 | NP16 | NP16 | H | H | H | H | H | H | Me |
| 1-1130 | TP1 | NP17 | NP17 | H | H | H | H | H | H | Me |
| 1-1131 | TP1 | NP18 | NP18 | H | H | H | H | H | H | Me |
| 1-1132 | TP1 | PN1 | PN1 | H | H | H | H | H | H | Me |
| 1-1133 | TP1 | FL1 | FL1 | H | H | H | H | H | H | Me |
| 1-1134 | TP1 | TH3 | TH3 | H | H | H | H | H | H | Me |
| 1-1135 | TP1 | BT1 | BT1 | H | H | H | H | H | H | Me |
| 1-1136 | TP1 | BT3 | BT3 | H | H | H | H | H | H | Me |
| 1-1137 | TP1 | BT6 | BT6 | H | H | H | H | H | H | Me |
| 1-1138 | TP1 | BT7 | BT7 | H | H | H | H | H | H | Me |
| 1-1139 | TP1 | CZ1 | CZ1 | H | H | H | H | H | H | Me |
| 1-1140 | TP16 | P1 | P1 | H | H | H | H | H | H | Me |
| 1-1141 | TP16 | P2 | P2 | H | H | H | H | H | H | Me |
| 1-1142 | TP16 | P4 | P4 | H | H | H | H | H | H | Me |
| 1-1143 | TP16 | P5 | P5 | H | H | H | H | H | H | Me |
| 1-1144 | TP16 | P8 | P8 | H | H | H | H | H | H | Me |
| 1-1145 | TP16 | BP1 | BP1 | H | H | H | H | H | H | Me |
| 1-1146 | TP16 | BP2 | BP2 | H | H | H | H | H | H | Me |
| 1-1147 | TP16 | BP3 | BP3 | H | H | H | H | H | H | Me |
| 1-1148 | TP16 | BP4 | BP4 | H | H | H | H | H | H | Me |
| 1-1149 | TP16 | BP7 | BP7 | H | H | H | H | H | H | Me |
| 1-1150 | TP16 | TP1 | TP1 | H | H | H | H | H | H | Me |

Table 2-26

| | | | | | | | | | | |
|---|---|---|---|---|---|---|---|---|---|---|
| 1-1151 | TP16 | TP4 | TP4 | H | H | H | H | H | H | Me |
| 1-1152 | TP16 | TP12 | TP12 | H | H | H | H | H | H | Me |
| 1-1153 | TP16 | TP15 | TP15 | H | H | H | H | H | H | Me |
| 1-1154 | TP16 | TP16 | TP16 | H | H | H | H | H | H | Me |
| 1-1155 | TP16 | TP17 | TP17 | H | H | H | H | H | H | Me |
| 1-1156 | TP16 | TP18 | TP18 | H | H | H | H | H | H | Me |
| 1-1157 | TP16 | QP38 | QP38 | H | H | H | H | H | H | Me |
| 1-1158 | TP16 | QP44 | QP44 | H | H | H | H | H | H | Me |
| 1-1159 | TP16 | QP64 | QP64 | H | H | H | H | H | H | Me |
| 1-1160 | TP16 | NP1 | NP1 | H | H | H | H | H | H | Me |
| 1-1161 | TP16 | NP3 | NP3 | H | H | H | H | H | H | Me |
| 1-1162 | TP16 | NP4 | NP4 | H | H | H | H | H | H | Me |
| 1-1163 | TP16 | NP5 | NP5 | H | H | H | H | H | H | Me |
| 1-1164 | TP16 | NP6 | NP6 | H | H | H | H | H | H | Me |
| 1-1165 | TP16 | NP7 | NP7 | H | H | H | H | H | H | Me |
| 1-1166 | TP16 | NP8 | NP8 | H | H | H | H | H | H | Me |
| 1-1167 | TP16 | NP11 | NP11 | H | H | H | H | H | H | Me |
| 1-1168 | TP16 | NP13 | NP13 | H | H | H | H | H | H | Me |
| 1-1169 | TP16 | NP14 | NP14 | H | H | H | H | H | H | Me |
| 1-1170 | TP16 | NP15 | NP15 | H | H | H | H | H | H | Me |
| 1-1171 | TP16 | NP16 | NP16 | H | H | H | H | H | H | Me |
| 1-1172 | TP16 | NP17 | NP17 | H | H | H | H | H | H | Me |
| 1-1173 | TP16 | NP18 | NP18 | H | H | H | H | H | H | Me |
| 1-1174 | TP16 | PN1 | PN1 | H | H | H | H | H | H | Me |
| 1-1175 | TP16 | FL1 | FL1 | H | H | H | H | H | H | Me |
| 1-1176 | TP16 | TH3 | TH3 | H | H | H | H | H | H | Me |
| 1-1177 | TP16 | BT1 | BT1 | H | H | H | H | H | H | Me |
| 1-1178 | TP16 | BT3 | BT3 | H | H | H | H | H | H | Me |
| 1-1179 | TP16 | BT6 | BT6 | H | H | H | H | H | H | Me |
| 1-1180 | TP16 | BT7 | BT7 | H | H | H | H | H | H | Me |
| 1-1181 | TP16 | CZ1 | CZ1 | H | H | H | H | H | H | Me |
| 1-1182 | NP11 | P1 | P1 | H | H | H | H | H | H | Me |
| 1-1183 | NP11 | P2 | P2 | H | H | H | H | H | H | Me |
| 1-1184 | NP11 | P4 | P4 | H | H | H | H | H | H | Me |
| 1-1185 | NP11 | P5 | P5 | H | H | H | H | H | H | Me |
| 1-1186 | NP11 | P8 | P8 | H | H | H | H | H | H | Me |
| 1-1187 | NP11 | BP1 | BP1 | H | H | H | H | H | H | Me |
| 1-1188 | NP11 | BP2 | BP2 | H | H | H | H | H | H | Me |
| 1-1189 | NP11 | BP3 | BP3 | H | H | H | H | H | H | Me |
| 1-1190 | NP11 | BP4 | BP4 | H | H | H | H | H | H | Me |
| 1-1191 | NP11 | BP7 | BP7 | H | H | H | H | H | H | Me |
| 1-1192 | NP11 | TP1 | TP1 | H | H | H | H | H | H | Me |
| 1-1193 | NP11 | TP4 | TP4 | H | H | H | H | H | H | Me |
| 1-1194 | NP11 | TP12 | TP12 | H | H | H | H | H | H | Me |
| 1-1195 | NP11 | TP15 | TP15 | H | H | H | H | H | H | Me |
| 1-1196 | NP11 | TP16 | TP16 | H | H | H | H | H | H | Me |

Table 2-27

| | | | | | | | | | | |
|---|---|---|---|---|---|---|---|---|---|---|
| 1-1197 | NP11 | TP17 | TP17 | H | H | H | H | H | H | Me |
| 1-1198 | NP11 | TP18 | TP18 | H | H | H | H | H | H | Me |
| 1-1199 | NP11 | QP38 | QP38 | H | H | H | H | H | H | Me |
| 1-1200 | NP11 | QP44 | QP44 | H | H | H | H | H | H | Me |
| 1-1201 | NP11 | QP64 | QP64 | H | H | H | H | H | H | Me |
| 1-1202 | NP11 | NP1 | NP1 | H | H | H | H | H | H | Me |
| 1-1203 | NP11 | NP3 | NP3 | H | H | H | H | H | H | Me |
| 1-1204 | NP11 | NP4 | NP4 | H | H | H | H | H | H | Me |
| 1-1205 | NP11 | NP5 | NP5 | H | H | H | H | H | H | Me |
| 1-1206 | NP11 | NP6 | NP6 | H | H | H | H | H | H | Me |
| 1-1207 | NP11 | NP7 | NP7 | H | H | H | H | H | H | Me |
| 1-1208 | NP11 | NP8 | NP8 | H | H | H | H | H | H | Me |
| 1-1209 | NP11 | NP11 | NP11 | H | H | H | H | H | H | Me |
| 1-1210 | NP11 | NP13 | NP13 | H | H | H | H | H | H | Me |
| 1-1211 | NP11 | NP14 | NP14 | H | H | H | H | H | H | Me |
| 1-1212 | NP11 | NP15 | NP15 | H | H | H | H | H | H | Me |
| 1-1213 | NP11 | NP16 | NP16 | H | H | H | H | H | H | Me |
| 1-1214 | NP11 | NP17 | NP17 | H | H | H | H | H | H | Me |
| 1-1215 | NP11 | NP18 | NP18 | H | H | H | H | H | H | Me |
| 1-1216 | NP11 | PN1 | PN1 | H | H | H | H | H | H | Me |
| 1-1217 | NP11 | FL1 | FL1 | H | H | H | H | H | H | Me |
| 1-1218 | NP11 | TH3 | TH3 | H | H | H | H | H | H | Me |
| 1-1219 | NP11 | BT1 | BT1 | H | H | H | H | H | H | Me |
| 1-1220 | NP11 | BT3 | BT3 | H | H | H | H | H | H | Me |
| 1-1221 | NP11 | BT6 | BT6 | H | H | H | H | H | H | Me |
| 1-1222 | NP11 | BT7 | BT7 | H | H | H | H | H | H | Me |
| 1-1223 | NP11 | CZ1 | CZ1 | H | H | H | H | H | H | Me |
| 1-1224 | BT1 | P1 | P1 | H | H | H | H | H | H | Me |
| 1-1225 | BT1 | P2 | P2 | H | H | H | H | H | H | Me |
| 1-1226 | BT1 | P4 | P4 | H | H | H | H | H | H | Me |
| 1-1227 | BT1 | P5 | P5 | H | H | H | H | H | H | Me |
| 1-1228 | BT1 | P8 | P8 | H | H | H | H | H | H | Me |
| 1-1229 | BT1 | BP1 | BP1 | H | H | H | H | H | H | Me |
| 1-1230 | BT1 | BP2 | BP2 | H | H | H | H | H | H | Me |
| 1-1231 | BT1 | BP3 | BP3 | H | H | H | H | H | H | Me |
| 1-1232 | BT1 | BP4 | BP4 | H | H | H | H | H | H | Me |
| 1-1233 | BT1 | BP7 | BP7 | H | H | H | H | H | H | Me |
| 1-1234 | BT1 | TP1 | TP1 | H | H | H | H | H | H | Me |
| 1-1235 | BT1 | TP4 | TP4 | H | H | H | H | H | H | Me |
| 1-1236 | BT1 | TP12 | TP12 | H | H | H | H | H | H | Me |
| 1-1237 | BT1 | TP15 | TP15 | H | H | H | H | H | H | Me |
| 1-1238 | BT1 | TP16 | TP16 | H | H | H | H | H | H | Me |
| 1-1239 | BT1 | TP17 | TP17 | H | H | H | H | H | H | Me |
| 1-1240 | BT1 | TP18 | TP18 | H | H | H | H | H | H | Me |
| 1-1241 | BT1 | QP38 | QP38 | H | H | H | H | H | H | Me |
| 1-1242 | BT1 | QP44 | QP44 | H | H | H | H | H | H | Me |

Table 2-28

| | | | | | | | | | | |
|---|---|---|---|---|---|---|---|---|---|---|
| 1-1243 | BT1 | QP64 | QP64 | H | H | H | H | H | H | Me |
| 1-1244 | BT1 | NP1 | NP1 | H | H | H | H | H | H | Me |
| 1-1245 | BT1 | NP3 | NP3 | H | H | H | H | H | H | Me |
| 1-1246 | BT1 | NP4 | NP4 | H | H | H | H | H | H | Me |
| 1-1247 | BT1 | NP5 | NP5 | H | H | H | H | H | H | Me |
| 1-1248 | BT1 | NP6 | NP6 | H | H | H | H | H | H | Me |
| 1-1249 | BT1 | NP7 | NP7 | H | H | H | H | H | H | Me |
| 1-1250 | BT1 | NP8 | NP8 | H | H | H | H | H | H | Me |
| 1-1251 | BT1 | NP11 | NP11 | H | H | H | H | H | H | Me |
| 1-1252 | BT1 | NP13 | NP13 | H | H | H | H | H | H | Me |
| 1-1253 | BT1 | NP14 | NP14 | H | H | H | H | H | H | Me |
| 1-1254 | BT1 | NP15 | NP15 | H | H | H | H | H | H | Me |
| 1-1255 | BT1 | NP16 | NP16 | H | H | H | H | H | H | Me |
| 1-1256 | BT1 | NP17 | NP17 | H | H | H | H | H | H | Me |
| 1-1257 | BT1 | NP18 | NP18 | H | H | H | H | H | H | Me |
| 1-1258 | BT1 | PN1 | PN1 | H | H | H | H | H | H | Me |
| 1-1259 | BT1 | FL1 | FL1 | H | H | H | H | H | H | Me |
| 1-1260 | BT1 | TH3 | TH3 | H | H | H | H | H | H | Me |
| 1-1261 | BT1 | BT1 | BT1 | H | H | H | H | H | H | Me |
| 1-1262 | BT1 | BT3 | BT3 | H | H | H | H | H | H | Me |
| 1-1263 | BT1 | BT6 | BT6 | H | H | H | H | H | H | Me |
| 1-1264 | BT1 | BT7 | BT7 | H | H | H | H | H | H | Me |
| 1-1265 | BT1 | CZ1 | CZ1 | H | H | H | H | H | H | Me |
| 1-1266 | BT3 | P1 | P1 | H | H | H | H | H | H | Me |
| 1-1267 | BT3 | P2 | P2 | H | H | H | H | H | H | Me |
| 1-1268 | BT3 | P4 | P4 | H | H | H | H | H | H | Me |
| 1-1269 | BT3 | P5 | P5 | H | H | H | H | H | H | Me |
| 1-1270 | BT3 | P8 | P8 | H | H | H | H | H | H | Me |
| 1-1271 | BT3 | BP1 | BP1 | H | H | H | H | H | H | Me |
| 1-1272 | BT3 | BP2 | BP2 | H | H | H | H | H | H | Me |
| 1-1273 | BT3 | BP3 | BP3 | H | H | H | H | H | H | Me |
| 1-1274 | BT3 | BP4 | BP4 | H | H | H | H | H | H | Me |
| 1-1275 | BT3 | BP7 | BP7 | H | H | H | H | H | H | Me |
| 1-1276 | BT3 | TP1 | TP1 | H | H | H | H | H | H | Me |
| 1-1277 | BT3 | TP4 | TP4 | H | H | H | H | H | H | Me |
| 1-1278 | BT3 | TP12 | TP12 | H | H | H | H | H | H | Me |
| 1-1279 | BT3 | TP15 | TP15 | H | H | H | H | H | H | Me |
| 1-1280 | BT3 | TP16 | TP16 | H | H | H | H | H | H | Me |
| 1-1281 | BT3 | TP17 | TP17 | H | H | H | H | H | H | Me |
| 1-1282 | BT3 | TP18 | TP18 | H | H | H | H | H | H | Me |
| 1-1283 | BT3 | QP38 | QP38 | H | H | H | H | H | H | Me |
| 1-1284 | BT3 | QP44 | QP44 | H | H | H | H | H | H | Me |
| 1-1285 | BT3 | QP64 | QP64 | H | H | H | H | H | H | Me |
| 1-1286 | BT3 | NP1 | NP1 | H | H | H | H | H | H | Me |
| 1-1287 | BT3 | NP3 | NP3 | H | H | H | H | H | H | Me |
| 1-1288 | BT3 | NP4 | NP4 | H | H | H | H | H | H | Me |

Table 2-29

| | | | | | | | | | | |
|---|---|---|---|---|---|---|---|---|---|---|
| 1-1289 | BT3 | NP5 | NP5 | H | H | H | H | H | H | Me |
| 1-1290 | BT3 | NP6 | NP6 | H | H | H | H | H | H | Me |
| 1-1291 | BT3 | NP7 | NP7 | H | H | H | H | H | H | Me |
| 1-1292 | BT3 | NP8 | NP8 | H | H | H | H | H | H | Me |
| 1-1293 | BT3 | NP11 | NP11 | H | H | H | H | H | H | Me |
| 1-1294 | BT3 | NP13 | NP13 | H | H | H | H | H | H | Me |
| 1-1295 | BT3 | NP14 | NP14 | H | H | H | H | H | H | Me |
| 1-1296 | BT3 | NP15 | NP15 | H | H | H | H | H | H | Me |
| 1-1297 | BT3 | NP16 | NP16 | H | H | H | H | H | H | Me |
| 1-1298 | BT3 | NP17 | NP17 | H | H | H | H | H | H | Me |
| 1-1299 | BT3 | NP18 | NP18 | H | H | H | H | H | H | Me |
| 1-1300 | BT3 | PN1 | PN1 | H | H | H | H | H | H | Me |
| 1-1301 | BT3 | FL1 | FL1 | H | H | H | H | H | H | Me |
| 1-1302 | BT3 | TH3 | TH3 | H | H | H | H | H | H | Me |
| 1-1303 | BT3 | BT1 | BT1 | H | H | H | H | H | H | Me |
| 1-1304 | BT3 | BT3 | BT3 | H | H | H | H | H | H | Me |
| 1-1305 | BT3 | BT6 | BT6 | H | H | H | H | H | H | Me |
| 1-1306 | BT3 | BT7 | BT7 | H | H | H | H | H | H | Me |
| 1-1307 | BT3 | CZ1 | CZ1 | H | H | H | H | H | H | Me |
| 1-1308 | CZ1 | P2 | P2 | H | H | H | H | H | H | Me |
| 1-1309 | CZ1 | P4 | P4 | H | H | H | H | H | H | Me |
| 1-1310 | CZ1 | P5 | P5 | H | H | H | H | H | H | Me |
| 1-1311 | CZ1 | P8 | P8 | H | H | H | H | H | H | Me |
| 1-1312 | CZ1 | BP1 | BP1 | H | H | H | H | H | H | Me |
| 1-1313 | CZ1 | BP2 | BP2 | H | H | H | H | H | H | Me |
| 1-1314 | CZ1 | BP3 | BP3 | H | H | H | H | H | H | Me |
| 1-1315 | CZ1 | BP4 | BP4 | H | H | H | H | H | H | Me |
| 1-1316 | CZ1 | BP7 | BP7 | H | H | H | H | H | H | Me |
| 1-1317 | CZ1 | TP1 | TP1 | H | H | H | H | H | H | Me |
| 1-1318 | CZ1 | TP4 | TP4 | H | H | H | H | H | H | Me |
| 1-1319 | CZ1 | TP12 | TP12 | H | H | H | H | H | H | Me |
| 1-1320 | CZ1 | TP15 | TP15 | H | H | H | H | H | H | Me |
| 1-1321 | CZ1 | TP16 | TP16 | H | H | H | H | H | H | Me |
| 1-1322 | CZ1 | TP17 | TP17 | H | H | H | H | H | H | Me |
| 1-1323 | CZ1 | TP18 | TP18 | H | H | H | H | H | H | Me |
| 1-1324 | CZ1 | QP38 | QP38 | H | H | H | H | H | H | Me |
| 1-1325 | CZ1 | QP44 | QP44 | H | H | H | H | H | H | Me |
| 1-1326 | CZ1 | QP64 | QP64 | H | H | H | H | H | H | Me |
| 1-1327 | CZ1 | NP1 | NP1 | H | H | H | H | H | H | Me |
| 1-1328 | CZ1 | NP3 | NP3 | H | H | H | H | H | H | Me |
| 1-1329 | CZ1 | NP4 | NP4 | H | H | H | H | H | H | Me |
| 1-1330 | CZ1 | NP5 | NP5 | H | H | H | H | H | H | Me |
| 1-1331 | CZ1 | NP6 | NP6 | H | H | H | H | H | H | Me |
| 1-1332 | CZ1 | NP7 | NP7 | H | H | H | H | H | H | Me |
| 1-1333 | CZ1 | NP8 | NP8 | H | H | H | H | H | H | Me |
| 1-1334 | CZ1 | NP11 | NP11 | H | H | H | H | H | H | Me |

Table 2-30

| | | | | | | | | | | |
|---|---|---|---|---|---|---|---|---|---|---|
| 1-1335 | CZ1 | NP13 | NP13 | H | H | H | H | H | H | Me |
| 1-1336 | CZ1 | NP14 | NP14 | H | H | H | H | H | H | Me |
| 1-1337 | CZ1 | NP15 | NP15 | H | H | H | H | H | H | Me |
| 1-1338 | CZ1 | NP16 | NP16 | H | H | H | H | H | H | Me |
| 1-1339 | CZ1 | NP17 | NP17 | H | H | H | H | H | H | Me |
| 1-1340 | CZ1 | NP18 | NP18 | H | H | H | H | H | H | Me |
| 1-1341 | CZ1 | PN1 | PN1 | H | H | H | H | H | H | Me |
| 1-1342 | CZ1 | FL1 | FL1 | H | H | H | H | H | H | Me |
| 1-1343 | CZ1 | TH3 | TH3 | H | H | H | H | H | H | Me |
| 1-1344 | CZ1 | BT1 | BT1 | H | H | H | H | H | H | Me |
| 1-1345 | CZ1 | BT3 | BT3 | H | H | H | H | H | H | Me |
| 1-1346 | CZ1 | BT6 | BT6 | H | H | H | H | H | H | Me |
| 1-1347 | CZ1 | BT7 | BT7 | H | H | H | H | H | H | Me |
| 1-1348 | CZ1 | CZ1 | CZ1 | H | H | H | H | H | H | Me |
| 1-1349 | P2 | P2 | P2 | H | H | H | H | H | H | Me |
| 1-1350 | P2 | P4 | P4 | H | H | H | H | H | H | Me |
| 1-1351 | P2 | P5 | P5 | H | H | H | H | H | H | Me |
| 1-1352 | P2 | P8 | P8 | H | H | H | H | H | H | Me |
| 1-1353 | P2 | BP1 | BP1 | H | H | H | H | H | H | Me |
| 1-1354 | P2 | BP2 | BP2 | H | H | H | H | H | H | Me |
| 1-1355 | P2 | BP3 | BP3 | H | H | H | H | H | H | Me |
| 1-1356 | P2 | BP4 | BP4 | H | H | H | H | H | H | Me |
| 1-1357 | P2 | BP7 | BP7 | H | H | H | H | H | H | Me |
| 1-1358 | P2 | TP1 | TP1 | H | H | H | H | H | H | Me |
| 1-1359 | P2 | TP4 | TP4 | H | H | H | H | H | H | Me |
| 1-1360 | P2 | TP12 | TP12 | H | H | H | H | H | H | Me |
| 1-1361 | P2 | TP15 | TP15 | H | H | H | H | H | H | Me |
| 1-1362 | P2 | TP16 | TP16 | H | H | H | H | H | H | Me |
| 1-1363 | P2 | TP17 | TP17 | H | H | H | H | H | H | Me |
| 1-1364 | P2 | TP18 | TP18 | H | H | H | H | H | H | Me |
| 1-1365 | P2 | QP38 | QP38 | H | H | H | H | H | H | Me |
| 1-1366 | P2 | QP44 | QP44 | H | H | H | H | H | H | Me |
| 1-1367 | P2 | QP64 | QP64 | H | H | H | H | H | H | Me |
| 1-1368 | P2 | NP1 | NP1 | H | H | H | H | H | H | Me |
| 1-1369 | P2 | NP3 | NP3 | H | H | H | H | H | H | Me |
| 1-1370 | P2 | NP4 | NP4 | H | H | H | H | H | H | Me |
| 1-1371 | P2 | NP5 | NP5 | H | H | H | H | H | H | Me |
| 1-1372 | P2 | NP6 | NP6 | H | H | H | H | H | H | Me |
| 1-1373 | P2 | NP7 | NP7 | H | H | H | H | H | H | Me |
| 1-1374 | P2 | NP8 | NP8 | H | H | H | H | H | H | Me |
| 1-1375 | P2 | NP11 | NP11 | H | H | H | H | H | H | Me |
| 1-1376 | P2 | NP13 | NP13 | H | H | H | H | H | H | Me |
| 1-1377 | P2 | NP14 | NP14 | H | H | H | H | H | H | Me |
| 1-1378 | P2 | NP15 | NP15 | H | H | H | H | H | H | Me |
| 1-1379 | P2 | NP16 | NP16 | H | H | H | H | H | H | Me |
| 1-1380 | P2 | NP17 | NP17 | H | H | H | H | H | H | Me |

Table 2-31

| | | | | | | | | | | |
|---|---|---|---|---|---|---|---|---|---|---|
| 1-1381 | P2 | NP18 | NP18 | H | H | H | H | H | H | Me |
| 1-1382 | P2 | PN1 | PN1 | H | H | H | H | H | H | Me |
| 1-1383 | P2 | FL1 | FL1 | H | H | H | H | H | H | Me |
| 1-1384 | P2 | TH3 | TH3 | H | H | H | H | H | H | Me |
| 1-1385 | P2 | BT1 | BT1 | H | H | H | H | H | H | Me |
| 1-1386 | P2 | BT3 | BT3 | H | H | H | H | H | H | Me |
| 1-1387 | P2 | BT6 | BT6 | H | H | H | H | H | H | Me |
| 1-1388 | P2 | BT7 | BT7 | H | H | H | H | H | H | Me |
| 1-1389 | P2 | CZ1 | CZ1 | H | H | H | H | H | H | Me |
| 1-1390 | TP1 | IN1 | IN1 | H | H | H | H | H | H | H |
| 1-1391 | TP1 | PY1 | PY1 | H | H | H | H | H | H | H |
| 1-1392 | TP1 | PY2 | PY2 | H | H | H | H | H | H | H |
| 1-1393 | TP1 | PY3 | PY3 | H | H | H | H | H | H | H |
| 1-1394 | TP1 | QN1 | QN1 | H | H | H | H | H | H | H |
| 1-1395 | TP1 | QN2 | QN2 | H | H | H | H | H | H | H |
| 1-1396 | TP1 | QN3 | QN3 | H | H | H | H | H | H | H |
| 1-1397 | NP11 | IN1 | IN1 | H | H | H | H | H | H | H |
| 1-1398 | NP11 | PY1 | PY1 | H | H | H | H | H | H | H |
| 1-1399 | NP11 | PY2 | PY2 | H | H | H | H | H | H | H |
| 1-1400 | NP11 | PY3 | PY3 | H | H | H | H | H | H | H |
| 1-1401 | NP11 | QN1 | QN1 | H | H | H | H | H | H | H |
| 1-1402 | NP11 | QN2 | QN2 | H | H | H | H | H | H | H |
| 1-1403 | NP11 | QN3 | QN3 | H | H | H | H | H | H | H |
| 1-1404 | TP1 | P1 | NP1 | H | H | H | H | H | H | H |
| 1-1405 | TP1 | P1 | NP11 | H | H | H | H | H | H | H |
| 1-1406 | TP1 | BP2 | NP1 | H | H | H | H | H | H | H |
| 1-1407 | TP1 | BP2 | NP11 | H | H | H | H | H | H | H |
| 1-1408 | NP11 | P1 | NP1 | H | H | H | H | H | H | H |
| 1-1409 | NP11 | P1 | NP11 | H | H | H | H | H | H | H |
| 1-1410 | NP11 | BP2 | NP1 | H | H | H | H | H | H | H |
| 1-1411 | NP11 | BP2 | NP11 | H | H | H | H | H | H | H |
| 1-1412 | TP1 | P1 | NP1 | H | H | H | H | H | H | Me |
| 1-1413 | TP1 | P1 | NP11 | H | H | H | H | H | H | Me |
| 1-1414 | TP1 | BP2 | NP1 | H | H | H | H | H | H | Me |
| 1-1415 | TP1 | BP2 | NP11 | H | H | H | H | H | H | Me |
| 1-1416 | NP11 | P1 | NP1 | H | H | H | H | H | H | Me |
| 1-1417 | NP11 | P1 | NP11 | H | H | H | H | H | H | Me |
| 1-1418 | NP11 | BP2 | NP1 | H | H | H | H | H | H | Me |
| 1-1419 | NP11 | BP2 | NP11 | H | H | H | H | H | H | Me |
| 1-1420 | NP11 | TP1 | TP1 | H | t-Bu | H | H | H | H | H |
| 1-1421 | NP11 | TP1 | TP1 | H | Me | H | H | H | H | H |
| 1-1422 | NP11 | TP1 | TP1 | H | TPM | H | H | H | H | H |
| 1-1423 | NP11 | TP1 | TP1 | H | PO | H | H | H | H | H |
| 1-1424 | NP11 | TP1 | TP1 | H | CY | H | H | H | H | H |
| 1-1425 | NP11 | TP1 | TP1 | H | Me | H | H | Me | H | H |
| 1-1426 | NP11 | TP1 | TP1 | H | t-Bu | H | H | t-Bu | H | H |

Among the specific examples described above, the preferred emission materials are compounds represented by (1-1), (1-15), (1-38), (1-102), (1-107), (1-113), (1-115), (1-153), (1-157), (1-158), (1-159), (1-163), (1-179), (1-185), (1-193), (1-206), (1-215), (1-216), (1-220), (1-221), (1-222), (1-225), (1-240), (1-246), (1-254), (1-259), (1-267), (1-268), (1-277), (1-295), (1-303), (1-310), (1-314), (1-315), (1-324), (1-331), (1-344), (1-351), (1-367), (1-372), (1-373), (1-376), (1-412), (1-413), (1-414), (1-418), (1-419), (1-422), (1-426), (1-435), (1-442), (1-459), (1-460), (1-464), (1-465), (1-468), (1-481), (1-488), (1-495), (1-505), (1-506), (1-510), (1-527), (1-534), (1-551), (1-552), (1-556), (1-573), (1-580), (1-597), (1-598), (1-601), (1-602), (1-603), (1-606), (1-619), (1-625), (1-626), (1-630), (1-636), (1-637), (1-642), (1-643), (1-644), (1-648), (1-649), (1-665), (1-672), (1-689), (1-690), (1-694), (1-695), (1-698), (1-711), (1-718), (1-735), (1-736), (1-740), (1-741), (1-757), (1-764), (1-781), (1-782), (1-786), (1-789), (1-799), (1-806), (1-981), (1-982), (1-989), (1-999), (1-1006), (1-1022), (1-1029), (1-1039), (1-1046), (1-1060), (1-1061), (1-1065), (1-1068), (1-1078), (1-1085), (1-1095), (1-1096), (1-1099), (1-1100), (1-1108), (1-1125), (1-1141), (1-1142), (1-1167), (1-1183), (1-1184), (1-1192), (1-1209), (1-1225), (1-1226), (1-1251), (1-1267), (1-1268), (1-1293), (1-1308), (1-1309), (1-1327), (1-1334), (1-1349), (1-1350), (1-1358), (1-1368), and (1-1375).

The more preferred emission materials are compounds represented by (1-15), (1-163), (1-179), (1-185), (1-193), (1-221), (1-277), (1-295), (1-303), (1-331), (1-372), (1-373), (1-376), (1-412), (1-413), (1-418), (1-419), (1-422), (1-426), (1-435), (1-442), (1-459), (1-464), (1-468), (1-488), (1-510), (1-534), (1-556), (1-580), (1-597), (1-601), (1-602), (1-603), (1-606), (1-625), (1-626), (1-630), (1-643), (1-648), (1-665), (1-698), (1-718), (1-735), (1-740), (1-741), (1-764), (1-1060), (1-1065), (1-1068), (1-1078), (1-1085), (1-1099), (1-1108), (1-1183), (1-1192), (1-1209), (1-1308), (1-1334), (1-1349), (1-1358) and (1-1375).

Further preferred emission materials are compounds represented by (1-163), (1-179), (1-331), (1-376), (1-412), (1-413), (1-418), (1-419), (1-422), (1-459), (1-464), (1-468), (1-556), (1-597), (1-606), (1-626), (1-648), (1-764), (1-1060), (1-1068), (1-1085), (1-1108), (1-1192), (1-1209), (1-1308), (1-1334), (1-1358) and (1-1375).

The emission material of the present invention can be synthesized by making use of known synthetic processes such as Suzuki coupling reaction. The Suzuki coupling reaction is a process in which aromatic halide is subjected to coupling with aromatic boric acid using a palladium catalyst in the presence of a base. A reaction route for obtaining the emission material (1) by the above process is shown in the following example:

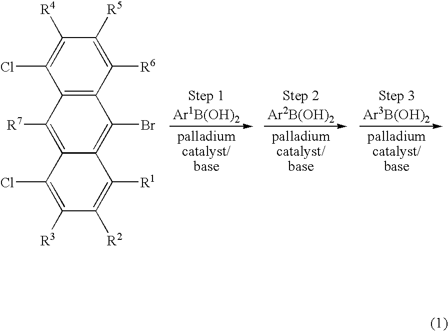

In the above formula, the codes of R¹ to R⁷ and Ar¹ to Ar³ are defined in the manners described above.

The examples of the palladium catalyst used in the above reaction are Pd(PPh₃)₄, PdCl₂(PPh₃)₂, Pd(OAc)₂, tris(dibenzylideneacetone)dipalladium (0) and tris(dibenzylideneacetone)dipalladium chloroform complex (0). A phosphine compound may be added, if necessary, to the above palladium compounds in order to accelerate the reaction. The examples of the phosphine compound are tri(tert-butyl)phosphine, tricyclohexyl phosphine, 1-(N,N-dimethylaminomethyl)-2-(di-tert-butylphosphino)ferrocene, 1-(N,N-dibutylaminomethyl)-2-(di-tert-butylphosphino)-ferrocene, 1-(methoxymethyl)-2-(di-tert-butylphosphino)ferrocene, 1,1′-bis(di-tert-butylphosphino)ferrocene, 2,2′-bis(di-tert-butylphosphino)-1,1′-binaphthyl, 2-methoxy-2′-(di-tert-butylphosphino)-1,1′-binaphthyl and 2-dicyclohexylphosphino-2′,6′-dimethoxybiphenyl. The examples of the base used in the above reaction are sodium carbonate, potassium carbonate, cesium carbonate, sodium hydrogencarbonate, sodium hydroxide, potassium hydroxide, barium hydroxide, sodium ethoxide, sodium tert-butoxide, sodium acetate, tripotassium phosphate and potassium fluoride. Further, the examples of the solvent used in the above reaction are benzene, toluene, xylene, N,N-dimethylformamide, tetrahydrofuran, diethyl ether, tert-butyl methyl ether, 1,4-dioxane, methanol, ethanol and isopropyl alcohol. The above solvents can suitably be selected according to the structures of the aromatic halide and the aromatic boric acid which are reacted. The solvents may be used alone or in the form of a mixed solvent.

The emission material of the present invention is a compound having strong fluorescent color in a solid state and can be used for emission of various colors, and it is particularly suited for emission of blue color. The emission material of the present invention has asymmetric molecular structure, and therefore it is liable to form an amorphous state in producing an organic EL device. The emission material of the present invention is excellent in heat resistance and stable as well in applying an electric field. Because of the reasons described above, the emission material of the present invention is excellent as an emission material for a field emission type device.

The emission material of the present invention has emission wavelength falling in wide range from short blue color extending to pure blue color, and therefore it is effective as a blue color host or a blue color dopant. Further, it can be used for a host emission material other than those of blue color. In particular, the emission material of the present invention is excellent as a blue color host. If the emission material of the present invention is used as a host material, energy transfer is efficiently carried out, and an emission device having high efficiency and long life is obtained.

The second present invention is an organic EL device in which an emission layer comprises the emission material of the present invention represented by Formula (1). The organic EL device of the present invention not only has high efficiency and long life but also has low drive voltage and high durability in storing and driving.

The organic EL device of the present invention has structures of various modes. Fundamentally, it comprises multilayer structure in which at least a hole transport layer, an emission layer and an electron transport layer are sandwiched between an anode and a cathode. The examples of the specific constitutions of the device are (1) anode/hole transport layer/emission layer/electron transport layer/cathode, (2) anode/hole injection layer/hole transport layer/emission layer/electron transport layer/cathode and (3) anode/hole injection layer/hole transport layer/emission layer/electron transport layer/electron injection layer/cathode.

The emission material of the present invention has high quantum efficiency, hole injection ability, hole transport ability, electron injection ability and electron transport ability, and therefore it can effectively be used as an emission material for an emission layer. In the organic EL device of the present invention, an emission layer can be formed from the emission material alone of the present invention. In the organic EL device of the present invention, combination of the emission material of the present invention with other emission materials makes it possible to improve emission luminance and emission efficiency and obtain emission of blue color, green color, red color and white color. In this case, the organic EL device of the present invention can contain the emission material of the present invention not only as a host but also as a dopant.

Other emission materials which can be used for the emission layer together with the emission material of the present invention are emission materials described in “Forefront in Full-scale Practical Use of Organic EL Display” (2002), p. 125 to 132, edited by Investigation and Research Section of Toray Research Center and published by Asahi High-Speed Print Co., Ltd. and emission materials described in p. 153 to 156 and triplet materials described in p. 170 to 172 of “Organic EL Materials and Displays” (2001), supervised by J. Kido and published by CMC Co., Ltd.

Compounds which can be used as the other emission materials are polycyclic aromatic compounds, hetero aromatic compounds, organic metal complexes, coloring matters, polymeric emission materials, styryl derivatives, coumarin derivatives, borane derivatives, oxazine derivatives, compounds having a spiro ring, oxadiazole derivatives and fluorene derivatives. The examples of the polycyclic aromatic compounds are anthracene derivatives, phenanthrene derivatives, naphthacene derivatives, pyrene derivatives, chrysene derivatives, perylene derivatives, coronene derivatives and rubrene derivatives. The examples of the heteroaromatic compounds are oxadiazole derivatives having a dialkylamino group or a diarylamino group, pyrazoloquinoline derivatives, pyridine derivatives, pyran derivatives, phenanthroline derivatives, silole derivatives, thiophene derivatives having a triphenylamine group and quinacridone derivatives. The examples of the organic metal complexes are complexes of zinc, aluminum, beryllium, europium, terbium, dysprosium, iridium and platinum with quinolinol derivatives, benzoxazole derivatives, benzothiazole derivatives, oxadiazole derivatives, thiadiazole derivatives, phenylpyridine derivatives, phenylbenzimidazole derivatives, pyrrole derivatives, pyridine derivatives and phenanthroline derivatives. The examples of the coloring matters include coloring matters such as xanthene derivatives, polymethine derivatives, porphyrin derivatives, coumarin derivatives, dicyanomethylenepyran derivatives, dicyanomethylenethiopyran derivatives, oxobenzanthracene derivatives, carbostyryl derivatives, perylene derivatives, benzoxazole derivatives, benzothiazole derivatives and benzimiazole derivatives. The examples of the polymeric emission materials are polyparaphenylvinylene derivatives, polythiophene derivatives, polyvinylcarbazole derivatives, polysilane derivatives, polyfluorene derivatives and polyparaphenylene derivatives. The examples of the styryl derivatives are amine-containing styryl derivatives and styrylarylene derivatives.

A dopant in using the emission material of the present invention as a blue color host is preferably perylene derivatives, amine-containing styryl derivatives, coumarin derivatives, borane derivatives, pyran derivatives, iridium complexes or platinum complexes. The examples of the perylene derivative are 3,10-bis(2,6-dimethylphenyl)perylene, 3,10-bis(2,4,6-trimethylphenyl)perylene, 3,10-diphenylperylene, 3,4-diphenylperylene, 2,5,8,11-tetra-tert-butylperylene, 3,4,9,10-tetraphenylperylene, 3-(1′-pyrenyl)-8,11-di(tert-butyl)perylene, 3-(9′-anthryl)-8,11-di(tert-butyl)perylene and 3,3′-bis(8,11-di(tert-butyl)perylenyl). The examples of the borane derivative are 1,8-diphenyl-10-(dimesitylboryl)anthracene, 9-phenyl-10-(dimethylboryl)anthracene, 4-(9′-anthryl)dimesitylborylnaphthalne, 4-(10′-phenyl-9′-anthryl)dimesitylborylnaphthalne, 9-(dimesitylboryl)anthracene, 9-(4′-biphenylyl)-10-(dimesitylboryl)anthracene and 9-(4′-(N-carbazolyl)phenyl)-10-(dimesitylboryl)anthracene. The examples of the coumarin derivative are coumarin-6 and coumarin-334.

The examples of the amine-containing styryl derivative are N,N,N′,N′-tetra(4-biphenylyl)-4,4′-diaminostilbene, N,N,N′,N′-tetra(1-naphthyl)-4,4′-diaminostilbene, N,N,N′,N′-tetra(2-naphthyl)-4,4′-diaminostilbene, N,N′-di(2-naphthyl)-N,N′-diphenyl-4,4′-diaminostilbene, N,N′-di(9-phenanthryl)-N,N′-diphenyl-4,4′-diaminostilbene, 4,4′-bis[4″-bis(diphenylamino)styryl]-biphenyl, 1,4-bis[4′-bis(diphenylamino)styryl]-benzene, 2,7-bis[4′-bis(diphenylamino)styryl]-9,9-dimethylfluorene, 4,4′-bis(9-ethyl-3-carbazovinylene)-biphenyl and 4,4′-bis(9-phenyl-3-carbazovinylene)-biphenyl.

The examples of the pyran derivative are DCM and DCJTB shown below:

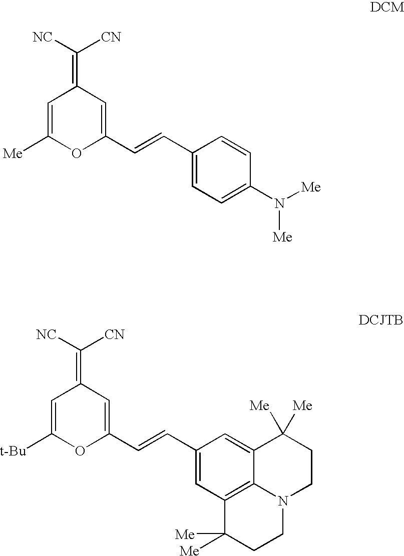

The examples of the iridium complex are Ir(ppy)₃ shown below and the like:

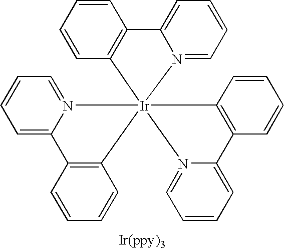

The examples of the platinum complex are PtOEP shown below and the like:

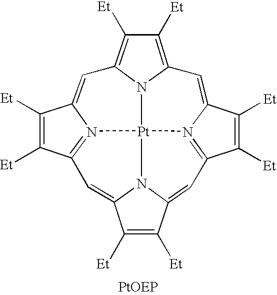

A host in using the emission material of the present invention as a blue color dopant is preferably anthracene derivatives, distyrylarylene derivatives, pyrene derivatives or fluorene derivatives. The examples of the anthracene derivative are 9-(2-naphthyl)-10-(3,5-diphenylphenyl)anthracene, 9-(1-naphthyl)-10-(3,5-diphenylphenyl)anthracene, 9-(2-naphthyl)-10-[3,5-di(2-naphthyl)phenyl]anthracene, 9-(2-naphthyl)-10-[3,5-di(1-naphthyl)phenyl]anthracene, 9-(1-naphthyl)-10-[3,5-di(2-naphthyl)phenyl]anthracene, 9-(1-naphthyl)-10-[3,5-di(1-naphthyl)phenyl]anthracene, 9,10-di(2-naphthyl)anthracene, 9,10-di(1-naphthyl)anthracene, 9,10-di(9-phenanthryl)anthracene, 9,10-bis(9,9-dimethyl-2-fluorenyl)anthracene, 2,3,6,7-tetramethyl-9,10-di(2-naphthyl)anthracene, 2,3,6,7-tetramethyl-9,10-di(1-naphthyl)anthracene, 2-tert-butyl-9,10-di(2-naphthyl)anthracene, 2-tert-butyl-9,10-di(1-naphthyl)anthracene, 9,10-bis[2-(2-naphthyl)phenyl]anthracene, 9,10-bis[2-(1-naphthyl)phenyl]anthracene, 9,10-bis[3,5-di(2-naphthyl)phenyl]anthracene, 9,10-bis[3,5-di(1-naphthyl)phenyl]anthracene, 9,10-bis(3,5-diphenylphenyl)anthracene, 9,10-bis[4-(3,5-diphenylphenyl)phenyl]anthracene, 9,10-bis[4-(2-naphthyl)phenyl]anthracene, 9,10-bis[4-(2,2-diphenylvinyl)phenyl]anthracene, 10,10′-bis(3,5-diphenylphenyl)-[9,9′]-bianthryl, 9,9′,10,10′-tetraphenyl-[2,2′]-bianthryl, 9,9′,10,10′-tetra(2-biphenylyl)-[2,2′]-bianthryl, 9,9′,10,10′-tetra(3-biphenylyl)-[2,2′]-bianthryl, 9,9′,10,10′-tetra(4-biphenylyl)-[2,2′]-bianthryl, 9,9′,10,10′-tetra(2-naphthyl)-[2,2′]-bianthryl and 9,9′,10,10′-tetra(1-naphthyl)-[2,2′]-bianthryl.

The examples of the distyrylarylene derivative are 4,4′-bis(2,2-diphenylvinyl)-biphenyl, 4,4′-bis[2,2-di(m-tolyl)vinyl]-biphenyl, 4,4′-bis(triphenylvinyl)-biphenyl, 4,4′-bis[2,2-bis-(4-tert-butylphenyl)vinyl]-biphenyl, 4,4′-bis[2-(4-tert-butylphenyl)-2-phenylvinyl]-biphenyl, 4,4′-bis[2,2-di(2-naphthyl)vinyl]-biphenyl, 4,4′-bis[2,2-di(1-naphthyl)vinyl]-biphenyl and 4,4′-bis(2,2-diphenylvinyl)-[1,1′]binaphthyl.

The examples of the pyrene derivative are 1-[3,5-di(2-naphthyl)phenyl]pyrene, 1,4-di(1-pyrenyl)benzene, 1,3,5-tri(1-pyrenyl)benzene, 1,4-di(1-pyrenyl)naphthalene and 2,6-di(1-pyrenyl)naphthalene.

The examples of the fluorene derivative are 1,3,5-tris(9,9-dimethyl-2-fluorenyl)benzene, 1,2,4,5-tetrakis(9,9-dimethyl-2-fluorenyl)benzene, 1,4-bis(9,9-dimethyl-2-fluorenyl)naphthalene and 2,6-bis(9,9-dimethyl-2-fluorenyl)naphthalene.

Those optionally selected from compounds which can be used as an electron transport compound in a photoconductive material and compounds which can be used for an electron injection layer and an electron transport layer in an organic EL device can be used as an electron transport material and an electron injection material which are used for the organic EL device of the present invention.

The examples of the above electron transport compound are quinolinol base metal complexes, pyridine derivatives, phenanthroline derivatives, diphenylquinone derivatives, perylene derivatives, oxadiazole derivatives, thiophene derivatives, triazole derivatives, thiadiazole derivatives, metal complexes of oxine derivatives, quinoxaline derivatives, polymers of quinoxaline derivatives, benzoxazole compounds, gallium complexes, pyrazole derivatives, perfluorinated phenylene derivatives, triazine derivatives, pyrazine derivatives, benzoquinoline derivatives, imidazopyridine derivatives and borane derivatives.

The preferred examples of the electron transport compound are quinolinol base metal complexes, pyridine derivatives or phenanthroline derivatives. The examples of the quinolinol base metal complexes are tris(8-hydroxyquinoline)aluminum (hereinafter abbreviated as ALQ), bis(10-hydroxybenzo[h]quinoline)beryllium, tris(4-methyl-8-hydroxyquinoline)aluminum and bis(2-methyl-8-hydroxyquinoline)-(4-phenylphenol)aluminum. The examples of the pyridine derivatives are 2,5-bis(6′-(2′,2″-bipyridyl)-1,1-dimethyl-3,4-diphenylsilol (hereinafter abbreviated as PyPySPyPy), 9,10-di(2′,2″-bipyridyl)anthracene, 2,5-di(2′,2″-bipyridyl)thiophene and 2,5-di(31,2″-bipyridyl)thiophene and 6′,6″-di(2-pyridyl)2,2′:4′:,3″:2″,2′″-quaterpyridine. The examples of the phenanthroline derivatives are 4,7-diphenyl-1,10-phenanthroline, 2,9-dimethyl-4,7-diphenyl-1,10-phenanthroline, 9,10-di(1,10-phenanthroline-2-yl)anthracene, 2,6-di(1,10-phenanthroline-5-yl)pyridine, 1,3,5-tri(1,10-phenanthroline-5-yl)benzene and 9,9′-bis(1,10-phenanthroline-5-yl). In particular, use of the phenanthroline derivatives for the electron transport layer or the electron injection layer makes it possible to realize the low voltage and the high efficiency.

Optional compounds selected from compounds which have so far conventionally been used as an electron transport material for a hole in a photoconductive material and publicly known compounds which are used for a hole injection layer and a hole transport layer in an organic EL device can be used as a hole injection material and a hole transport material which are used for the organic EL device of the present invention. The examples thereof are carbazole derivatives, triarylamine derivatives and phthalocyanine derivatives. The examples of the carbazole derivatives are N-phenylcarbazole and polyvinylcarbazole. The examples of the triarylamine derivatives are polymers having aromatic tertiary amine in a principal chain or a side chain, 1,1-bis(4-di-p-tolylaminophenyl)cyclohexane, N,N′-diphenyl-N,N′-di(3-methylphenyl)-4,4′-diaminobiphenyl, N,N′-diphenyl-N,N′-dinaphthyl-4,4′-diaminobiphenyl (hereinafter abbreviated as NPD), 4,4′,4″-tris{N-(3-methylphenyl)-N-phenylamino}triphenylamine and star burst amine derivatives. The examples of the phthalocyanine derivatives are non-metal phthalocyanine and copper phthalocyanine.

The respective layers constituting the organic EL device of the present invention can be formed by making thin films from materials to constitute the respective layers by a vapor deposition method, a spin cast method or a cast method. A film thickness of the respective layers thus formed shall not specifically be restricted and can suitably be set up according to the properties, and it falls in range of usually 2 nm to 5000 nm. The vapor deposition method is preferably adopted as a method for forming a thin film from the emission material in terms of the points that a homogeneous film is liable to be obtained and that pinholes are less liable to be formed. When the vapor deposition method is used to form a thin film, the vapor deposition conditions are varied depending on the kind of the emission material and a crystal structure and an aggregate structure which are targeted by a molecular cumulative film. The vapor deposition conditions are preferably set up in the ranges of usually boat heating temperature of 50 to 400° C., vacuum degree of 10⁻⁶ to 10⁻³ Pa, deposition speed of 0.01 to 50 nm/second, substrate temperature of −150 to +300° C. and film thickness of 5 nm to 5 μm.

The organic EL device of the present invention is preferably supported by a substrate in any of the structures described above. The substrate may be any one as long as it has mechanical strength, heat stability and transparency, and glass and transparent plastic film can be used. Metals, alloys, electroconductive compounds and mixtures thereof each having work function of larger than 4 eV can be used for the anode material. The examples thereof are metals such as Au and the like, CuI, indium tin oxide (hereinafter abbreviated as ITO), SnO₂ and ZnO.

Metals, alloys, electroconductive compounds and mixtures thereof each having work function of smaller than 4 eV can be used for the cathode material. The examples thereof are aluminum, calcium, magnesium, lithium, magnesium alloys and aluminum alloys. The examples of the alloys are aluminum/lithium fluoride, aluminum/lithium, magnesium/silver and magnesium/indium. At least one of the electrodes has preferably a light transmittance set to 10% or more in order to efficiently take out emission from the organic EL device. The electrodes are preferably controlled to sheet resistance of several hundred O/square or less. The film thickness is set, though depending on the properties of the electrode material, in range of usually 10 nm to 1 μm, preferably 10 to 400 nm. Such electrodes can be produced by forming thin films from the electrode substances described above by vapor deposition and sputtering.

Next, a method for preparing an organic EL device comprising anode/hole injection layer/hole transport layer/emission material of the present invention+dopant (emission layer)/electron transport layer/cathode each described above shall be explained as one example of methods for preparing an organic EL device using the emission material of the present invention. A thin film of an anode material is formed on a suitable substrate by a vapor deposition method to prepare an anode, and then the thin films of a hole injection layer and a hole transport layer are formed on the above anode. The emission material of the present invention and a dopant are codeposited thereon to form a thin film to thereby obtain an emission layer, and an electron transport layer is formed on the above emission layer. Further, a thin film comprising a material for a cathode is formed thereon by a vapor deposition method to prepare a cathode, whereby the intended organic EL device is obtained. In preparing the organic EL device described above, it can be prepared in the order of a cathode, an electron transport layer, an emission layer, a hole transport layer, a hole injection layer and an anode by upsetting the preparing order.

The emission material and the dopant are co-deposited by known method. That is, the substrate is mounted at an upper part of a vacuum bath, and two evaporation sources are mounted at a lower part thereof. The materials are evaporated from two evaporation sources at the same time, whereby both materials are deposited on the substrate while mixing. In this case, a partition board is disposed between two evaporation sources, and film thickness monitors are installed respectively in the vicinity of the substrate and the vicinity of the respective evaporation sources. A film having a desired mixed proportion can be obtained by evaporating the respective materials at a determined evaporation rate at the same time. Since the partition board is present between the evaporation sources, the film thickness monitors installed in the vicinity of the respective evaporation sources do not detect molecules evaporated from the other evaporation source, and therefore this is used to detect the respective evaporation rates. The film thickness monitor installed in the vicinity of the substrate detects molecules evaporated from both evaporation sources, and therefore this is used to always detect the piled film thickness, whereby the film having a desired film thickness can be formed on the substrate. Co-deposition in the present invention shall not be restricted to the method described above and can be carried out by known methods. The principle of co-deposition is disclosed as dual source deposition method in, for example, chapter 9.2 (p. 153) of Optical Technique Series II, Optical Thin Film (second edition), published on Oct. 10, 1986, Kyoritsu Shuppan Co., Ltd. The outline of practical apparatus is disclosed as an organic polymer deposition synthetic apparatus in, for example, third part, chapter 1, clause 1 (FIG. 8 at page 125) of Light-Thin Film Technical Manual (enlarged and revised edition), published on Aug. 31, 1992, The Optronics Co., Ltd. Further, a production method for an organic co-deposited film is disclosed in JP H14-76027 A/2002. Application to production of an organic EL device is disclosed in, for example, C. W. Tang, S. A. Van Slyke and C. H. Chen, J. Appl. Phys. 65 (9), 3610 to 3616, (1989).

When applying DC voltage to the organic +EL device thus obtained, it is applied with the polarity of the anode set to + and that of the cathode set to −, and when applying voltage of 2 to 40 V, emission can be observed from the transparent or translucent electrode sides (anode or cathode and both). Also, when applying AC voltage to the above organic EL device, emission is observed as well. The waveform of the alternating current applied may be optional.

The present invention shall be explained in further details with reference to examples.

### Example 1

### Synthesis of Compound (1-277)

10-Bromo-1,8-dichloroanthracene 3.26 g and 4-biphenylboronic acid 14.9 g were dissolved in 100 ml of N,N-dimethylformamide under nitrogen atmosphere, and Pd(OAc)₂ 0.34 g and 2-dicyclohexylphosphino-2′,6′-dimethoxybiphenyl 1.2 g were added thereto and stirred for one minute. Then, 19.1 g of tripotassium phosphate was added thereto and heated at 100° C. for 6 hours. After finishing heating, the reaction liquid was cooled down, and 200 ml of water was added thereto. A solid matter was filtered off and washed with water and methanol to obtain 6.2 g of a crude product. Then, it was extracted by Soxhlet method using 300 ml of toluene to obtain 4.5 g of the targeted product. The structure of the compound (1-277) was confirmed by MS spectrum and NMR measurement. Melting point: 351° C. (measuring equipment: Diamond DSC (manufactured by Perkin-Elmer Co., Ltd.); measuring conditions: cooling rate 200° C./min. and heating rate 10° C./min.)

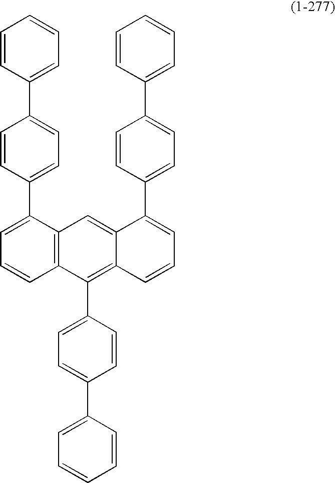

### Example 2

### Synthesis of Compound (1-373)

10-Bromo-1,8-dichloroanthracene 3.26 g and 2-biphenylboronic acid 14.9 g were dissolved in 100 ml of N,N-dimethylformamide under nitrogen atmosphere, and Pd(OAc)₂ 0.34 g and 2-dicyclohexylphosphino-2′,6′-dimethoxybiphenyl 1.2 g were added thereto and stirred for one minute. Then, 19.1 g of tripotassium phosphate was added thereto and heated at 100° C. for 12 hours. After finishing heating, the reaction liquid was cooled down, and 200 ml of water was added thereto. A solid matter was filtered off and washed with water and methanol to obtain 5.9 g of crude product. Then, it was subjected to column refining (solvent: heptane/toluene=3/1) with silica gel, and then 1.8 g of the targeted compound was obtained. The structure of the compound (1-373) was confirmed by an MS spectrum and NMR measurement. The other physical properties are shown below.

Glass transition temperature: 91° C.; melting point: 229° C. (measuring equipment: Diamond DSC (manufactured by Perkin-Elmer Co., Ltd.); measuring conditions: cooling rate 200° C./min. and heating rate 10° C./min.)

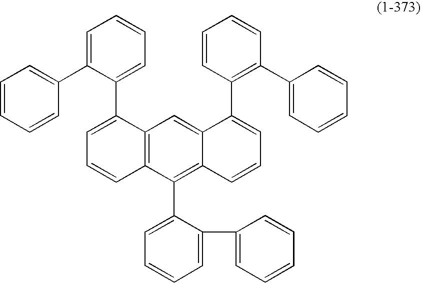

### Example 3

### Synthesis of Compound (1-412)

10-Bromo-1,8-dichloroanthracene 3.26 g and m-terphenyl-5′-boronic acid 2.74 g were dissolved in 100 ml of mixed solvent of toluene and ethanol (toluene/ethanol=4/1) under nitrogen atmosphere, and 0.58 g of tetrakis(triphenylphosphine)palladium (0) was added thereto and stirred for 5 minutes. Then, 10 ml of 2M sodium carbonate aqueous solution was added thereto, and the solution was refluxed for 8 hours. After finishing heating, the reaction liquid was cooled to separate an organic layer, and it was washed with saturated brine and then dried on anhydrous magnesium sulfate. A solid matter obtained by removing the drying agent and distilling the solvent off under reduced pressure was subjected to column refining (solvent: heptane/toluene=3/1) with silica gel, and then 4.6 g of an intermediate compound 1,8-dichloro-10-(m-terphenyl-5′-yl)anthracene was obtained.

Tris(dibenzylideneacetone)dipalladium (0) 0.266 g and tri-tert-butylphosphine 0.117 g were dissolved in 50 ml of 1,4-dioxane, and 4.6 g of 1,8-dichloro-10-(m-terphenyl-5′-yl)anthracene described above, 3.54 g of phenylboronic acid and 3.7 g of potassium fluoride each were added thereto, followed by heating the mixture at 90° C. for 90 hours. After finishing heating, the reaction liquid was cooled down and subjected to short column with silica gel (solvent:toluene). Thereafter, it was subjected to column refining (solvent:heptane/toluene=2/1) with silica gel, and then 3.6 g of the targeted compound was obtained. The structure of the compound (1-412) was confirmed by MS spectrum and NMR measurement. The other physical properties are shown below. Glass transition temperature (Tg): 108° C.; melting point: 257° C. (measuring equipment: Diamond DSC (manufactured by Perkin-Elmer Co., Ltd.); measuring conditions: cooling rate 200° C./min. and heating rate 10° C./min.)

### Example 4

### Synthesis of Compound (1-422)

10-Bromo-1,8-dichloroanthracene 3.26 g and m-terphenyl-5′-boronic acid 20.56 g were dissolved in 100 ml of N,N-dimethylformamide under nitrogen atmosphere, and Pd(OAc)₂ 0.34 g and 2-dicyclohexylphosphino-2′,6′-dimethoxybiphenyl 1.2 g were added thereto and stirred for one minute. Then, 19.1 g of tripotassium phosphate was added thereto and heated at 100° C. for 8 hours. After finishing heating, the reaction liquid was cooled down, and 200 ml of water was added thereto. A solid matter was filtered off and washed with water and methanol to obtain 8.5 g of a crude product. Thereafter, it was subjected to column refining (solvent: heptane/toluene=2/1) with silica gel, and then 6.2 g of the targeted compound was obtained. The structure of the compound (1-422) was confirmed by MS spectrum and NMR measurement. The other physical properties are shown below.

Glass transition temperature: 145° C.; melting point: 307° C. (measuring equipment: Diamond DSC (manufactured by Perkin-Elmer Co., Ltd.); measuring conditions: cooling rate 200° C./min. and heating rate 10° C./min.)

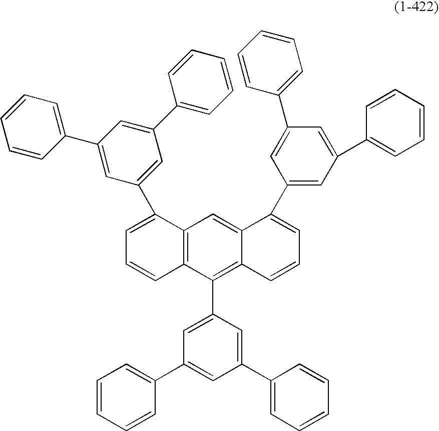

### Example 5

### Synthesis of Compound (1-626)

10-Bromo-1,8-dichloroanthracene 3.26 g and 2-naphthaleneboronic acid 12.9 g were dissolved in 100 ml of N,N-dimethylformamide under nitrogen atmosphere, and Pd(OAc)₂ 0.34 g and 2-dicyclohexylphosphino-2′,6′-dimethoxybiphenyl 1.2 g were added thereto and stirred for one minute. Then, 19.1 g of tripotassium phosphate was added thereto and heated at 100° C. for 4 hours. After finishing heating, the reaction liquid was cooled down, and 200 ml of water was added thereto. A solid matter was filtered off and washed with water and methanol to obtain 5.5 g of a crude product. Thereafter, it was subjected to column refining (solvent: heptane/toluene=2/1) with silica gel, and then 4.2 g of the targeted compound was obtained. The structure of the compound (1-626) was confirmed by an MS spectrum and NMR measurement. The other physical properties are shown below.

Glass transition temperature: 109° C.; melting point: 277° C. (measuring equipment: Diamond DSC (manufactured by Perkin-Elmer Co., Ltd.); measuring conditions: cooling rate 200° C./min. and heating rate 10° C./min.)

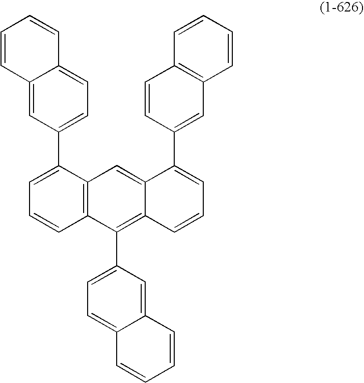

Suited selection of the compounds which are the raw materials makes it possible to synthesize the other emission materials of the present invention by a method corresponding to the synthetic example described above.

### Example 6

A glass substrate (manufactured by Tokyo Sanyo Vacuum Co., Ltd.) of 26 mm×28 mm×0.7 mm on which ITO was deposited in a thickness of 150 nm was used for a transparent supporting substrate. This transparent supporting substrate was fixed on a substrate holder of a commercial vacuum deposition apparatus (manufactured by ULVAC KIKO Inc.), and installed therein were a molybdenum-made boat source for deposition containing copper phthalocyanine, a molybdenum-made boat source for deposition containing NPD, a molybdenum-made boat source for deposition containing the compound (1-412), a molybdenum-made boat source for deposition containing ALQ, a molybdenum-made boat source for deposition containing lithium fluoride and a tungsten-made boat source for deposition containing aluminum. A pressure of the vacuum chamber was reduced down to 1×10⁻³ Pa, and the boat source for deposition containing copper phthalocyanine was heated to deposit copper phthalocyanine so that a film thickness of 20 nm was obtained to thereby form a hole injection layer. Then, the boat source for deposition containing NDP was heated to deposit NDP so that a film thickness of 30 nm was obtained to thereby form a hole transport layer. Next, the molybdenum-made boat source for deposition containing the compound (1-412) was heated to deposit the compound (1-412) so that a film thickness of 35 nm was obtained to thereby form an emission layer. Then, the boat source for deposition containing ALQ was heated to deposit ALQ so that a film thickness of 15 nm was obtained to thereby form an electron transport layer. The above deposit rates were 0.1 to 0.2 nm/second. Thereafter, the boat source for deposition containing lithium fluoride was heated to deposit lithium fluoride at a deposit rate of 0.003 to 0.01 nm/second so that a film thickness of 0.5 nm was obtained, and then the boat source for deposition containing aluminum was heated to deposit aluminum at a deposit rate of 0.2 to 0.5 nm/second so that a film thickness of 100 nm was obtained, whereby an organic EL device was obtained. A DC voltage of about 4.8 V was applied with the ITO electrode set to an anode and the lithium fluoride/aluminum electrode set to a cathode, and a current of about 4 mA/cm² passed to obtain emission of blue color having emission efficiency of 2.5 μm/W and wavelength of 434 nm. Further, the half lifetime of the device was 200 hours at an initial luminance of 1000 cd/m² when it was driven at a constant current of 50 mA/cm².

### Example 7

An organic EL device was obtained by a method corresponding to Example 6, except that ALQ used for the electron transport layer in Example 6 was changed to PyPySPyPy. A DC voltage of about 3 V was applied with the ITO electrode set to an anode and the lithium fluoride/aluminum electrode set to a cathode, and a current of about 3 mA/cm² passed to obtain emission of blue color having emission efficiency of 3.6 μm/W and wavelength of 436 nm. Further, the half lifetime of the device was 160 hours at an initial luminance of 1500 cd/m² when it was driven at a constant current of 50 mA/cm².

### Example 8

A glass substrate (manufactured by Tokyo Sanyo Vacuum Co., Ltd.) of 26 mm×28 mm×0.7 mm on which ITO was deposited in a thickness of 150 nm was used for a transparent supporting substrate. This transparent supporting substrate was fixed on a substrate holder of a commercial vacuum deposition apparatus (manufactured by ULVAC KIKO, Inc.), and installed therein were a molybdenum-made boat source for deposition containing copper phthalocyanine, a molybdenum-made boat source for deposition containing NPD, a molybdenum-made boat source for deposition containing the compound (1-412), a molybdenum-made boat source for deposition containing 3,10-bis(2,6-dimethylphenyl)perylene, a molybdenum-made boat source for deposition containing ALQ, a molybdenum-made boat source for deposition containing lithium fluoride and a tungsten-made boat source for deposition containing aluminum. A pressure of the vacuum chamber was reduced down to 1×10⁻³ Pa, and the boat source for deposition containing copper phthalocyanine was heated to deposit copper phthalocyanine so that a film thickness of 20 nm was obtained to thereby form a hole injection layer. Then, the boat source for deposition containing NDP was heated to deposit NDP so that a film thickness of 30 nm was obtained to thereby form a hole transport layer. Next, the molybdenum-made boat source for deposition containing the compound (1-412) and the molybdenum-made boat source for deposition containing 3,10-bis(2,6-dimethylphenyl)perylene were heated to codeposit both compounds so that a film thickness of 35 nm was obtained to thereby form an emission layer. In this case, a doping concentration of 3,10-bis(2,6-dimethylphenyl)perylene was about 1% by weight. Then, the boat source for deposition containing ALQ was heated to deposit ALQ so that a film thickness of 15 nm was obtained to thereby form an electron transport layer. The above deposit rates were 0.1 to 0.2 nm/second. Thereafter, the boat source for deposition containing lithium fluoride was heated to deposit lithium fluoride at a deposit rate of 0.003 to 0.01 nm/second so that a film thickness of 0.5 nm was obtained, and then the boat source for deposition containing aluminum was heated to deposit aluminum at a deposit rate of 0.2 to 0.5 nm/second so that a film thickness of 100 nm was obtained, whereby an organic EL device was obtained. A DC voltage of about 4.5 V was applied with the ITO electrode set to an anode and the lithium fluoride/aluminum electrode set to a cathode, and a current of about 1.9 mA/cm² passed to obtain emission of blue color having emission efficiency of 4 μm/W and wavelength of 469 nm. Further, the half lifetime of the device was 350 hours at an initial luminance of 1850 cd/m² when it was driven at a constant current of 50 mA/cm².

### Example 9

An organic EL device was obtained by a method corresponding to Example 8, except that 3,10-bis(2,6-dimethylphenyl)perylene used for the dopant in Example 8 was changed to N,N,N′,N′-tetra(4-biphenylyl)-4,4′-diaminostilbene. A DC voltage of about 4.5 V was applied with the ITO electrode set to an anode and the lithium fluoride/aluminum electrode set to a cathode, and a current of about 1.3 mA/cm² passed to obtain emission of blue color having emission efficiency of 5.3 μm/W and wavelength of 480 nm. Further, the half lifetime of the device was 300 hours at an initial luminance of 3100 cd/m² when it was driven at a constant current of 50 mA/cm².

### Example 10

An organic EL device was obtained by a method corresponding to Example 9, except that the compound (1-412) used in Example 9 was changed to the compound (1-422). A DC voltage of about 4.7 V was applied with the ITO electrode set to an anode and the lithium fluoride/aluminum electrode set to a cathode, and a current of about 1.7 mA/cm² passed to obtain emission of blue color having emission efficiency of 5.0 μm/W and wavelength of 479 nm. Further, the half lifetime of the device was 280 hours at an initial luminance of 3000 cd/m² when it was driven at a constant current of 50 mA/cm².

### Example 11

An organic EL device was obtained by a method corresponding to Example 8, except that ALQ used for the electron transport layer in Example 8 was changed to PyPySPyPy. A DC voltage of about 3 V was applied with the ITO electrode set to an anode and the lithium fluoride/aluminum electrode set to a cathode, and a current of about 1 mA/cm² passed to obtain emission of blue color having emission efficiency of 6 μm/W and wavelength of 468 nm. Further, the half lifetime of the device was 250 hours at an initial luminance of 2600 cd/m² when it was driven at a constant current of 50 mA/cm².

### Example 12

A glass substrate (manufactured by Tokyo Sanyo Vacuum Co., Ltd.) of 26 mm×28 mm×0.7 mm on which ITO was deposited in a thickness of 150 nm was used for a transparent supporting substrate. This transparent supporting substrate was fixed on a substrate holder of a commercial vacuum deposition apparatus (manufactured by ULVAC KIKO, Inc.), and installed therein were a molybdenum-made boat source for deposition containing copper phthalocyanine, a molybdenum-made boat source for deposition containing NPD, a molybdenum-made boat source for deposition containing 9-(2-naphthyl)-10-(3,5-diphenylphenyl)anthracene, a molybdenum-made boat source for deposition containing the compound (1-412), a molybdenum-made boat source for deposition containing ALQ, a molybdenum-made boat source for deposition containing lithium fluoride and a tungsten-made boat source for deposition containing aluminum. A pressure of the vacuum chamber was reduced down to 1×10⁻³ Pa, and the boat source for deposition containing copper phthalocyanine was heated to deposit copper phthalocyanine so that a film thickness of 20 nm was obtained to thereby form a hole injection layer. Then, the boat source for deposition containing NDP was heated to deposit NDP so that a film thickness of 30 nm was obtained to thereby form a hole transport layer. Next, the molybdenum-made boat source for deposition containing 9-(2-naphthyl)-10-(3,5-diphenylphenyl)anthracene and the molybdenum-made boat source for deposition containing the compound (1-412) were heated to codeposit both compounds so that a film thickness of 35 nm was obtained to thereby form an emission layer. In this case, a doping concentration of the compound (1-412) was about 1% by weight. Then, the boat source for deposition containing ALQ was heated to deposit ALQ so that a film thickness of 15 nm was obtained to thereby form an electron transport layer. The above deposit rates were 0.1 to 0.2 nm/second. Thereafter, the boat source for deposition containing lithium fluoride was heated to deposit lithium fluoride at a deposit rate of 0.003 to 0.01 nm/second so that a film thickness of 0.5 nm was obtained, and then the boat source for deposition containing aluminum was heated to deposit aluminum at a deposit rate of 0.2 to 0.5 nm/second so that a film thickness of 100 nm was obtained, whereby an organic EL device was obtained. A DC voltage of about 4.7 V was applied with the ITO electrode set to an anode and the lithium fluoride/aluminum electrode set to a cathode, and a current of about 3.9 mA/cm² passed to obtain emission of blue color having emission efficiency of 3 μm/W and wavelength of 435 nm. Further, the half lifetime of the device was 210 hours at an initial luminance of 1300 cd/m² when it was driven at a constant current of 50 mA/cm².

### INDUSTRIAL APPLICABILITY

The emission material of the present invention is excellent in emission of blue color. Use of this emission material makes it possible to obtain an organic EL device having high emission efficiency, low drive voltage, excellent heat resistance and long life. A display unit having high performance such as display of full color can be prepared by using the organic EL device of the present invention.

1. An emission material represented by the following Formula (1): 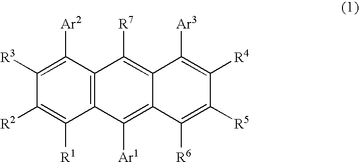
wherein R¹ to R⁷ are independently hydrogen, alkyl having 1 to 24 carbon atoms or cycloalkyl having 3 to 24 carbon atoms; optional —CH₂— in the above alkyl having 1 to 24 carbon atoms may be replaced by —O—, and optional —CH₂— other than —CH₂— directly bonded to the anthracene ring may be replaced by arylene having 6 to 24 carbon atoms;
optional hydrogens in the above cycloalkyl having 3 to 24 carbon atoms may be replaced by alkyl having 1 to 24 carbon atoms or aryl having 6 to 50 carbon atoms;
Ar¹ is one selected from the group consisting of non-condensed aryl having 6 to 50 carbon atoms, 2-naphthyl, 9-phenanthryl, 6-chrysenyl, 2-triphenylenyl, 2-fluorenyl, 9-carbazolyl, 2-thienyl and 2-benzothienyl;
optional hydrogens in the above groups may be replaced by alkyl having 1 to 24 carbon atoms, cycloalkyl having 3 to 12 carbon atoms, aryl having 6 to 24 carbon atoms or heteroaryl; optional —CH₂— in the above alkyl having 1 to 24 carbon atoms may be replaced by —O—, and optional —CH₂— other than —CH₂— directly bonded to the above groups may be replaced by arylene having 6 to 24 carbon atoms; optional hydrogens in the above cycloalkyl having 3 to 24 carbon atoms may be replaced by alkyl having 1 to 24 carbon atoms or aryl having 6 to 24 carbon atoms; optional hydrogens in the above aryl having 6 to 24 carbon atoms may be replaced by alkyl having 1 to 12 carbon atoms, cycloalkyl having 3 to 12 carbon atoms or aryl having 6 to 24 carbon atoms, and optional hydrogens in the above heteroaryl may be replaced by alkyl having 1 to 12 carbon atoms, cycloalkyl having 3 to 12 carbon atoms or aryl having 6 to 24 carbon atoms; and
Ar² and Ar³ are independently non-condensed aryl having 6 to 50 carbon atoms, condensed aryl having 10 to 50 carbon atoms or heteroaryl.

2. The emission material as described in claim 1, wherein R¹ to R⁷ are independently hydrogen, methyl or tert-butyl, and Ar¹ is non-condensed aryl having 6 to 50 carbon atoms.

3. The emission material as described in claim 1, wherein R¹ to R⁷ are independently hydrogen, methyl or tert-butyl, and Ar¹ is phenyl, biphenylyl, terphenylyl or quaterphenylyl.

4. The emission material as described in claim 1, wherein R¹ to R⁷ are independently hydrogen, methyl or tert-butyl, and Ar¹ is 2-naphthyl, 9-phenanthryl, 6-chrysenyl, 2-triphenylenyl, 2-fluorenyl, 9-carbazolyl, 2-thienyl or 2-benzothienyl.

5. An emission material represented by the following Formula (1): 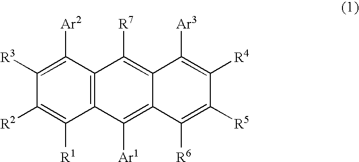
wherein R¹ to R⁷ are independently hydrogen, methyl or tert-butyl, and Ar¹ is non-condensed aryl represented by Formula (2);
Ar² and Ar³ are independently phenyl, 4-tert-butylphenyl, 4-(9-carbazolyl)phenyl, 2-biphenylyl, 3-biphenylyl, 4-biphenylyl, m-terphenyl-5′-yl, 3,5-di(2-naphthyl)phenyl, p-quaterphenyl-3′-yl, m-quaterphenyl-3-yl, o-quaterphenyl-2-yl, 1-naphthyl, 4-phenyl-1-naphthyl, 4-(9-carbazolyl)-1-naphthyl, 2-naphthyl, 6-(m-terphenyl-5′-yl)-2-naphthyl, 6-(2-naphthyl)-2-naphthyl, 9-phenanthryl, 2-benzothienyl or 3-phenyl-2-benzothienyl; 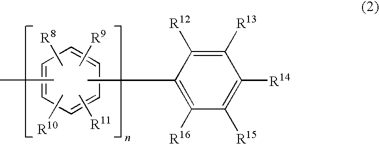
wherein n is an integer of 0 to 8;
R⁸ to R¹⁶ are independently hydrogen, alkyl having 1 to 24 carbon atoms, cycloalkyl having 3 to 24 carbon atoms, aryl having 6 to 24 carbon atoms or heteroaryl; optional —CH₂— in the above alkyl having 1 to 24 carbon atoms may be replaced by —O—, and optional —CH₂— other than —CH₂— directly bonded to the benzene ring may be replaced by arylene having 6 to 24 carbon atoms; optional hydrogens in the above cycloalkyl having 3 to 24 carbon atoms may be replaced by alkyl having 1 to 24 carbon atoms or aryl having 6 to 24 carbon atoms; optional hydrogens in the above aryl having 6 to 24 carbon atoms may be replaced by alkyl having 1 to 24 carbon atoms, cycloalkyl having 3 to 24 carbon atoms or aryl having 6 to 24 carbon atoms; and optional hydrogens in the above heteroaryl may be replaced by alkyl having 1 to 12 carbon atoms, cycloalkyl having 3 to 12 carbon atoms or aryl having 6 to 24 carbon atoms.

6. The emission material as described in claim 5, wherein Ar¹ is phenyl, biphenylyl, terphenylyl or quaterphenylyl in which optional hydrogens may be replaced by methyl, tert-butyl, phenyl, 2-naphthyl, 1-naphthyl, 2-benzothienyl, 3-phenyl-2-benzothienyl or 9-carbazolyl.

7. The emission material as described in claim 5, wherein Ar¹ is phenyl, 2-biphenylyl, 3-biphenylyl, 4-biphenylyl, m-terphenyl-5′-yl, m-quaterphenyl-3-yl or o-quaterphenyl-3-yl in which optional hydrogens may be replaced by methyl, tert-butyl, phenyl, 2-naphthyl, 1-naphthyl, 2-benzothienyl, 3-phenyl-2-benzothienyl or 9-carbazolyl.

8. An emission material represented by the following Formula (1): 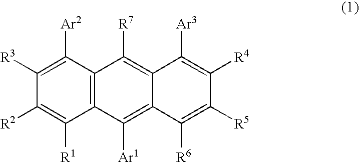
wherein R¹ to R⁷ are independently hydrogen, methyl or tert-butyl;
Ar¹ is 2-naphthyl, 9-phenanthryl, 6-chrysenyl, 2-triphenylenyl, 2-fluorenyl, 9-carbazolyl, 2-thienyl or 2-benzothienyl in which optional hydrogens may be replaced by methyl, tert-butyl, phenyl, m-terphenyl-5′-yl, 2-naphthyl, 1-naphthyl, 2-benzothienyl, 3-phenyl-2-benzothienyl or 9-carbazolyl; and
Ar² and Ar³ are independently phenyl, 4-tert-butylphenyl, 4-(9-carbazolyl)phenyl, 2-biphenylyl, 3-biphenylyl, 4-biphenylyl, m-terphenyl-5′-yl, 3,5-di(2-naphthyl)phenyl, p-quaterphenyl-3′-yl, m-quaterphenyl-3-yl, o-quaterphenyl-2-yl, 1-naphthyl, 4-phenyl-1-naphthyl, 4-(9-carbazolyl)-1-naphthyl, 2-naphthyl, 6-(m-terphenyl-5′-yl)-2-naphthyl, 6-(2-naphthyl)-2-naphthyl, 9-phenanthryl, 2-benzothienyl or 3-phenyl-2-benzothienyl.

9. The emission material as described in claim 5, wherein Ar¹ is one selected from phenyl, 4-tert-butylphenyl and 4-(9-carbazolyl)phenyl.

10. The emission material as described in claim 5, wherein Ar¹ is one selected from 2-biphenylyl, 3-biphenylyl and 4-biphenylyl.

11. The emission material as described in claim 5, wherein Ar¹ is m-terphenyl-5′-yl.

12. The emission material as described in claim 5, wherein Ar¹ is 3,5-di(2-naphthyl)phenyl.

13. The emission material as described in claim 5, wherein Ar¹ is m-quaterphenyl-3-yl or o-quaterphenyl-3-yl.

14. The emission material as described in claim 8, wherein Ar¹ is one selected from 2-naphthyl, 6-(m-terphenyl-5′-yl)-2-naphthyl, 6-(2-naphthyl)-2-naphthyl and 6-(9-carbazolyl)-2-naphthyl.

15. The emission material as described in claim 8, wherein Ar¹ is 9-phenanthryl.

16. The emission material as described in claim 8, wherein Ar¹ is 9-carbazolyl.

17. The emission material as described in claim 8, wherein Ar¹ is 2-benzothienyl or 3-phenyl-2-benzothienyl.

18. The emission material as described in any of claims 9 to 17, wherein R¹ to R⁶ are hydrogens; R⁷ is hydrogen or methyl; and Ar² and Ar³ are one selected from phenyl, 4-tert-butylphenyl and 4-(9-carbazolyl)phenyl.

19. The emission material as described in any of claims 9 to 17, wherein R¹ to R⁶ are hydrogens; R⁷ is hydrogen or methyl; and Ar² and Ar³ are one selected from 2-biphenylyl, 3-biphenylyl and 4-biphenylyl.

20. The emission material as described in any of claims 9 to 17, wherein R¹ to R⁶ are hydrogens; R⁷ is hydrogen or methyl; and Ar² and Ar³ are m-terphenyl-5′-yl.

21. The emission material as described in any of claims 9 to 17, wherein R¹ to R⁶ are hydrogens; R⁷ is hydrogen or methyl; and Ar² and Ar³ are 3,5-di(2-naphthyl)phenyl.

22. The emission material as described in any of claims 9 to 17, wherein R¹ to R⁶ are hydrogens; R⁷ is hydrogen or methyl; and Ar² and Ar³ are one selected from p-quaterphenyl-3′-yl, m-quaterphenyl-3-yl and o-quaterphenyl-2-yl.

23. The emission material as described in any of claims 9 to 17, wherein R¹ to R⁶ are hydrogens; R⁷ is hydrogen or methyl; and Ar² and Ar³ are one selected from 1-naphthyl, 4-phenyl-1-naphthyl and 4-(9-carbazolyl)-1-naphthyl.

24. The emission material as described in any of claims 9 to 17, wherein R¹ to R⁶ are hydrogens; R⁷ is hydrogen or methyl; and Ar² and Ar³ are one selected from 2-naphthyl, 6-(m-terphenyl-5′-yl)-2-naphthyl and 6-(2-naphthyl)-2-naphthyl.

25. The emission material as described in any of claims 9 to 17, wherein R¹ to R⁶ are hydrogens; R⁷ is hydrogen or methyl; and Ar² and Ar³ are 9-phenanthryl.

26. The emission material as described in any of claims 9 to 17, wherein R¹ to R⁶ are hydrogens; R⁷ is hydrogen or methyl; and Ar² and Ar³ are 2-benzothienyl or 3-phenyl-2-benzothienyl.

27. An organic electroluminescent device comprising a substrate and provided thereon at least a hole transport layer, an emission layer and an electron transport layer which are sandwiched between an anode and a cathode, wherein the above emission layer comprises the emission material as described in any of claims 1 to 17.
